(19) United States
(12) Patent Application Publication
(10) Pub. No.: US 2005/0005319 A1
(43) Pub. Date: Jan. 6, 2005

(54) Method of determining the function of nucleotide sequences and the proteins they encode by transfecting the same into a host

(75) Inventors: Guy della-Cioppa, Vacaville, CA (US); Robert L. Erwin, Davis, CA (US); Wayne P. Fitzmaurice, Vacaville, CA (US); Kathleen M. Hanley, Vacaville, CA (US); Moaro H. Kumagai, Kailua, HI (US); John A. Lindbo, Vacaville, CA (US); David R. McGee, Vacaville, CA (US); Hal S. Padgett, Vacaville, CA (US); Gregory P. Pogue, Vacaville, CA (US)

Correspondence Address: LARGE SCALE BIOLOGY CORPORATION, 3333 VACA VALLEY PARKWAY, SUITE 1000, VACAVILLE, CA, 95688, US

(73) Assignee: LARGE SCALE BIOLOGY CORPORATION, Vacaville, CA (US)

(21) Appl. No.: 10/893,056

(22) Filed: Jul. 16, 2004

Related U.S. Application Data
(63) Continuation of application No. 10/072,438, filed on Feb. 5, 2002.
Application No. 10/072,438 is a continuation of application No. 09/232,170, filed on Jan. 15, 1999, now abandoned.
Application No. 09/232,170 is a continuation-in-part of application No. 09/008,186, filed on Jan. 16, 1998, now abandoned.

(51) Int. Cl. A01H 1/00; C12N 15/82; C12N 15/87

(52) U.S. Cl. 800/260; 800/278

(57) ABSTRACT

The present invention provides methods for rapidly determining the function of nucleic acid sequences by transfecting the same into a host organism to effect expression. Phenotypic and biochemical changes produced thereby are then analyzed to ascertain the function of the nucleic acids which have been transfected into the host organism. The invention also provides methods for silencing endogenous genes by transfecting hosts with nucleic acid sequences to effect expression of the same. The present invention also provides methods for selecting desired functions of RNAs and proteins by the use of virus vectors to express libraries of nucleic acid sequence variants. Moreover, the present invention provides methods for inhibiting an endogenous protease of a plant host.

This application is a Continuation application of U.S. application Ser. No. 10/072,438, filed Feb. 5, 2002; which is a Continuation application of U.S. application Ser. No. 09/232,170, filed Jan. 15, 1999; which is a Continuation-In-Part of U.S. application Ser. No. 09/008,186, filed Jan. 16, 1998.

### FIELD OF THE INVENTION

The present invention relates generally to the field of molecular biology and plant genetics. Specifically, the present invention relates to a method for determining the function of nucleotide sequences and genes by transfecting the same into a host.

### BACKGROUND OF THE INVENTION

Great interest exists in launching genome projects in plants comparable to the human genome project. Valuable and basic agricultural plants, including by way of example but without limitation, corn, soybeans and rice are targets for such projects because the information obtained thereby may prove very beneficial for increasing world food production and improving the quality and value of agricultural products. The United States Congress is considering launching a corn genome project. By helping to unravel the genetics hidden in the corn genome, the project could aid in understanding and combating common diseases of grain crops. It could also provide a big boost for efforts to engineer plants to improve grain yields and resist drought, pests, salt, and other extreme environmental conditions. Such advances are critical for a world population expected to double by 2050. Currently, there are four species which provide 60% of all human food: wheat, rice, corn, and potatoes, and the strategies for increasing the productivity of these plants is dependent on rapid discovery of the function of unknown gene sequences determined as a result of genomics research. Moreover, such information could identify genes and products encoded by genes useful for human and animal healthcare such as pharmaceuticals.

One strategy that has been proposed to assist in such efforts is to create a database of expressed sequence tags (ESTs) that can be used to identify expressed genes. Accumulation and analysis of expressed sequence tags (ESTs) have become an important component of genome research. EST data may be used to identify gene products and thereby accelerate gene cloning. Various sequence databases have been established in an effort to store and relate the tremendous amount of sequence information being generated by the ongoing sequencing efforts. Some have suggested sequencing 500,000 ESTs for corn and 100,000 ESTs each for rice, wheat, oats, barley, and sorghum. Efforts at sequencing the genomes of plant species will undoubtedly rely upon these computer databases to share the sequence data as it is generated. Arabidopsis thaliana may be an attractive target for gene function discovery because a very large set of ESTs have already been produced in this organism, and these sequences tag more than 50% of the expected Arabidopsis genes.

Estimates of several of the important grain genome sizes (in reference to microbes and humans) have been suggested. These include Oryza sativa (rice) at about 430 million bases or about 20,000 genes, Sorghum bicolor (sorghum) at about 760 million bases or about 30,000 genes, Zea mays (corn) at about 2 billion bases or about 30,000 genes, and Triticum aestivum (wheat) at about 16 billion bases or about 30,000 genes.

Potential use of the sequence information so generated is enormous if gene function can be determined. It may become possible to engineer commercial seeds for agricultural use to convey any number of desirable traits to food and fiber crops and thereby increase agricultural production and the world food supply. Research and development of commercial seeds has so far focused primarily on traditional plant breeding, however there has been increased interest in biotechnology as it relates to plant characteristics. Knowledge of the genomes involved and the function of genes contained therein for both monocotyledonous and dicotyledonous plants is essential to realizing positive effects from such technology.

The impact of genomic research in seeds is potentially far reaching. For example, gene profiling in cotton can lead to an understanding of the types of genes being expressed primarily in fiber cells. The genes or promoters derived from these genes may be important in genetic engineering of cotton fiber for increased strength or for “built-in” fiber color. In plant breeding, gene profiling coupled to physiological trait analysis can lead to the identification of predictive markers that will be increasingly important in marker assisted breeding programs. Mining the DNA sequence of a particular crop for genes important for yield, quality, health, appearance, color, taste, etc., are applications of obvious importance for crop improvement.

Work has been conducted in the area of developing suitable vectors for expressing foreign DNA and RNA in plant hosts. Ahlquist, U.S. Pat. Nos. 4,885,248 and 5,173,410 describes preliminary work done in devising transfer vectors which might be useful in transferring foreign genetic material into a plant host for the purpose of expression therein. Additional aspects of hybrid RNA viruses and RNA transformation vectors are described by Ahlquist et al. in U.S. Pat. Nos. 5,466,788, 5,602,242, 5,627,060 and 5,500,360, all of which are incorporated herein by reference. Donson et al., U.S. Pat. Nos. 5,316,931, 5,589,367 and 5,866,785, incorporated herein by reference, demonstrate for the first time plant viral vectors suitable for the systemic expression of foreign genetic material in plants. Donson et al. describe plant viral vectors having heterologous subgenomic promoters for the systemic expression of foreign genes. Carrington et al., U.S. Pat. No. 5,491,076, describe particular potyvirus vectors also useful for expressing foreign genes in plants. The expression vectors described by Carrington et al. are characterized by utilizing the unique ability of viral polyprotein proteases to cleave heterologous proteins from viral polyproteins. These include Potyviruses such as Tobacco Etch Virus. Additional suitable vectors are described in U.S. Pat. No. 5,811,653 and U.S. patent application Ser. No. 08/324,003, both of which are incorporated herein by reference.

Construction of plant RNA viruses for the introduction and expression of non-viral foreign genes in plants has also been demonstrated by Brisson et al., Methods in Enzymology 118:659 (1986), Guzman et al., Communications in Molecular Biology: Viral Vectors, Cold Spring Harbor Laboratory, pp. 172-189 (1988), Dawson et al., Virology 172:285-292 (1989), Takamatsu et al., EMBO J. 6:307-311 (1987), French et al., Science 231:1294-1297 (1986), and Takamatsu et al., FEBS Letters 269:73-76 (1990). However, these viral vectors have not been shown capable of systemic spread in the plant and expression of the non-viral foreign genes in the majority of plant cells in the whole plant. Moreover, many of these viral vectors have not proven stable for the maintenance of non-viral foreign genes. However, the viral vectors described by Donson et al., in U.S. Pat. Nos. 5,316,931, 5,589,367, and 5,866,785, Turpen in U.S. Pat. No. 5,811,653, Carrington et al. in U.S. Pat. No. 5,491,076, and in co-pending U.S. patent application Ser. No. 08/324,003, have proven capable of infecting plant cells with foreign genetic material and systemically spreading in the plant and expressing the non-viral foreign genes contained therein in plant cells locally or systemically. Likely, additional vehicles having greater infectivity and enhanced local or systemic expression of foreign genetic material will be developed either independently or as improvements of the vectors described in the patents and pending applications noted above. All patents, patent applications, and references cited in the instant application are hereby incorporated by reference.

The recombinant plant viral nucleic acids and recombinant viruses such as those demonstrated by Donson et al. which have been demonstrated to infect plant cells and express the foreign genetic material systemically are generally characterized as comprising a native plant viral subgenomic promoter, at least one non-native plant viral subgenomic promoter, a plant viral coat protein coding sequence, and at least one non-native nucleic acid sequence. The value of using such plant viral nucleic acids to effect systemic expression of non-native nucleic acids in a plant host is significant. This tool, if coupled with a rational design for elucidating the function of the non-native nucleic acids, would make significant strides in understanding the large amount of sequence information produced by sequencing efforts.

### SUMMARY OF THE INVENTION

In one aspect, the present invention is directed to a method of determining the function of nucleic acid sequences including genes and the proteins they encode in host organisms such as bacteria, yeast, plants, or animals, by transfecting the nucleic acid sequences into the organisms in a manner so as to effect localized or systemic expression of the nucleic acid sequences. The present inventors have determined methods for determining the function of nucleic acid sequences and the proteins they encode by transfecting organisms with nucleic acids of interest thereby providing a more rapid means for elucidating the function of these nucleic acids including genes and subsequently utilizing the rapidly expanding information in the field of genomics.

In one embodiment, a nucleic acid is introduced into a plant host wherein the plant host may be a monocotyledonous or dicotyledonous plant, plant tissue or plant cell. Preferably, the nucleic acid may be introduced by way of a plant viral nucleic acid. Such plant viral nucleic acids are stable for the maintenance and transcription or expression of non-native nucleic acid sequences and are capable of locally or systemically transcribing or expressing such sequences in the plant host. Especially preferred recombinant plant viral nucleic acids useful in the methods of the present invention comprise a native plant viral subgenomic promoter, a plant viral coat protein coding sequence, and at least one non-native nucleic acid sequence.

Some viral vectors used in accordance with the present invention may be encapsidated by the coat proteins encoded by the recombinant plant virus. The recombinant plant viral nucleic acid or recombinant plant virus is used to infect appropriate hosts such as plants. The recombinant plant viral nucleic acid is capable of replication in the host, localized or systemic spread in the host, and transcription or expression of the non-native nucleic acid in the host to produce the desired product. Such products may be for example, useful polypeptides or proteins including enzymes, complex biomolecules, ribozymes, or polypeptides or protein products resulting from positive-sense or anti-sense RNA expression. Moreover, in alternate embodiments, the nucleic acid of interest may be expressed with the genomic DNA or RNA of the viral vectors and hence be under the control of a genomic promoter.

Some other viral vectors used in accordance with the present invention comprise recombinant animal viruses or portions thereof. Likewise, such animal viral vectors are useful to infect appropriate hosts such as animals. The recombinant animal viral nucleic acid is capable if replication in the host, systemic or localized spread in the host, and transcription or expression of the non-native nucleic acid in the host to produce the desired product.

In another embodiment, the present method uses a viral expression vector encoding for at least one protein non-native to the vector that is released from at least one polyprotein expressed by said vector by proteolytic processing.

In yet other preferred embodiments according to the present method, recombinant plant viruses are used which encode for the expression of a fusion between a plant viral coat protein and the amino acid product of the nucleic acid of interest.

In yet other preferred embodiments according to the present method, a nucleic acid sequence of interest including a gene may be placed within any suitable vector construct such as a virus for infecting the host organism. That is, the present method may be practiced without concern for the position of the nucleic acid sequence of interest within the vector used to infect the host organism. The invention is not intended to be limited to any particular viral constructs but specifically contemplates using all operable constructs. Those skilled in the art will understand that these embodiments are representative only of many constructs which may be useful to produce localized or systemic expression of nucleic acids in host organisms such as plants. All such constructs are contemplated and intended to be within the scope of the present invention.

Those of skill in the art will readily understand that there are many methods to determine the function of the nucleic acid once localized or systemic expression in a host, such as a plant, plant cell, transgenic plant, animal or animal cell is attained. In one embodiment the function of a nucleic acid may be determined by complementation analysis. That is, the function of the nucleic acid of interest may be determined by observing the endogenous gene or genes whose function is replaced or augmented by introducing the nucleic acid of interest. A discussion of such phenomenon is provided by Napoli et al., The Plant Cell 2:279-289 (1990). In a second embodiment, the function of a nucleic acid may be determined by analyzing the biochemical alterations in the accumulation of substrates or products from enzymatic reactions according to any one of the means known by those skilled in the art. In a third embodiment, the function of a nucleic acid may be determined by observing phenotypic changes in the host by methods including morphological, macroscopic or microscopic analysis. In a fourth embodiment, the function of a nucleic acid may be determined by observing any changes in biochemical pathways which may be modified in the host organism as a result of expression of the nucleic acid. In a fifth embodiment, the function of a nucleic acid may be determined utilizing techniques known by those skilled in the art to observe inhibition of endogenous gene expression in the cytoplasm of cells as a result of expression of the nucleic acid. In a sixth embodiment, the function of a nucleic acid may be determined utilizing techniques known by those skilled in the art to observe changes in the RNA or protein profile as a result of expression of the nucleic acid. In a seventh embodiment, the function of a nucleic acid may be determined by selection of organisms such as plants or human cells and tissues capable of growing or maintaining viability in the presence of noxious or toxic substances, such as, for example herbicides and pharmaceutical ingredients.

A second aspect of the present invention is a method of silencing endogenous genes in a host by introducing nucleic acids into the host by way of a viral nucleic acid such as a plant or animal viral nucleic acid suitable to produce expression of a nucleic acid in a transfected host. In one embodiment, the host is a plant, but those skilled in the art will understand that other hosts such as bacteria, yeast and animals including humans may also be utilized. This method utilizes the principle of post-transcription gene silencing of the endogenous host gene homolog. Since the replication mechanism of the transfected non-native nucleic acid produces both sense and antisense RNA sequences, the orientation of the non-native nucleic acid insert is not crucial to providing gene silencing. Particularly, this aspect of the invention is especially useful for silencing a multigene family as is frequently found in plants. The prior art has not demonstrated an effective means for silencing a multigene family in plants.

A third aspect of the present invention is a method for selecting desired functions of RNAs and proteins by the use of virus vectors to express libraries of nucleic acid sequence variants. Libraries of sequence variants may be generated by means of in vitro mutagenenisis and/or recombination. Rapid in vitro evolution can be used to improve virus-specific or protein-specific functions. In particular, plant RNA virus expression vectors may be used as tools to bear libraries containing variants of nucleic acid, genes from virus, plant or other sources, and to be applied to plants or plant cells such that the desired altered effects in the RNA or protein products can be determined, selected and improved. In a preferred embodiment, nucleic acid shuffling techniques may be employed to construct shuffled gene libraries. Random, semi-random or known sequences of virus origin may also be inserted in virus expression vectors between native virus sequences and foreign gene sequences, to increase the genetic stability of foreign genes in expression vectors as well as the translation of the foreign gene and the stability of the mRNA encoding the foreign gene in vivo. The desired function of RNA and protein may include the promoter activities, replication properties, translational efficiencies, movement properties (local and systemic), signaling pathway, or virus host range, among others. The desired function alteration can be identified by assaying infected plants and the nature of mutation can be determined by analysis of sequence variants in the virus vector.

Methods to increase the representation of gene sequences in virus expression libraries may also be achieved by bypassing the genetic bottleneck of propagation in E. coli. For example, in one of the preferred embodiments of the instant invention, cell-free methods may be used to clone sequence libraries or individual arrayed sequences into virus expression vectors and reconstruct an infectious virus, such that the final ligation product can be transcribed and the resulting RNA can be used for plant or plant cell inoculation/infection with the output being gene function discovery or protein production.

Techniques to screen sequence libraries can be introduced into RNA viruses or RNA virus vectors as populations or individuals in parallel to identify individuals with novel and augmented virus-encoded functions in replication and virus movement, foreign gene sequence retention in vectors and proper folding, activity and expression of protein products, novel gene expression, effects on host metabolism, and resistance or susceptibility of plants to exogenous agents.

Variation in the sequence of a native virus gene(s) or heterologous nucleotide sequence(s) may be introduced into an RNA virus or an RNA virus expression vector by many methods as a means to screen a population of variants in batch or individuals in parallel for novel properties exhibited by the virus itself or conferred on the host plant or cell by the virus vector. Variant populations can be transfected as populations or individual clones into “host”: 1) protoplasts; 2) whole plants; or 3) inoculated leaves of whole plants and screened for various traits including protein expression (increase or decrease), RNA expression (increase or decrease), secondary metabolites or other host property gained or loss as a result of the virus infection.

For treatment of hosts with agents that result in cell death or down regulation in general metabolic function, a virus vector, which simultaneously expressed the green fluorescent protein (GFP) or other selectable marker gene and the variant sequence, is used to screen quantitatively for levels of resistance or sensitivity to the agent in question conferred upon the host by the variant sequence expressed from the viral vector. By quantitatively screening pools or individual infection events, those viruses containing unique variant sequences allowing sustained metabolic life of host are identified by fluorescence under long wave UV light. Those that do not confer this phenotype will fail to or poorly fluoresce. In this manner, high throughput screening in multi-well dishes in plate readers is possible where the average fluorescence of the well would be expressed as a ratio of the adsorption (measuring the cell mass) thereby giving a comparable quantitative value. This technique enables screening of populations or individuals followed by rescue of the sequence from virus vectors conferring desired trait by RT-PCR and re-screening of particular variant sequences in secondary screens.

The functions of transcription factors or factors contributing to the signal transduction pathway of host cells are monitored by using specific proteomic, mRNA or metanomic traits to be assayed following transfection with a virus expression library. The contribution of a particular protein or product to a valuable trait may be known from the literature, but a new mode of enhanced or reduced expression could be identified by finding the factors that respond to cellular signals that in turn alter its particular expression. For example, transcription factors regulating the expression of defense proteins such as systemin peptides, or protease inhibitors could be identified by transfecting hosts with virus libraries and the expression of systemin or protease inhibitors or their RNAs be directly assayed. Conversely, the promoters responsible for expressing these genes could be genetically fused to the green fluorescent protein and introduced into hosts as transient expression constructs or into stable transformed host cells/tissues. The resulting cells would be transfected with viral vector libraries. Hosts now could be screened rapidly by following relative GFP expression following vector transfection. Likewise, coupling the transfecting of hosts with virus libraries with the treatment of plants with methyl jasmonate could identify sequences that reverse or enhance the gene induction events induced by this metabolite. This approach could be applied to other factors involved in promotion of higher biomass in plants such as Leafy or DET2. The expression of these factors could be directly assayed or via promoters genetically fused to GFP. This technique will enable screening of populations or individuals followed by rescue of the sequence from virus vectors conferring desired trait by RT-PCR and re-screening of particular variant sequences in secondary screens.

A fourth aspect of the present invention is a method for inhibiting an endogenous protease of a plant host comprising the step of treating the plant host with a compound which induces the production of an endogenous inhibitor of said protease. In a preferred embodiment, jasmonic acid may be used to treat the plant host to induce the production of an endogenous inhibitor of an endogenous protease. In another preferred embodiment, the treatment of the plant host with a compound results an increased representation of an exogenous nucleic acid or the protein product thereof. In particular, transgenic hosts expressing protease inhibitors may be used to decrease the degradation ofproteins expressed by virus expression vectors. In a preferred embodiment, jasmonic acid may be used to treat plants infected with virus expression vectors to decrease degradation of proteins expressed by virus expression vectors.

A fifth aspect of the present invention are genes and fragments thereof, nucleotide sequences, and gene products obtained by way of the method of the present invention. The present invention features expressing selected nucleotide sequences in a host organism. Those of skill in the art will readily appreciate that the gene products of such nucleotide sequences may be isolated using techniques known to those skilled in the art. Such gene products may exhibit biological activity as pharmaceuticals, herbicides, and other similar functions.

### BRIEF DESCRIPTION OF THE FIGURES

FIG. 1 depicts the vector TT01/PSY+.

FIG. 2 represents the vector TTO1A/PDS+.

FIG. 3 represents the vector TT01A/Ca CCS+.

FIG. 4 represents the vector TTU51 CTP CrtB.

FIG. 5 represents the vector TTOSA1 CTP CrtI 491.

FIG. 6 represents the Erwinia herbicola phytoene desaturase gene (plasmid pAU211).

FIG. 7 represents the plasmid KS+/CrtI* 491.

FIG. 8 represents the plasmid pBS736.

FIG. 9 represents the plasmid pBS 712.

FIG. 10 represents the 72 kDa gene product of the genomic clone encoding alcohol oxidase ZZA1.

FIG. 11 represents the plasmid TTOS1APE ZZA1.

FIG. 12 represents the plasmid TTO1A 103L.

FIG. 13 represents the plasmid TTU51A QSEO #3.

FIG. 14 represents the plasmid KS+TVCVK #23.

FIG. 15 represents the plasmid pBS735.

FIG. 16 represents the plasmid pBS740.

FIG. 17 represents the plasmid pBS723.

FIG. 18 represents the plasmid pBS731.

FIG. 19 represents the plasmid pBS740 AT #120.

FIG. 20 represents the nucleotide sequence alignment of 740 AT #120 to human ADP-ribosylation factor (ARF3) M33384.

FIG. 21 represents the plasmid pBS740 AT #88.

FIG. 22 represents the nucleotide sequence alignment of 740 AT #88 to L33574 mRNA for rhodopsin.

FIG. 23 represents the nucleotide sequence alignment of 740 AT #88 to X07797 Octopus mRNA for rhodopsin.

FIG. 24 represents the protein sequence alignment of 740 AT #88 to an Arabidopsis est ORF ATTS2938.

FIG. 25 represents the protein sequence alignment of 740 AT #88 to Octopus rhodopsin P31356.

FIG. 26 represents amino acid sequence comparison of 740 AT #2441 to tobacco RAN-B1 GTP binding protein.

FIG. 27 represents nucleotide sequence comparison of 740 AT #2441 to human RAN GTP-binding protein.

FIG. 28 represents a schematic diagram of cell free cloning.

### DETAILED DESCRIPTION OF THE INVENTION

In one aspect, the present invention is directed to a method of determining the function of a nucleic acid sequence including a gene and a protein encoded thereby in an organism such as bacteria, fungi, yeast, animals and plants by transfecting the nucleic acid sequence into the organism. The present inventors have determined methods for determining the function of nucleic acid sequences by transfecting organisms with the nucleic acids thereby providing a more rapid means for determining gene function and utilizing the rapidly expanding sequence information in the field of genomics.

In one embodiment, a nucleic acid is introduced into a plant host. Preferably, the nucleic acid may be introduced by way of a viral nucleic acid. Such recombinant viral nucleic acids are stable for the maintenance and transcription or expression of non-native nucleic acid sequences and are capable of systemically transcribing or expressing such non-native sequences in the plant host. Especially preferred recombinant plant viral nucleic acids usefull in the present invention comprise a native plant viral subgenomic promoter, a plant viral coat protein coding sequence, and at least one non-native nucleic acid sequence.

In a second embodiment, plant viral nucleic acid sequences used in the method of the present invention are characterized by the deletion of the native coat protein coding sequence and comprise a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid. The recombinant plant viral nucleic acid may contain one or more additional native or non-native subgenomic promoters. Each non-native subgenomic promoter is capable of transcribing or expressing adjacent genes or nucleic acid sequences in the plant host and incapable of recombination with each other and with native subgenomic promoters. One or more non-native nucleic acids may be inserted adjacent to the native plant viral subgenomic promoter or the native and non-native plant viral subgenomic promoters if more than one nucleic acid sequence is included. Moreover, it is specifically contemplated that two or more heterologous non-native subgenomic promoters may be used. The non-native nucleic acid sequences may be transcribed or expressed in the host plant under the control of the subgenomic promoter to produce the products of the nucleic acids of interest.

In a third embodiment, plant viral nucleic acids are used in the present invention wherein the native coat protein coding sequence is placed adjacent one of the non-native coat protein subgenomic promoters instead of a non-native coat protein coding sequence.

In a fourth embodiment, plant viral nucleic acids are used in the present invention wherein the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce the product of the non-native nucleic acid. Alternatively, the native coat protein coding sequence may be replaced by a non-native coat protein coding sequence.

The viral vectors used in accordance with the present invention may be encapsidated by the coat proteins encoded by the recombinant plant virus. The recombinant plant viral nucleic acid or recombinant plant virus is used to infect appropriate hosts such as plants. The recombinant plant viral nucleic acid is capable of replication in the host, localized or systemic spread in the host, and transcription or expression of the non-native nucleic acid in the host to produce the desired product. Such products may be for example, therapeutics and other useful polypeptides or proteins including enzymes, complex biomolecules, ribozymes, or polypeptides or protein products resulting from positive-sense or anti-sense RNA expression. Moreover, the nucleic acid of interest may be under the control of a genomic promoter and therefore be expressed with the genome of the virus.

In another embodiment, the present method uses a viral expression vector encoding at least one protein non-native to the vector that is released from at least one polyprotein expressed by said vector by proteolytic processing catalyzed by at least one protease in said polyprotein wherein said vector comprises at least one promoter, DNA having a sequence which codes for at least one polyprotein from a polyprotein-producing virus, at least one restriction site flanking a 3′ terminus of said DNA and a cloning vehicle. Additional embodiments use a viral expression vector encoding for at least one protein non-native to the vector that is released from at least one polyprotein expressed by the vector by proteolytic processing catalyzed by at least one protease in the polyprotein wherein the vector comprises at least one promoter, DNA having a sequence which codes for at least one polyprotein from a polyprotein-producing virus, may contain at least one restriction site flanking a 3′ terminus of said cDNA and a cloning vehicle. Preferred embodiments include using a potyvirus as the polyprotein-producing virus, and especially preferred embodiments may use TEV (tobacco etch virus). A more detailed description of such vectors useful according to the method of the present invention may be found in U.S. Pat. No. 5,491,076 which is incorporated herein by reference.

In yet other preferred embodiments according to the present method, recombinant plant viruses are used which encode for the expression of a fusion between a plant viral coat protein and the amino acid product of the nucleic acid of interest. Such a recombinant plant virus provides for high level expression of a nucleic acid of interest. The location or locations where the viral coat protein is joined to the amino acid product of the nucleic acid of interest may be referred to as the fusion joint. A given product of such a construct may have one or more fusion joints. The fusion joint may be located at the carboxyl terminus of the viral coat protein or the fusion joint may be located at the amino terminus of the coat protein portion of the construct. In instances where the nucleic acid of interest is located internal with respect to the 5′ and 3′ residues of the nucleic acid sequence encoding for the viral coat protein, there are two fusion joints. That is, the nucleic acid of interest may be located 5′, 3′, upstream, downstream or within the coat protein. In some embodiments of such recombinant plant viruses, a “leaky” start or stop codon may occur at a fusion joint which sometimes does not result in translational termination. A more detailed description of some recombinant plant viruses according to this embodiment of the invention may be found in co-pending U.S. patent application Ser. No. 08/324,003 the disclosure of which is incorporated herein by reference.

In yet other embodiments according to the present method, a nucleic acid sequence of interest or a gene may be placed within any suitable vector construct such as a virus for infecting the host organism. That is, the present method may be practiced without concern for the position of the nucleic acid sequence of interest within the vector used to infect the host organism. The invention is not intended to be limited to any particular viral constructs but specifically contemplates using all operable constructs. Specifically, those skilled in the art may choose to transfer DNA or RNA of any size up to and including an entire genome into a host organism in order to determine the function thereof.

Those skilled in the art will understand that these embodiments are representative only of many constructs which may be useful to produce localized or systemic expression of nucleic acids in host organisms such as plants. All such constructs are contemplated and intended to be within the scope of the present invention.

In order to provide an even clearer and more consistent understanding of the specification and the claims, including the scope given herein to such terms, the following definitions are provided:

Adjacent: A position in a nucleotide sequence proximate to and 5′ or 3′ to a defined sequence. Generally, adjacent means within 2 or 3 nucleotides of the site of reference.

Animal cell: A single functional cell found within an animal organism. Animal tissue refers to one or more cells grouped or organized to perform one or more functions. Animal organ refers to one or more tissues morphologically arranged to perform one or more functions within an organism.

Anti-Sense Inhibition: A type of gene regulation based on cytoplasmic, nuclear or organelle inhibition of gene expression due to the presence in a cell of an RNA molecule complementary to at least a portion of the mRNA being translated. It is specifically contemplated that DNA molecules may be from either an RNA virus or mRNA from the host cells genome or from a DNA virus.

Cell Culture: A proliferating group of cells which may be in either an undifferentiated or differentiated state, growing contiguously or non-contiguously.

Chimeric Sequence or Gene: A nucleotide sequence derived from at least two heterologous parts. The sequence may comprise DNA or RNA.

Coding Sequence: A deoxyribonucleotide or ribonucleotide sequence which, when either transcribed and translated or simply translated, results in the formation of a cellular polypeptide or a ribonucleotide sequence which, when translated, results in the formation of a cellular polypeptide.

Compatible: The capability of operating with other components of a system. A vector or plant or animal viral nucleic acid which is compatible with a host is one which is capable of replicating in that host. A coat protein which is compatible with a viral nucleotide sequence is one capable of encapsidating that viral sequence.

Complementation Analysis: As used herein, this term refers to observing the changes produced in an organism when a nucleic acid sequence is introduced into that organism after a selected gene has been deleted or mutated so that it no longer functions fully in its normal role. A complementary gene to the deleted or mutated gene can restore the genetic phenotype of the selected gene.

Constitutive expression: Gene expression which features substantially constant or regularly cyclical gene transcription. Generally, genes which are constitutively expressed are substantially free of induction from an external stimulus.

Differentiated cell: A cell which has substantially matured to perform one or more biochemical or physiological finctions.

Dual Heterologous Subgenomic Promoter Expression System (DHSPES): a plus stranded RNA vector having a dual heterologous subgenomic promoter expression system to increase, decrease, or change the expression of proteins, peptides or RNAs, preferably those described in U.S. Pat. Nos. 5,316,931, 5,811,653, 5,589,367, and 5,866,785, the disclosure of which is incorporated herein by reference.

Expressed sequence tags (ESTs): Relatively short single-pass DNA sequences obtained from one or more ends of cDNA clones and RNA derived therefrom. They may be present in either the 5′ or the 3′ orientation. ESTs have been shown useful for identifying particular genes.

Expression: The term as used herein is meant to incorporate one or more of transcription, reverse transcription and translation.

Gene: A discrete nucleic acid sequence responsible for producing one or more cellular products and/or performing one or more intercellular or intracellular functions.

Gene silencing: A reduction in gene expression. A viral vector expressing gene sequences from a host may induce gene silencing of homologous gene sequences.

Growth cycle: As used herein, the term is meant to include the replication of a nucleus, an organelle, a cell, or an organism.

Host: A cell, tissue or organism capable of replicating a nucleic acid such as a vector or plant viral nucleic acid and which is capable of being infected by a virus containing the viral vector or viral nucleic acid. This term is intended to include prokaryotic and eukaryotic cells, organs, tissues or organisms, where appropriate. Bacteria, fungi, yeast, animal (cell, tissues, or organisms), and plant (cell, tissues, or organisms) are examples of a host.

Induction: The terms “induce”, “induction” and “inducible” refer generally to a gene and a promoter operably linked thereto which is in some manner dependent upon an external stimulus, such as a molecule, in order to actively transcribe and/or translate the gene.

Infection: The ability of a virus to transfer its nucleic acid to a host or introduce a viral nucleic acid into a host, wherein the viral nucleic acid is replicated, viral proteins are synthesized, and new viral particles assembled. In this context, the terms “transmissible” and “infective” are used interchangeably herein. The term is also meant to include the ability of a selected nucleic acid sequence to integrate into a genome, chromosome or gene of a target organism.

Multigene family: A set of genes descended by duplication and variation from some ancestral gene. Such genes may be clustered together on the same chromosome or dispersed on different chromosomes. Examples of multigene families include those which encode the histones, hemoglobins, immunoglobulins, histocompatibility antigens, actins, tubulins, keratins, collagens, heat shock proteins, salivary glue proteins, chorion proteins, cuticle proteins, yolk proteins, and phaseolins.

Non-Native: Any RNA or DNA sequence that does not normally occur in the cell or organism in which it is placed. Examples include recombinant plant viral nucleic acids and genes or ESTs contained therein. That is, an RNA or DNA sequence may be non-native with respect to a viral nucleic acid. Such an RNA or DNA sequence would not naturally occur in the viral nucleic acid. Also, an RNA or DNA sequence may be non-native with respect to a host organism. That is, such a RNA or DNA sequence would not naturally occur in the host organism. Conversely, the term non-native does not imply that an RNA or DNA sequence must be non-native with respect to both a viral nucleic acid and a host organism concurrently. The present invention specifically contemplates placing an RNA or DNA sequence which is native to a host organism into a viral nucleic acid in which it is non-native.

Nucleic acid: As used herein the term is meant to include any DNA or RNA sequence from the size of one or more nucleotides up to and including a complete gene sequence. The term is intended to encompass all nucleic acids whether naturally occurring in a particular cell or organism or non-naturally occurring in a particular cell or organism.

Nucleic acid of interest: The term is used interchangeably with the term “nucleic acid” and is intended to refer to the nucleic acid sequence whose function is to be determined. The sequence will normally be non-native to the viral vector but may be native or non-native to the host organism.

Organism: The term organism and “host organism” as used herein is specifically intended to include animals including humans, plants, viruses, fungi, and bacteria.

Phenotypic Trait: An observable, measurable or detectable property resulting from the expression or suppression of a gene or genes.

Plant Cell: The structural and physiological unit of plants, consisting of a protoplast and the cell wall.

Plant Organ: A distinct and visibly differentiated part of a plant, such as root, stem, leaf or embryo.

Plant Tissue: Any tissue of a plant in planta or in culture. This term is intended to include a whole plant, plant cell, plant organ, protoplast, cell culture, or any group of plant cells organized into a structural and functional unit.

Positive-sense inhibition: A type of gene regulation based on cytoplasmic inhibition of gene expression due to the presence in a cell of an RNA molecule substantially homologous to at least a portion of the mRNA being translated.

Promoter: The 5′-flanking, non-coding sequence substantially adjacent a coding sequence which is involved in the initiation of transcription of the coding sequence.

Protoplast: An isolated plant or bacterial cell without some or all of its cell wall.

Recombinant Plant Viral Nucleic Acid: Plant viral nucleic acid which has been modified to contain non-native nucleic acid sequences. These non-native nucleic acid sequences may be from any organism or purely synthetic, however, they may also include nucleic acid sequences naturally occurring in the organism into which the recombinant plant viral nucleic acid is to be introduced.

Recombinant Plant Virus: A plant virus containing the recombinant plant viral nucleic acid.

Subgenomic Promoter: A promoter of a subgenomic mRNA of a viral nucleic acid.

Substantial Sequence Homology: Denotes nucleotide sequences that are substantially functionally equivalent to one another. Nucleotide differences between such sequences having substantial sequence homology will be de minimis in affecting function of the gene products or an RNA coded for by such sequence.

Systemic Infection: Denotes infection throughout a substantial part of an organism including mechanisms of spread other than mere direct cell inoculation but rather including transport from one infected cell to additional cells either nearby or distant.

Transposon: A nucleotide sequence such as a DNA or RNA sequence which is capable of transferring location or moving within a gene, a chromosome or a genome.

Transgenic plant: A plant which contains a foreign nucleotide sequence inserted into either its nuclear genome or organellar genome.

Transcription: Production of an RNA molecule by RNA polymerase as a complementary copy of a DNA sequence or subgenomic mRNA.

Vector: A self-replicating RNA or DNA molecule which transfers an RNA or DNA segment between cells, such as bacteria, yeast, plant, or animal cells.

Virus: An infectious agent composed of a nucleic acid which may or may not be encapsidated in a protein. A virus may be a mono-, di-, tri-, or multi-partite virus, as described above.

In preferred embodiments, the present invention provides for the infection of a plant host by a recombinant plant virus containing a recombinant plant viral nucleic acid or by the recombinant plant viral nucleic acid which contains one or more non-native nucleic acid sequences which are subsequently transcribed or expressed in the infected tissues of the plant host. The product of the coding sequences may be recovered from the plant, produce a phenotypic trait in the plant, effect biochemical pathways within the plant or effect endogenous gene expression within the plant.

The present invention has a number of advantages. The instant invention allows practitioners to determine the function of a nucleic acid sequence which has been heretofore unknown.

The chimeric genes and vectors and recombinant plant viral nucleic acids used in this invention are constructed using techniques well known in the art. Suitable techniques have been described in Sambrook et al. (2nd ed.), Cold Spring Harbor Laboratory, Cold Spring Harbor (1982, 1989); Methods in Enzymol. (Vols. 68, 100, 101, 118, and 152-155) (1979, 1983, 1986 and 1987); and DNA Cloning, D. M. Clover, Ed., IRL Press, Oxford (1985). Medium compositions have been described by Miller, J., Experiments in Molecular Genetics, Cold Spring Harbor Laboratory, New York (1972), as well as the references previously identified, all of which are incorporated herein by reference. DNA manipulations and enzyme treatments are carried out in accordance with manufacturers' recommended procedures in making such constructs.

An important feature of the present invention is the use of recombinant plant viral nucleic acids which are capable of replication, local and/or systemic spread in a compatible plant host, and which contain one or more non-native subgenomic promoters which are capable of transcribing or expressing adjacent nucleic acid sequences in the plant host. The recombinant plant viral nucleic acids may be further modified to delete all or part of the native coat protein coding sequence and to contain a non-native coat protein coding sequence under control of the native or one of the non-native subgenomic promoters, or put the native coat protein coding sequence under the control of a non-native plant viral subgenomic promoter. The recombinant plant viral nucleic acids have substantial sequence homology to plant viral nucleotide sequences. A partial listing of suitable viruses is described, infra. The nucleotide sequence may be or may be derived from an RNA, DNA, cDNA or a chemically synthesized RNA or DNA.

The first step in producing recombinant plant viral nucleic acids according to this particular embodiment for use in the present invention is to modify the nucleotide sequences of the plant viral nucleotide sequence by known conventional techniques such that one or more non-native subgenomic promoters are inserted into the plant viral nucleic acid without destroying the biological function of the plant viral nucleic acid. The subgenomic promoters are capable of transcribing or expressing adjacent nucleic acid sequences in a plant host infected by the recombination plant viral nucleic acid or recombinant plant virus. The native coat protein coding sequence may be deleted in some embodiments, placed under the control of a non-native subgenomic promoter in other embodiments, or retained in a further embodiment. If it is deleted or otherwise inactivated, a non-native coat protein gene is inserted under control of one of the non-native subgenomic promoters, or optionally under control of the native coat protein gene subgenomic promoter. The non-native coat protein is capable of encapsidating the recombinant plant viral nucleic acid to produce a recombinant plant virus. Thus, the recombinant plant viral nucleic acid contains a coat protein coding sequence, which may be native or a normative coat protein coding sequence, under control of one of the native or non-native subgenomic promoters. The coat protein is involved in the systemic infection of the plant host.

Some of the viruses which meet this requirement, and therefore have been shown to be suitable for use according to the methods of the present invention, include viruses from the tobamovirus group such as Tobacco Mosaic virus (TMV), Ribgrass Mosaic Virus (RGM), Cowpea Mosaic virus (CMV), Alfalfa Mosaic virus (AMV), Cucumber Green Mottle Mosaic virus watermelon strain (CGMMV-W) and Oat Mosaic virus (OMV) and viruses from the brome mosaic virus group such as Brome Mosaic virus (BMV), broad bean mottle virus and cowpea chlorotic mottle virus. Additional suitable viruses include Rice Necrosis virus (RNV), and geminiviruses such as Tomato Golden Mosaic virus (TGMV), Cassava Latent virus (CLV) and Maize Streak virus (MSV). Each of these groups of suitable viruses is characterized below. However, the invention should not be construed as limited to using these particular viruses, but rather the method of the present invention is contemplated to include all plant viruses at a minimum.

### TOBAMOVIRUS GROUP

Tobacco Mosaic virus (TMV) is a member of the Tobamoviruses. The TMV virion is a tubular filament, and comprises coat protein sub-units arranged in a single right-handed helix with the single-stranded RNA intercalated between the turns of the helix. TMV infects tobacco as well as other plants. TMV is transmitted mechanically and may remain infective for a year or more in soil or dried leaf tissue.

The TMV virions may be inactivated by subjection to an environment with a pH of less than 3 or greater than 8, or by formaldehyde or iodine. Preparations of TMV may be obtained from plant tissues by (NH₄)₂SO₄ precipitation, followed by differential centrifugation.

The TMV single-stranded RNA genome is about 6400 nucleotides long, and is capped at the 5′-end but not polyadenylated. The genomic RNA can serve as mRNA for protein of a molecular weight of about 130,000 (130K) and another produced by read-through of molecular weight about 180,000 (180K). However, it cannot function as a messenger for the synthesis of coat protein. Other genes are expressed during infection by the formation of monocistronic, 3′-coterminal subgenomic mRNAs, including one (LMC) encoding the 17.5K coat protein and another (12) encoding a 30K protein. The 30K protein has been detected in infected protoplasts as described in Miller, J., Virology 132:53-60 (1984), and it is involved in the cell-to-cell transport of the virus in an infected plant as described by Deom et al., Science 237:389 (1987). The functions of the two large proteins are unknown, however, they are thought to function in RNA replication and transcription.

Several double-stranded RNA molecules, including double-stranded RNAs corresponding to the genomic, I₂ and LMC RNAs, have been detected in plant tissues infected with TMV. These RNA molecules are presumably intermediates in genome replication and/or mRNA synthesis processes which appear to occur by different mechanisms.

TMV assembly apparently occurs in plant cell cytoplasm, although it has been suggested that some TMV assembly may occur in chloroplasts since transcripts of ctDNA have been detected in purified TMV virions. Initiation of TMV assembly occurs by interaction between ring-shaped aggregates (“discs”) of coat protein (each disc consisting of two layers of 17 subunits) and a unique internal nucleation site in the RNA; a hairpin region about 900 nucleotides from the 3′-end in the common strain of TMV. Any RNA, including subgenomic RNAs containing this site, may be packaged into virions. The discs apparently assume a helical form on interaction with the RNA, and assembly (elongation) then proceeds in both directions (but much more rapidly in the 3′- to 5′-direction from the nucleation site).

Another member of the Tobamoviruses, the Cucumber Green Mottle Mosaic virus watermelon strain (CGMMV-W) is related to the cucumber virus. Nozu et al., Virology 45:577 (1971). The coat protein of CGMMV-W interacts with RNA of both TMV and CGMMV to assemble viral particles in vitro. Kurisu et al., Virology 70:214 (1976).

Several strains of the tobamovirus group are divided into two subgroups, on the basis of the location of the assembly of origin. Subgroup I, which includes the vulgare, OM, and tomato strain, has an origin of assembly about 800-1000 nucleotides from the 3′-end of the RNA genome, and outside the coat protein cistron. Lebeurier et al., Proc. Natl. Acad. Sci. USA 74:149 (1977); and Fukuda et al., Virology 101:493 (1980). Subgroup II, which includes CGMMV-W and compea strain (Cc) has an origin of assembly about 300-500 nucleotides from the 3′-end of the RNA genome and within the coat-protein cistron. The coat protein cistron of CGMMV-W is located at nucleotides 176-661 from the 3′-end. The 3′ noncoding region is 175 nucleotides long. The origin of assembly is positioned within the coat protein cistron. Meshi et al., Virology 127:54 (1983).

### BROME MOSAIC VIRUS GROUP

Brome Mosaic virus (BMV) is a member of a group of tripartite, single-stranded, RNA-containing plant viruses commonly referred to as the bromoviruses. Each member of the bromoviruses infects a narrow range of plants. Mechanical transmission of bromoviruses occurs readily, and some members are transmitted by beetles. In addition to BV, other bromoviruses include broad bean mottle virus and cowpea chlorotic mottle virus.

Typically, a bromovirus virion is icosahedral, with a diameter of about 26 μm, containing a single species of coat protein. The bromovirus genome has three molecules of linear, positive-sense, single-stranded RNA, and the coat protein mRNA is also encapsidated. The RNAs each have a capped 5′-end, and a tRNA-like structure (which accepts tyrosine) at the 3′-end. Virus assembly occurs in the cytoplasm. The complete nucleotide sequence of BMV has been identified and characterized as described by Ahlquist et al., J. Mol. Biol. 153:23 (1981).

### RICE NECROSIS VIRUS

Rice Necrosis virus is a member of the Potato Virus Y Group or Potyviruses. The Rice Necrosis virion is a flexuous filament comprising one type of coat protein (molecular weight about 32,000 to about 36,000) and one molecule of linear positive-sense single-stranded RNA. The Rice Necrosis virus is transmitted by Polymyxa oraminis (a eukaryotic intracellular parasite found in plants, algae and fungi).

### GEMNIVIRUSES

Geminiviruses are a group of small, single-stranded DNA-containing plant viruses with virions of unique morphology. Each virion consists of a pair of isometric particles (incomplete icosahedral), composed of a single type of protein (with a molecular weight of about 2.7-3.4×10⁴). Each geminivirus virion contains one molecule of circular, positive-sense, single-stranded DNA. In some geminiviruses (i.e., Cassava latent virus and bean golden mosaic virus) the genome appears to be bipartite, containing two single-stranded DNA molecules.

### POTYVIRUSES

Potyviruses are a group of plant viruses which produce polyprotein. A particularly preferred potyvirus is tobacco etch virus (TEV). TEV is a well characterized potyvirus and contains a positive-strand RNA genome of 9.5 kilobases encoding for a single, large polyprotein that is processed by three virus-specific proteinases. The nuclear inclusion protein “a” proteinase is involved in the maturation of several replication-associated proteins and capsid protein. The helper component-proteinase (HC-Pro) and 35-kDa proteinase both catalyze cleavage only at their respective C-termini. The proteolytic domain in each of these proteins is located near the C-terminus. The 35-kDa proteinase and HC-Pro derive from the N-terminal region of the TEV polyprotein.

Other particularly useful viruses according to some embodiments of the present invention feature viruses which are associated with animal hosts. Some of these viruses are discussed, infra.

### ALPHAVIRUSES

The alphaviruses are a genus of the viruses of the family Togaviridae. Almost all of the members of this genus are transmitted by mosquitoes, and may cause diseases in man or animals. Some of the alphaviruses are grouped into three serologicallly defined complexes. The complex-specific antigen is associated with the E1 protein of the virus, and the species-specific antigen is associated with the E2 protein of the virus.

The Semliki Forest virus complex includes Bebaru virus, Chikungunya Fever virus, Getah virus, Mayaro Fever virus, O'nyongnyong Fever virus, Ross River virus, Sagiyama virus, Semliki Forest virus and Una virus. The Venezuelan Equine Encephalomyelitis virus complex includes Cabassou virus, Everglades virus, Mucambo virus, Pixuna virus and Venezuelan Equine Encephalomyelitis virus. The Western Equine Encephalomyelitis virus complex includes Aura virus, Fort Morgan virus, Highlands J virus, Kyzylagach virus, Sindbis virus, Western Equine Encephalomyelitis virus and Whataroa virus.

The alphaviruses contain an icosahedral nucleocapsid consisting of 180 copies of a single species of capsid protein complexed with a plus-stranded mRNA. The alphaviruses mature when preassembled nucleocapsid is surrounded by a lipid envelope containing two virus-encoded integral membrane glycoproteins, called E1 and E2. The envelope is acquired when the capsid, assembled in the cytoplasm, buds through the plasma membrane. The envelope consists of a lipid bilayer derived from the host cell.

The mRNA encodes a glycoprotein which is cotranslationally cleaved into nonstructural proteins and structural proteins. The 3′ one-third of the RNA genome consists of a 26S mRNA which encodes for the capsid protein and the E3, E2, K6 and E1 glycoproteins. The capsid is cotranslationally cleaved from the E3 protein. It is hypothesized that the amino acid triad of His, Asp and Ser at the COOH terminus of the capsid protein comprises a serine protease responsible for cleavage. Hahn et al., Proc. Natl. Acad. Sci. USA 82:4648 (1985). Cotranslational cleavage also occurs between E2 and K proteins. Thus, two proteins PE2 which consists of E3 and E2 prior to cleavage and an E1 protein comprising K6 and E1 are formed. These proteins are cotranslationally inserted into the endoplasmic reticulum of the host cell, glycosylated and transported via the Golgi apparatus to the plasma membrane where they can be used for budding. At the point of virion maturation the E3 and E2 proteins are separated. The E1 and E2 proteins are incorporated into the lipid envelope.

It has been suggested that the basic amino-terminal half of the capsid protein stabilizes the interaction of capsid with genomic RNA or interacts with genomic RNA to initiate a encapsidation, Strauss et al., in the Togaviridae and Flaviviridae, Ed. S. Schlesinger & M. Schlesinger, Plenum Press, New York, pp. 35-90 (1980). These suggestions imply that the origin of assembly is located either on the unencapsidated genomic RNA or at the amino-terminus of the capsid protein. It has been suggested that E3 and K6 function as signal sequences for the insertion of PE2 and E1, respectively, into the endoplasmic reticulum.

Work with temperature sensitive mutants of alphaviruses has shown that failure of cleavage of the structural proteins results in failure to form mature virions. Lindquist et al., Virology 151:10 (1986) characterized a temperature sensitive mutant of Sindbis virus, tₛ 20. Temperature sensitivity results from an A-U change at nucleotide 9502. The tₛ lesion present cleavage of PE2 to E2 and E3 and the final maturation of progeny virions at the nonpermissive temperature. Hahn et al., supra, reported three temperature sensitive mutations in the capsid protein which prevents cleavage of the precursor polyprotein at the nonpermissive temperature. The failure of cleavage resulted in no capsid formation and very little envelope protein.

Defective interfering RNAs (DI particles) of Sindbis virus are helper-dependent eletion mutants which interfere specifically with the replication of the homologous standard virus. Perrault, J., Microbiol. Immunol. 93:151 (1981). DI particles have been found to be functional vectors for introducing at least one foreign gene into cells. Levis, R., Proc. Natl. Acad. Sci. USA 84:4811 (1987).

It has been found that it is possible to replace at least 1689 internal nucleotides of a DI genome with a foreign sequence and obtain RNA that will replicate and be encapsidated. Deletions of the DI genome do not destroy biological activity. The disadvantages of the system are that DI particles undergo apparently random rearrangements of the internal RNA sequence and size alterations. Monroe et al., J. Virology 49:865 (1984). Expression of a gene inserted into the internal sequence is not as high as expected. Levis et al., supra, found that replication of the inserted gene was excellent but translation was low. This could be the result of competition with whole virus particles for translation sites and/or also from disruption of the gene due to rearrangement through several passages.

Two species of mRNA are present in alphavirus-infected cells: A 42S mRNA region, which is packaged into nature virions and functions as the message for the nonstructural proteins, and a 26S mRNA, which encodes the structural polypeptides. the 26S mRNA is homologous to the 3′ third of the 42S mRNA. It is translated into a 130K polyprotein that is cotranslationally cleaved and processed into the capsid protein and two glycosylated membrane proteins, E1 and E2.

The 26S mRNA of Eastern Equine Encephalomyelitis (EEE) strain 82V-2137 was cloned and analyzed by Chang et al., J. Gen. Virol. 68:2129 (1987). The 26S mRNA region encodes the capsid proteins, E3, E2, 6K and E1. The amino terminal end of the capsid protein is thought to either stabilize the interaction of capsid with mRNA or to interact with genomic RNA to initiate encapsidation.

Uncleaved E3 and E2 proteins called PE2 is inserted into the host endoplasmic reticulum during protein synthesis. The PE2 is thought to have a region common to at least five alphaviruses which interacts with the viral nucleocapsid during morphogenesis.

The 6K protein is thought to function as a signal sequence involved in translocation of the E1 protein through the membrane. The E1 protein is thought to mediate virus fusion and anchoring of the E1 protein to the virus envelope.

### RHINOVIRUSES

The rhinoviruses are a genus of viruses of the family Picomaviridae. The rhinoviruses are acid-labile, and are therefore rapidly inactivated at pH values of less than about 6. The rhinoviruses commonly infect the upper respiratory tract of mammals.

Human rhinoviruses are the major causal agents of the common cold, and many serotypes are known. Rhinoviruses may be propagated in various human cell cultures, and have an optimum growth temperature of about 33° C. Most strains of rhinoviruses are stable at or below room temperature and can withstand freezing. Rhinoviruses can be inactivated by citric acid, tincture of iodine or phenol/alcohol mixtures.

The complete nucleotide sequence of human rhinovirus 2 (HRV2) has been sequenced. The genome consists of 7102 nucleotides with a long open reading frame of 6450 nucleotides which is initiated 611 nucleotides from the 5′-end and stops 42 nucleotides from the poly(A) tract. Three capsid proteins and their cleavage cites have been identified.

Rhinovirus RNA is single-stranded and positive-sense. The RNA is not capped, but is joined at the 5′-end to a small virus-encoded protein, virion-protein genome-linked (VPg). Translation is presumed to result in a single polyprotein which is broken by proteolytic cleavage to yield individual virus proteins. An icosahedral viral capsid contains 60 copies each of 4 virus proteins VP1, VP2, VP3 and VP4 and surrounds the RNA genome. Medappa, K., Virology 44:259 (1971).

Analysis of the 610 nucleotides preceding the long open reading frame shows several short open reading frames. However, no function can be assigned to the translated proteins since only two sequences show homology throughout HRV2, HRV14 and the 3 sterotypes of poliovirus. These two sequences may be critical in the life cycle of the virus. They are a stretch of 16 bases beginning at 436 in HRV2 and a stretch of 23 bases beginning at 531 in HRV2. Cutting or removing these sequences from the remainder of the sequence for non-structural proteins could have an unpredictable effect upon efforts to assemble a mature virion.

The capsid proteins of HRV2: VP4, VP2, VP3 and VP1 begin at nucleotide 611, 818, 1601 and 2311, respectively. The cleavage point between VP1 and P2A is thought to be around nucleotide 3255. Skern et al., Nucleic Acids Research 13:2111 (1985).

Human rhinovirus type 89 (HRV89) is very similar to HRV2. It contains a genome of 7152 nucleotides with a single large open reading frame of 2164 condons. Translation begins at nucleotide 619 and ends 42 nucleotides before the poly(A) tract. The capsid structural proteins, VP4, VP2, VP3 and VP1 are the first to be translated. Translation of VP4 begins at 619. Cleavage cites occur at:

| | | |
|---|---|---|
| VP4/VP2 | 825 | determined |
| VP2/VP3 | 1627 | determined |
| VP3/VP1 | 2340 | determined |
| VP1/P2-A | 3235 | presumptive |

Duechler et al., Proc. Natl. Acad. Sci. USA 84:2605 (1987).

### POLIOVIRUSES

Polioviruses are the causal agents of poliomyelitis in man, and are one of three groups of enteroviruses. Enteroviruses are a genus of the family Picornaviridae (also the family of rhinoviruses). Most enteroviruses replicate primarily in the mammalian gastrointestinal tract, although other tissues may subsequently become infected. Many enteroviruses can be propagated in primarily cultures of human or monkey kidney cells and in some cell lines (e.g. HeLa, Vero, WI-e8). Inactivation of the enteroviruses may be accomplished with heat (about 50° C.), formaldehyde (3%), hydrochloric acid (0.1N) or chlorine (ca. 0.3-0.5 ppm free residual C1₂).

The complete nucleotide sequence of poliovirus PV2 (Sab) and PV3 (Sab) have been determined. They are 7439 and 7434 nucleotide in length, respectively. There is a single long open reading frame which begins more than 700 nucleotides from the 5′-end. Poliovirus translation produces a single polyprotein which is cleaved by proteolytic processing. Kitamura et al, Nature 291:547 (1981).

It is speculated that these homologous sequences in the untranslated regions play an essential role in viral replication such as:
1. viral-specific RNA synthesis;
2. viral-specific protein synthesis; and
3. packaging
   Toyoda, H. et al., J. Mol. Biol. 174:561 (1984).

The structures of the serotypes of poliovirus have a high degree of sequence homology. Their coding sequences code for the same proteins in the same order. Therefore, genes for structural proteins are similarly located. In PV1, PV2 and PV3, the polyprotein begins translation near the 750 nucleotide. The four structural proteins VP4, VP2, VP3 and VP1 begin at about 745, 960, 1790 and 2495, respectively, with VPI ending at about 3410. They are separated in vivo by proteolytic cleavage, rather than by stop/start codons.

### SIMIAN VIRUS 40

Simian virus 40 (SV40) is a virus of the genus Polyomavirus, and was originally isolated from the kidney cells of the rhesus monkey. The virus is commonly found, in its latent form, in such cells. Simian virus 40 is usually non-pathogenic in its natural host.

Simian virus 40 virions are made by the assembly of three structural proteins, VP1, VP2 and VP3. Girard et al., Biochem. Biophys. Res. Commun. 40:97 (1970); Prives et al., Proc. Natl. Acad. Sci. USA 71:302 (1974); and Jacobson et al., Proc. Natl. Acad. Sci. USA 73:2747 (1976). The three corresponding viral genes are organized in a partially overlapping manner. They constitute the late genes portion of the genome. Tooze, J., Molecular Biology of Tumor Viruses Appendix A The SV40 Nucleotide Sequence, 2nd Ed. Part 2, pp. 799-831 (1980), Cold Spring Harbor Laboratory, Cold Spring Harbor, N.Y. Capsid proteins VP2 and VP3 are encoded by nucleotides 545 to 1601 and 899 to 1601, respectively, and both are read in the same frame. VP3 is therefore a subset of VP2. Capsid protein VP1 is encoded by nucleotides 1488-2574. The end of the VP2-VP3 open reading frame therefore overlaps the VP1 by 113 nucleotides but is read in an alternative frame. Tooze, J., supra. Wychowski et al., J. Virology 61:3862 (1987).

### ADENOVIRUSES

Adenovirus type 2 is a member of the adenovirus family or adenovirus. This family of viruses are non-enveloped, icosahedral, linear, double-stranded DNA-containing viruses which infect mammals or birds.

The adenovirus virion consists of an icosahedral capsid enclosing a core in which the DNA genome is closely associated with a basic (arginine-rich) viral polypeptide VII. The capsid is composed of 252 capsomeres: 240 hexons (capsomers each surrounded by 6 other capsomers) and 12 pentons (one at each vertex, each surrounded by 5 ‘peripentonal’ hexons). Each penton consists of a penton base (composed of viral polypeptide III) associated with one (in mammalian adenoviruses) or two (in most avian adenoviruses) glycoprotein fibres (viral polypeptide IV). The fibres can act as haemagglutinins and are the sites of attachment of the virion to a host cell-surface receptor. The hexons each consist of three molecules of viral polypeptide II; they make up the bulk of the icosahedron. Various other minor viral polypeptides occur in the virion.

The adenovirus dsDNA genome is covalently linked at the 5′-end of each strand to a hydrophobic ‘terminal protein’, TP (molecular weight about 55,000 Da); the DNA has an inverted terminal repeat of different length in different adenoviruses. In most adenoviruses examined, the 5′-terminal residue is dCMP.

During its replication cycle, the virion attaches via its fibres to a specific cell-surface receptor, and enters the cell by endocytosis or by direct penetration of the plasma membrane. Most of the capsid proteins are removed in the cytoplasm. The virion core enters the nucleus, where the uncoating is completed to release viral DNA almost free of virion polypeptides. Virus gene expression then begins. The viral dsDNA contains genetic information on both strands. Early genes (regions E1a, E1b, E2a, E3, E4) are expressed before the onset of viral DNA replication. Late genes (regions L1, L2, L3, L4 and L5) are expressed only after the initiation of DNA synthesis. Intermediate genes (regions E2b and Iva₂) are expressed in the presence or absence of DNA synthesis. Region E1a encodes proteins involved in the regulation of expression of other early genes, and is also involved in transformation. The RNA transcripts are capped (with m⁷G⁵ppp⁵N) and polyadenylated in the nucleus before being transferred to the cytoplasm for translation.

Viral DNA replication requires the terminal protein, TP, as well as virus-encoded DNA polymerase and other viral and host proteins. TP is synthesized as an 80K precursor, pTP, which binds covalently to nascent replicating DNA strands. pTP is cleaved to the mature 55K TP late in virion assembly; possibly at this stage, pTP reacts with a dCTP molecule and becomes covalently bound to a dCMP residue, the 3′ OH of which is believed to act as a primer for the initiation of DNA synthesis. Late gene expression, resulting in the synthesis of viral structural proteins, is accompanied by the cessation of cellular protein synthesis, and virus assembly may result in the production of up to 10⁵ virions per cell.

In addition to the plant and animal viruses described above, viral expression system in bacteria and yeast cells may also be employed. See Munishkin et al., Nature 333(6172):473-5 (1988) and Priano et al., J. Mol. Biol. 271(3):299-310 (1997) for viral expression system in bacteria and Janda et al., Cell 72(6):961-70 (1993) and Ishikawa et al., J. Virol. 71(10):7781-90 (1997) for viral expression in yeast. The teachings of these references are incorporated herein by reference.

The nucleic acid of any suitable plant virus can be utilized to prepare a recombinant plant viral nucleic acid for use in the present invention, and the foregoing are only exemplary of such suitable plant viruses. The nucleotide sequence of the plant virus is modified, using conventional techniques, by the insertion of one or more subgenomic promoters into the plant viral nucleic acid. The subgenomic promoters are capable of functioning in the specific host plant. For example, if the host is tobacco, TMV, TEV, or other viruses containing subgenomic promoter may be utilized. The inserted subgenomic promoters should be compatible with the TMV nucleic acid and capable of directing transcription or expression of adjacent nucleic acid sequences in tobacco. The native coat protein gene could also be retained and a non-native nucleic acid sequence inserted within it to create a fusion protein.

The native or non-native coat protein gene is utilized in the recombinant plant viral nucleic acid. Whichever non-native nucleic acid is utilized may be positioned adjacent its natural subgenomic promoter or adjacent one of the other available subgenomic promoters. The non-native coat protein, as is the case for the native coat protein, is capable of encapsidating the recombinant plant viral nucleic acid and providing for systemic spread of the recombinant plant viral nucleic acid in the host plant. The coat protein is selected to provide a systemic infection in the plant host of interest. For example, the TMV-O coat protein provides systemic infection in N. benthamiana, whereas TMV-U1 coat protein provides systemic infection in N. tabacum.

The recombinant plant viral nucleic acid is prepared by cloning a viral nucleic acid. If the viral nucleic acid is DNA, it can be cloned directly into a suitable vector using conventional techniques. One technique is to attach an origin of replication to the viral DNA which is compatible with the cell to be transfected. If the viral nucleic acid is RNA, a full-length DNA copy of the viral genome is first prepared by well-known procedures. For example, the viral RNA is transcribed into DNA using reverse transcriptase to produce subgenomic DNA pieces, and a double-stranded DNA made using DNA polymerases. The cDNA is then cloned into appropriate vectors and cloned into a cell to be transfected. Alternatively, the cDNA's ligated into the vector may be directly transcribed into infectious RNA in vitro and inoculated onto the plant host. The CDNA pieces are mapped and combined in proper sequence to produce a full-length DNA copy of the viral RNA genome, if necessary. DNA sequences for the subgenomic promoters, with or without a coat protein gene, are then inserted into the nucleic acid at non-essential sites, according to the particular embodiment of the invention utilized. Non-essential sites are those that do not affect the biological properties of the plant viral nucleic acid. Since the RNA genome is the infective agent, the cDNA is positioned adjacent a suitable promoter so that the RNA is produced in the production cell. The RNA is capped using conventional techniques, if the capped RNA is the infective agent. In addition, the capped RNA can be packaged in vitro with added coat protein from TMV to make assembled virions. These assembled virions can then be used to inoculate plants or plant tissues.

Alternatively, an uncapped RNA may also be employed in the embodiments of the present invention. Contrary to the practiced art in scientific literature and in issued patent (Ahlquist et al., U.S. Pat. No. 5,466,788), uncapped transcripts for virus expression vectors are infective on both plants and in plant cells. Capping is not a prerequisite for establishing an infection of a virus expression vector in plants, although capping increases the efficiency of infection. In addition, nucleotides may be added between the transcription start site of the promoter and the start of the cDNA of a viral nucleic acid to construct an infectious viral vector. One or more nucleotides may be added. In a preferred embodiment of the present invention, the inserted nucleotide sequence contains a G at the 5′-end. In a particularly preferred embodiment, the inserted nucleotide sequence is GNN, GTN, or their multiples, (GNN)ₓ or (GTN)ₓ.

Another feature of these recombinant plant viral nucleic acids useful in the present invention is that they further comprise one or more nucleic acid sequences capable of being transcribed in the plant host. These nucleic acid sequences may be native nucleic acid sequences which occur in the host organism or they may be non-native nucleic acid sequences which do not normally occur in the host organism. The nucleic acid sequence is placed adjacent one of the non-native viral subgenomic promoters and/or the native coat protein gene promoter depending on the particular embodiment used. The nucleic acid is inserted by conventional techniques, or the nucleic acid sequence can be inserted into or adjacent the native coat protein coding sequence such that a fusion protein is produced. The nucleic acid sequence which is transcribed may be transcribed as an RNA which is capable of regulating the expression of a phenotypic trait by an anti-sense or a positive-sense mechanism. Alternatively, the nucleic acid sequence in the recombinant plant viral nucleic acid may be transcribed and translated in the plant host to produce a phenotypic trait. The nucleic acid sequence(s) may also code for the expression of more than one phenotypic trait. The recombinant plant viral nucleic acid containing the nucleic acid sequence is constructed using conventional techniques such that the nucleic acid sequence(s) are in proper orientation to whichever viral subgenomic promoter is utilized.

A double-stranded DNA of the recombinant plant viral nucleic acid or a complementary copy of the recombinant plant viral nucleic acid is cloned into the cell to be transfected. If the viral nucleic acid is a RNA molecule, the nucleic acid (cDNA) is first attached to a promoter which is compatible with the production cell. The recombinant plant viral nucleic acid can then be cloned into any suitable vector which is compatible with the production cell. In this manner, only RNA copies of the chimeric nucleotide sequence are produced in the production cell. For example, the CaMV promoter can be used when plant cells are to be transfected. Alternatively, the recombinant plant viral nucleic acid is inserted in a vector adjacent a promoter which is compatible with the production cell. If the viral nucleic acid is a DNA molecule, it can be cloned directly into a production cell by attaching it to an origin of replication which is compatible with the cell to be transfected. In this manner, DNA copies of the chimeric nucleotide sequence are produced in the transfected cell.

A further alternative when creating the recombinant plant viral nucleic acid is to prepare more than one nucleic acid (i.e., to prepare the nucleic acids necessary for a multipartite viral vector construct). In this case, each nucleic acid would require its own origin of assembly. Each nucleic acid could be prepared to contain a subgenomic promoter and a non-native nucleic acid.

Alternatively, the insertion of a non-native nucleic acid into the nucleic acid of a monopartite virus may result in the creation of two nucleic acids (i.e., the nucleic acid necessary for the creation of a bipartite viral vector). This would be advantageous when it is desirable to keep the replication and transcription or expression of the nucleic acid of interest separate from the replication and translation of some of the coding sequences of the native nucleic acid. Each nucleic acid would have to have its own origin of assembly.

The host can be infected with the recombinant plant virus by conventional techniques. Suitable techniques include, but are not limited to, leaf abrasion, abrasion in solution, high velocity water spray and other injury of a host as well as imbibing host seeds with water containing the recombinant plant virus. More specifically, suitable techniques include:
(a) Hand Inoculations. Hand inoculations of the encapsidated vector are performed using a neutral pH, low molarity phosphate buffer, with the addition of celite or carborundum (usually about 1%). One to four drops of the preparation is put onto the upper surface of a leaf and gently rubbed.
(b) Mechanized Inoculations of Plant Beds. Plant bed inoculations are performed by spraying (gas-propelled) the vector solution into a tractor-driven mower while cutting the leaves. Alternatively, the plant bed is mowed and the vector solution sprayed immediately onto the cut leaves.
(c) High Pressure Spray of Single Leaves. Single plant inoculations can also be performed by spraying the leaves with a narrow, directed spray (50 psi, 6-12 inches from the leaf) containing approximately 1% carborundum in the buffered vector solution.
(d) Vacuum Infiltration. Inoculations may be accomplished by subjecting the host organism to a substantially vacuum pressure environment in order to facilitate infection.
(e) High Speed Robotics Inoculation. Especially applicable when the organism is a plant, individual organisms may be grown in mass array such as in microtiter plates. Machinery such as robotics may then be used to transfer the nucleic acid of interest.

An alternative method for introducing a recombinant plant viral nucleic acid into a plant host is a technique known as agroinfection or Agrobacterium-mediated transformation (sometimes called Agro-infection) as described by Grimsley et al., Nature 325:177 (1987). This technique makes use of a common feature of Agrobacterium which colonizes plants by transferring a portion of their DNA (the T-DNA) into a host cell, where it becomes integrated into nuclear DNA. The T-DNA is defined by border sequences which are 25 base pairs long, and any DNA between these border sequences is transferred to the plant cells as well. The insertion of a recombinant plant viral nucleic acid between the T-DNA border sequences results in transfer of the recombinant plant viral nucleic acid to the plant cells, where the recombinant plant viral nucleic acid is replicated, and then spreads systemically through the plant. Agro-infection has been accomplished with potato spindle tuber viroid (PSTV) (Gardner et al., Plant Mol. Biol. 6:221 (1986); CaV (Grimsley et al., Proc. Natl. Acad. Sci. USA 83:3282 (1986)); MSV (Grimsley et al., Nature 325:177 (1987)), and Lazarowitz, S., Nucl. Acids Res. 16:229 (1988)) digitaria streak virus (Donson et al., Virology 162:248 (1988)), wheat dwarf virus (Hayes et al., J. Gen. Virol. 69:891 (1988)) and tomato golden mosaic virus (TGMV) (Elmer et al., Plant Mol. Biol. 10:225 (1988) and Gardiner et al., EMBO J. 7:899 (1988)). Therefore, agro-infection of a susceptible plant could be accomplished with a virion containing a recombinant plant viral nucleic acid based on the nucleotide sequence of any of the above viruses. Particle bombardment or electrosporation or any other methods known in the art may also be used.

Infection may also be attained by placing a selected nucleic acid sequence into an organism such as E. coli, or yeast, either integrated into the genome of such organism or not and then applying the organism to the surface of the host organism. Such a mechanism may thereby produce secondary transfer of the selected nucleic acid sequence into the host organism. This is a particularly practical embodiment when the host organism is a plant. Likewise, infection may be attained by first packaging a selected nucleic acid sequence in a pseudovirus. Such a method is described in WO 94/10329, the teachings of which are incorporated herein by reference. Though the teachings of this reference may be specific for bacteria, those of skill in the art will readily appreciate that the same procedures could easily be adapted to other organisms.

Those of skill in the art will readily understand that there are many methods to determine the function of a nucleic acid once expression in a host, such as a plant is attained. In one embodiment the function of a nucleic acid may be determined by complementation analysis. That is, the function of the nucleic acid of interest may be determined by observing the endogenous gene or genes whose function is replaced or augmented by introducing the nucleic acid of interest. A discussion of this principle is provided by Napoli et al., The Plant Cell 2:279-289 (1990) which is incorporated herein by reference. Further teachings in these regards are provided by WO 97/42210, the disclosure of which is also incorporated herein by reference. In a second embodiment, the function of a nucleic acid may be determined by analyzing the biochemical alterations in the accumulation of substrates or products from enzymatic reactions according to any one of the means known by those skilled in the art. In a third embodiment, the function of a nucleic acid may be determined by observing phenotypic changes in the host by methods including morphological, macroscopic or microscopic analysis. In a fourth embodiment, the function of a nucleic acid may be determined by observing the change in biochemical pathways which may be modified in the host as a result of the local and/or systemic expression of the non-native nucleic acids. In a fifth embodiment, the function of a nucleic acid may be determined utilizing techniques known by those skilled in the art to observe inhibition of gene expression in the cytoplasm of cells as a result of expression of the non-native nucleic acid.

A particularly useful way to determine gene function is by observing the phenotype in a whole plant when a particular gene function has been silenced. Useful phenotypic traits in plant cells which may be observed microscopically, macroscopically or by other methods include, but are not limited to, improved tolerance to herbicides, improved tolerance to extremes of heat or cold, drought, salinity or osmotic stress; improved resistance to pests (insects, nematodes or arachnids) or diseases (fungal, bacterial or viral) production of enzymes or secondary metabolites; male or female sterility; dwarfness; early maturity; improved yield, vigor, heterosis, nutritional qualities, flavor or processing properties, and the like. Other examples include the production of important proteins or other products for commercial use, such as lipase, melanin, pigments, alkaloids, antibodies, hormones, pharmaceuticals, antibiotics and the like. Another useful phenotypic trait is the production of degradative or inhibitory enzymes, such as are utilized to prevent or inhibit root development in malting barley or that determine response or non-response to a systemically administered drug in a human. The phenotypic trait may also be a secondary metabolite whose production is desired in a bioreactor.

Another particularly useful means to determine function of nucleic acids transfected into a host is to observe the effects of gene silencing. Traditionally, functional gene knockout has been achieved following inactivation due to insertion of transposable elements or random integration of T-DNA into the chromosome, followed by characterization of conditional, homozygous-recessive mutants obtained upon backcrossing. Some teachings in these regards are provided by WO 97/42210 which is herein incorporated by reference. As an alternative to traditional knockout analysis, an EST/DNA library from an organism, for example Arabidopsis thaliana, may be assembled into a plant viral transcription plasmid. The DNA sequences in the transcription plasmid library may then be introduced into plant cells as part of a functional RNA virus which post-transcriptionally silences the homologous target gene. The EST/DNA sequences may be introduced into a plant viral vector in either the plus or minus sense orientation, and the orientation can be either directed or random based on the cloning strategy. A high-throughput, automated cloning scheme based on robotics may be used to assemble and characterize the library. In addition, double stranded RNA may also be an effective stimulator of gene silencing/co-suppression in transgenic plant. Gene silencing/co-suppression of plant genes may be induced by delivering an RNA capable of base pairing with itself to form double stranded regions. This approach could be used with any plant or non-plant gene to assist in the identification of the function of a particular gene sequence.

A particularly troublesome problem with gene silencing in plant hosts is that many plant genes exist in a multigene family. Therefore, effective silencing of a gene function may be especially problematic. According to the present invention, however, nucleic acids may be inserted into the genome to effectively silence a particular gene function or to silence the function of a multigene family. It is presently believed that about 20% of plant genes exist in multigene families. A single nucleotide sequence of about 20 to 100 or more bases having about 70% or more homology to a gene may silence an entire plant gene family having two or more homologous genes.

A detailed discussion of some aspects of the “gene silencing” effect is provided in co-pending U.S. patent application Ser. No. 08/260,546 (WO95/34668 published Dec. 21, 1995) the disclosure of which is incorporated herein by reference. RNA can reduce the expression of a target gene through inhibitory RNA interactions with target mRNA that occur in the cytoplasm and/or the nucleus of a cell.

Full-length cDNAs may be accessed from public and private repositories or extracted from field samples for insertion of unknown open reading frames into viral vectors for expression of nucleic acids in the host organism and thereby utilized as an alternative to antisense gene knockout. This technology may be implemented by PCR amplification and cloning of all cDNAs that do not share homology with gene sequences in public and or private databases. The cDNAs may be expressed in plants transfected with one or more plant viral vectors for subsequent analysis of novel phenotype of the whole plant (biochemical and morphological). Selected cDNA sequences from maize, rice, soybean canola and other crop species may be used to assemble the cDNA libraries. This method may thus be used to search for useful dominant gene phenotypes from novel cDNA libraries through the gene expression.

An EST/cDNA library from an organism such as Arabidopsis thaliana may be assembled into a plant viral transcription plasmid background. The cDNA sequences in the transcription plasmid library can then be introduced into plant cells as cytoplasmic RNA in order to post-transcriptionally silence the endogenous genes. The EST/cDNA sequences may be introduced into the plant viral transcription plasmid in either the plus or anti-sense orientation (or both), and the orientation can be either directed or random based on the cloning strategy. A high-throughput, automated cloning strategy using robotics can be used to assemble the library. The EST clones can be inserted behind a duplicated subgenomic promoter such that they are represented as subgenomic transcripts during viral replication in plant cells. Alternatively, the EST/cDNA sequences can be inserted into the genomic RNA of a plant viral vector such that they are represented as genomic RNA during the viral replication in plant cells. The library of EST clones is then transcribed into infectious RNA and inoculated onto individual platelets of Arabidopsis thaliana (or other plant species). The viral RNA containing the EST/cDNA sequences contributed from the original library are now present in a sufficiently high concentration in the cytoplasm such that they cause post-transcriptional gene silencing of the endogenous plant-gene homologs. Since the replication mechanism of the virus produces both sense and antisense RNA sequences, the orientation of the EST/cDNA insert is normally irrelevant in terms of producing the desired gene-silenced phenotype in the tissue. Partial cDNA sequences cloned into a plant viral vector in the sense orientation have previously been shown to also confer a gene silencing phenotype (Kumagai et al., Proc. Natl. Acad. Sci. USA 92:1679 (1995)), the teachings of which are incorporated herein by reference. The actual mechanism of gene silencing has not been fully determined. This phenomenon may be similar to the gene silencing via cosuppression observed in transgenic plants.

The plant tissue may then be taken for sophisticated biochemical analysis in order to determine which metabolic pathway has been affected by the EST/DNA gene silencing, and in particular, which steps in a given metabolic pathway have been affected by the EST/DNA gene silencing. Biochemical analysis may be done, for example, in a high-throughput, fully automated fashion using robotics. Suitable biochemical analysis may include MALDI-TOF, LC/MS, GC/MS, two-dimensional IEF/SDS-PAGE, ELISA or other methods of analyses. The clones in the EST/plant viral vector library may then be functionally classified based on metabolic pathway affected or visual/selectable phenotype produced in the plant. This process enables the rapid determination of gene function for unknown EST/DNA sequences of plant origin. Furthermore, this process can be used to rapidly confirm function of full-length DNA's of unknown gene function. Functional identification of unknown EST/DNA sequences in a plant library may then rapidly lead to identification of similar unknown sequences in expression libraries for other crop species based on sequence homology.

Large amounts of DNA sequence information is being generated in the public domain and may be entered into a relational database. Links may be made between sequences from various species predicted to carry out similar biochemical or regulatory functions. Links may also be generated between predicted enzymatic activities and visually displayed biochemical and regulatory pathways. Likewise, links may be generated between predicted enzymatic or regulatory activity and known small molecule inhibitors, activators, substrates or substrate analogs. Phenotypic data from expression libraries expressed in transfected hosts maybe automatically linked within such a relational database. Genes with similar predicted roles of interest in other crop plants or crop plant pests may thereby be more rapidly discovered.

A complete classification scheme of gene functionality for a fully sequenced eukaryotic organism has been established for yeast. This classification scheme may be modified for plants and divided into the appropriate categories. Such organizational structure may be utilized to rapidly identify herbicide target loci which may confer dominant lethal phenotypes, and thereby is useful in helping to design rational herbicide programs.

A second aspect of the present invention is a method of silencing endogenous genes in a host by introducing nucleic acids into the host by way of a viral nucleic acid suitable to produce the local and systemic expression of the nucleic acid of interest. In one embodiment, the host is a plant, but those skilled in the art will understand that other hosts may also be utilized. This method utilizes the principle of post-transcription gene silencing of the endogenous host gene homolog as described above. Since the replication mechanism produces both sense and anti-sense RNA sequences as disclosed above, the orientation of the non-native nucleic acid insert is not crucial to providing gene silencing.

More information describing some aspects of the “gene silencing” effect is provided in co-pending U.S. patent application Ser. No. 08/260,546 (WO 95/34668 published Dec. 21, 1995) the disclosure of which is incorporated herein by reference. RNA can reduce the expression of a target gene through inhibitory RNA interactions with target mRNA that occur in the cytoplasm and/or the nucleus of a cell.

Silencing of endogenous genes can be achieved with homologous (but not identical) sequences from distant plant species. For example, the Nicotiana benthamiana gene for phytoene desaturase (PDS) may be silenced by transfection with a partial tomato cDNA for PDS (cloned in either the positive or antisense orientation). The tomato PDS cDNA is 92% homologous at the nucleotide level yet is still able to confer efficient gene silencing in an unrelated plant species (Kumagai et al., Proc. Natl. Acad. Sci. USA 92:1679 (1995)). Identification of EST/cDNA gene function in Arabidopsis thaliana could then be extrapolated to similar EST/cDNA sequences of unknown function that exist in other libraries (e.g., soybean, maize, rice, oilseed rape, etc.).

A third aspect of the present invention is a method for selecting desired functions of RNAs and proteins by the use of virus vectors to express libraries of nucleic acid sequence variants. Libraries of sequence variants may be generated by means of in vitro mutagenenisis and/or recombination. Rapid in vitro evolution can be used to improve virus-specific or protein-specific functions. In particular, plant RNA virus expression vectors may be used as tools to bear libraries containing variants of nucleic acid, genes from virus, plant or other sources, and to be applied to plants or plant cells such that the desired altered effects in the RNA or protein products can be determined, selected and improved. In a preferred embodiment, nucleic acid shuffling techniques may be employed to construct shuffled gene libraries. Random, semi-random or known sequences of virus origin may also be inserted in virus expression vectors between native virus sequences and foreign gene sequences, to increase the genetic stability of foreign genes in expression vectors as well as the translation of the foreign gene and the stability of the mRNA encoding the foreign gene in vivo. The desired function of RNA and protein may include the promoter activities, replication properties, translational efficiencies, movement properties (local and systemic), signaling pathway, or virus host range, among others. The desired function alteration can be identified by assaying infected plants and the nature of mutation can be determined by analysis of sequence variants in the virus vector.

Methods to increase the representation of gene sequences in virus expression libraries may also be achieved by bypassing the genetic bottleneck of propagation in E. coli. For example, in one of the preferred embodiments of the instant invention, cell-free methods may be used to clone sequence libraries or individual arrayed sequences into virus expression vectors and reconstruct an infectious virus, such that the final ligation product can be transcribed and the resulting RNA can be used for plant or plant cell inoculation/infection with the output being gene fimction discovery or protein production.

Techniques to screen sequence libraries can be introduced into RNA viruses or RNA virus vectors as populations or individuals in parallel to identify individuals with novel and augmented virus-encoded finctions in replication and virus movement, foreign gene sequence retention in vectors and proper folding, activity and expression of protein products, novel gene expression, effects on host metabolism, and resistance or susceptibility of plants to exogenous agents.

Variation in the sequence of a native virus gene(s) or heterologous nucleotide sequence(s) may be introduced into an RNA virus or an RNA virus expression vector by many methods as a means to screen a population of variants in batch or individuals in parallel for novel properties exhibited by the virus itself or conferred on the host plant or cell by the virus vector. Variant populations can be transfected as populations or individual clones into “host”: 1) protoplasts; 2) whole plants; or 3) inoculated leaves of whole plants and screened for various traits including protein expression (increase or decrease), RNA expression (increase or decrease), secondary metabolites or other host property gained or loss as a result of the virus infection.

For treatment of hosts with agents that result in cell death or down regulation in general metabolic function, a virus vector, which simultaneously expressed the green fluorescent protein (GFP) or other selectable marker gene and the variant sequence, is used to screen quantitatively for levels of resistance or sensitivity to the agent in question conferred upon the host by the variant sequence expressed from the viral vector. By quantitatively screening pools or individual infection events, those viruses containing unique variant sequences allowing sustained metabolic life of host are identified by fluorescence under long wave UV light. Those that do not confer this phenotype will fail to or poorly fluoresce. In this manner, high throughput screening in multi-well dishes in plate readers is possible where the average fluorescence of the well would be expressed as a ratio of the adsorption (measuring the cell mass) thereby giving a comparable quantitative value. This technique enables screening of populations or individuals followed by rescue of the sequence from virus vectors conferring desired trait by RT-PCR and re-screening of particular variant sequences in secondary screens.

The finctions of transcription factors or factors contributing to the signal transduction pathway of host cells are monitored by using specific proteomic, mRNA or metanomic traits to be assayed following transfection with a virus expression library. The contribution of a particular protein or product to a valuable trait may be known from the literature, but a new mode of enhanced or reduced expression could be identified by finding the factors that respond to cellular signals that in turn alter its particular expression. For example, transcription factors regulating the expression of defense proteins such as systemin peptides, or protease inhibitors could be identified by transfecting hosts with virus libraries and the expression of systemin or protease inhibitors or their RNAs be directly assayed. Conversely, the promoters responsible for expressing these genes could be genetically fused to the green fluorescent protein and introduced into hosts as transient expression constructs or into stable transformed host cells/tissues. The resulting cells would be transfected with viral vector libraries. Hosts now could be screened rapidly by following relative GFP expression following vector transfection. Likewise, coupling the transfecting of hosts with virus libraries with the treatment of plants with methyl jasmonate could identify sequences that reverse or enhance the gene induction events induced by this metabolite. This approach could be applied to other factors involved in promotion of higher biomass in plants such as Leafy or DET2. The expression of these factors could be directly assayed or via promoters genetically fused to GFP. This technique will enable screening of populations or individuals followed by rescue of the sequence from virus vectors conferring desired trait by RT-PCR and re-screening of particular variant sequences in secondary screens.

A fourth aspect of the present invention is a method for inhibiting an endogenous protease of a plant host comprising the step of treating the plant host with a compound which induces the production of an endogenous inhibitor of said protease. In a preferred embodiment, jasmonic acid may be used to treat the plant host to induce the production of an endogenous inhibitor of an endogenous protease. In another preferred embodiment, the treatment of the plant host with a compound results an increased representation of an exogenous nucleic acid or the protein product thereof. In particular, transgenic hosts expressing protease inhibitors may be used to decrease the degradation of proteins expressed by virus expression vectors. In a preferred embodiment, jasmonic acid may be used to treat plants infected with virus expression vectors to decrease the degradation of proteins expressed by virus expression vectors.

A fifth aspect of the present invention are genes and fragments thereof, nucleotide sequences, and gene products obtained by way of the method of the present invention. The present invention features expressing selected nucleotide sequences in a host organism such as, for example, a plant. Those of skill in the art will readily appreciate that the gene products of such nucleotide sequences may be isolated using techniques known to those skilled in the art. Such gene products may exhibit biological activity as pharmaceuticals, herbicides, and other similar functions.

The present invention is also directed to a method for identifying a gene function in a transgenic plant carrying a conditional lethal mutation in a gene. The method comprises of: (a) growing the plant under first permissive conditions; (b) exposing the plant from step (a) to restrictive conditions for a period of time of at least about one growth cycle; (c) shifting the plant from step (b) to second permissive conditions for a period of time of at least about one growth cycle; and (d) selecting a plant having a lethal mutation, thereby identifying a plant carrying a lethal mutation that is sensitive to the restrictive condition and essential for survival of the organism. The method further comprises after step (d), a step (e) complementing a transgenic plant carrying a recessive or dominant conditional lethal mutation by transfecting with a viral vector containing a functional copy of the mutated gene. The method further comprises after step (e), a step (f) isolating from said viral vector a gene correcting or complementing said mutation. The method further comprises after step (f), a step (g) selected from (i) identifying the function of said gene, (ii) identifying the product expressed by said gene, and (iii) sequencing said gene. In the method, the first permissive conditions include a complete growth medium for the plant tissue, plant cell or plant organ. The first permissive conditions also include a growth medium at low osmotic strength. The first permissive conditions further include a temperature between about 5 to 15°C. below the optimal growth temperature for the wild type. The restrictive conditions include a temperature between the optimal growth temperature for the organism and at least about 15° C. above the optimal growth temperature for the organism. The second permissive conditions are substantially the same as the first permissive conditions. The plants from step (a) are selected from the group consisting of monocotyledons and dicotyledons. The plants from step (a) may have been mutagenized by insertion mutagenesis with T-DNA or transposon nucleic acid sequences. The mutagen can be selected from the group consisting of nucleic acid alkylating agents, intercalating agents, ionizing radiation, heat, and sound. The alkylating and intercalating agents can be selected from the group consisting of methanesulfonate, methyl methanesulfonate, methylnitrosoguanidine, 4-nitroquinoline-1-oxide, 2-aminopurine, 5-bromouracil, ICR 191 and other acridine derivatives, ethidium bromide, nitrous acid, and N-methyl-N′-nitroso-N-nitroguanidine. The plant cells in growing step (a) are replica plated plant cells on plant leaf disks. The period of time in step (c) is equivalent to at least one growth cycle.

### EXAMPLES OF THE PREFERRED EMBODIMENTS

The following examples further illustrate the present invention. These examples are intended merely to be illustrative of the present invention and are not to be construed as being limiting.

### Example 1

Cytoplasmic Inhibition of Phytoene Desaturase in Transfected Plant Confirms that the Partial Tomato PDS Sequence Encodes Phytoene Desaturase.

Isolation of tomato mosaic virus cDNA. An 861 base pair fragment (5524-6384) from the tomato mosaic virus (fruit necrosis strain F; tom-F) containing the putative coat protein subgenomic promoter, coat protein gene, and the 3′-end was isolated by PCR using primers 5′-CTCGCAAAGTTTCGAACCAAATCCTC-3′ (upstream) (SEQ ID NO: 1) and 5′-CGGGGTACCTGGGCCCCAACCGGGGGTTCCGGGGG-3′ (downstream) (SEQ ID NO: 2) and subcloned into the HincII site of pBluescript KS−. A hybrid virus consisting of TMV-U1 and ToMV-F was constructed by swapping an 874-bp BamHI-KpnI ToMV fragment into pBGC152, creating plasmid TTO1. The inserted fragment was verified by dideoxynucleotide sequencing. A unique AvrII site was inserted downstream of the XhoI site in TTO1 by PCR mutagenesis, creating plasmid TTO1A, using the following oligonucleotides: 5′-TCCTCGAGCCTAGGCTCGCAAAGTTTCGAACCAAATCCTCA-3′ (upstream) (SEQ ID NO: 3), 5′-CGGGGTACCTGGGCCCCAACCGGGGGTTCCGGGGG-3′ (downstream) (SEQ ID NO: 4).

Isolation of a cDNA encoding tomato phytoene synthase and a partial cDNA encoding tomato phytoene desaturase. Partial cDNAs were isolated from ripening tomato fruit RNA by polymerase chain reaction (PCR) using the following oligonucleotides: PSY,

| | | |
|---|---|---|
| 5′-TATGTATGGTGCAGAAGAACAGAT-3′ | (upstream), | (SEQ ID NO: 5) |
| 5′-AGTCGACTCTTCCTCTTCTGGCATC-3′ | (downstream); | (SEQ ID NO: 6) PDS, |
| 5′-TGCTCGAGTGTGTTCTTCAGTTTTCTGTCA-3′ | (upstream), | (SEQ ID NO: 7) |
| 5′-AACTCGAGCGCTTTGATTTCTCCGAAGCTT-3′ | (downstream). | (SEQ ID NO: 8) |

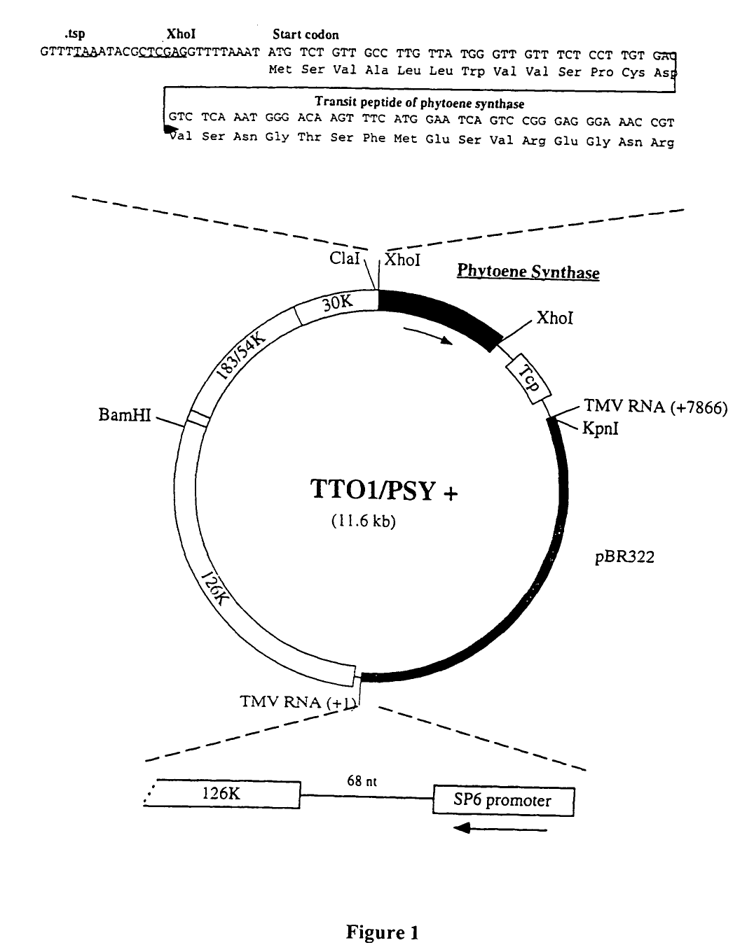

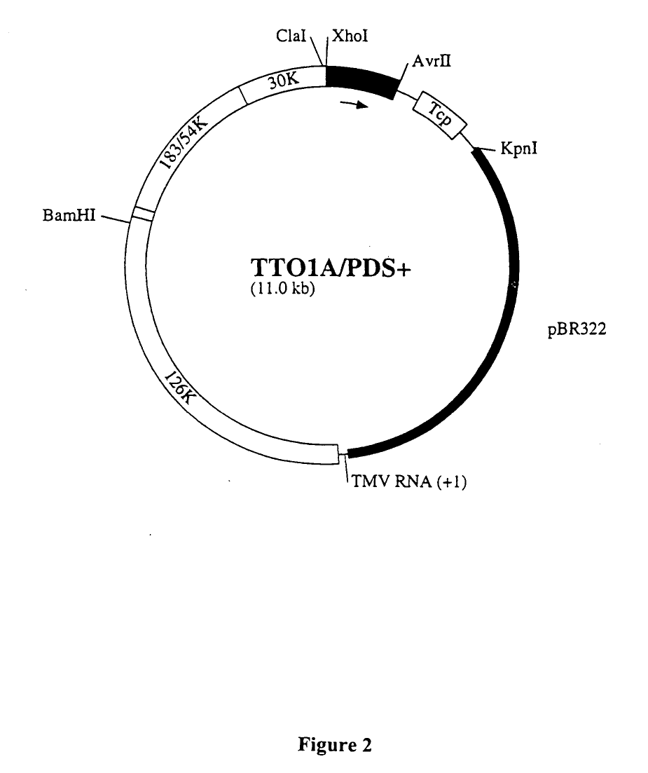

Approximately 3×10⁴ colonies from a Lycopersicon esculentum cDNA library were screened by colony hybridization using a ³²P labeled tomato phytoene synthase PCR product. Hybridization was carried out at 42° C. for 48 hours in 50% formamide, 5×SSC, 0.02 M phosphate buffer, 5× Denhart's solution, and 0.1 mg/ml sheared calf thymus DNA. Filters were washed at 65° C. in 0.1×SSC, 0.1% SDS prior to autoradiography. PCR products and the phytoene synthase cDNA clones were verified by dideoxynucleotide sequencing.
DNA sequencing and computer analysis. A PstI, BamHI fragment containing the phytoene synthase cDNA and the partial phytoene desaturase cDNA was subcloned into pBluescript® KS+ (Stratagene, La Jolla, Calif.). The nucleotide sequencing of KS+/PDS #38 and KS+/5′3′PSY was carried out by dideoxy termination using single-stranded templates (Maniatis, Molecular Cloning, 1^{st} Ed.) Nucleotide sequence analysis and amino acid sequence comparisons were performed using PCGENE® and DNA Inspector® IIE programs.
Construction of the tomato phytoene synthase expression vector. A XhoI fragment containing the tomato phytoene synthase cDNA was subcloned into TTO1. The vector TTO1/PSY+ (FIG. 1) contains the phytoene synthase cDNA in the positive orientation under the control of the TMV-U1 coat protein subgenomic promoter; while, the vector TTO1/PSY− contains the phytoene synthase cDNA in the antisense orientation.
Construction of a viral vector containing a partial tomato phytoene desaturase cDNA. A XhoI fragment containing the partial tomato phytoene desaturase cDNA was subcloned into TTO1. The vector TTO1A/PDS+ (FIG. 2) contains the phytoene desaturase cDNA in the positive orientation under the control of the TMV-U1 coat protein subgenomic promoter; while the vector TTO1A/PDS− contains the phytoene desaturase cDNA in the antisense orientation.
Transfection and analysis of N. benthamiana [TTO1/PSY+, TTO1/PSY−, TTO1Δ/PDS+, TTO1/PDS−]. Infectious RNAs from TTO1/PSY+ (FIG. 1), TTO1/PSY-TTO1/PDS+, TTO1/PDS+ were prepared by in vitro transcription using SP6 DNA-dependent RNA polymerase as described previously (Dawson et al., Proc. Natl. Acad. Sci. USA 83:1832 (1986)) and were used to mechanically inoculate N. benthamiana. The hybrid viruses spread throughout all the non-inoculated upper leaves as verified by transmission electron microscopy, local lesion infectivity assay, and polymerase chain reaction (PCR) amplification. The viral symptoms resulting from the infection consisted of distortion of systemic leaves and plant stunting with mild chlorosis. The leaves from plants transfected with TTO1/PSY+ turned orange and accumulated high levels of phytoene while those transfected with TTO1Δ/PDS+ and TTO1Δ/PDS− turned white. Agarose gel eletrophoresis of PCR cDNA isolated from virion RNA and Northern blot analysis of virion RNA indicate that the vectors are maintained in an extrachromosomal state and have not undergone any detectable intramolecular rearrangements.
Purification and analysis of carotenoids from transfected plants. The carotenoids were isolated from systemically infected tissue and analyzed by HPLC chromatography. Carotenoids were extracted in ethanol and identified by their peak retention time and absorption spectra on a 25-cm Spherisorb® ODS-1 5-m column using acetonitrile/methanol/2-propanol (85:10:5) as a developing solvent at a flow rate of 1 ml/min. They had identical retention time to a synthetic phytoene standard and β-carotene standards from carrot and tomato. The phytoene peak from N. benthamiana transfected with TTO1/PSY+ had an optical absorbance maxima at 276, 285, and 298 nm. Plants transfected with viral encoded phytoene synthase showed a ten-fold increase in phytoene compared to the levels in noninfected plants. The expression of sense and antisense RNA to a partial phytoene desaturase in transfected plants inhibited the synthesis of colored carotenoids and caused the systemically infected leaves to turn white. HPLC analysis of these plants revealed that they also accumulated phytoene. The white leaf phenotype was also observed in plants treated with the herbicide norflurazon which specifically inhibits phytoene desaturase.

This change in the levels of phytoene represents one of the largest increases of any carotenoid (secondary metabolite) in any genetically engineered plant. Plants transfected with viral-encoded phytoene synthase showed a ten-fold increase in phytoene compared to the levels in noninfected plants. In addition, the accumulation of phytoene in plants transfected with positive-sense or antisense phytoene desaturase suggests that viral vectors can be used as a potent tool to manipulate pathways in the production of secondary metabolites through cytoplasmic antisense inhibition. These data are presented by Kumagai et al., Proc. Natl. Acad. Sci. USA 92:1679-1683 (1995).

### Example 2

Expression of Bell Pepper cDNA in Transfected Plant Confirms that it Encodes Capsanthin-Capsorubin Synthase.

The biosynthesis of leaf carotenoids in Nicotiana benthamiana was altered by rerouting the pathway to the synthesis of capsanthin, a non-native chromoplast-specific xanthophyll, using an RNA viral vector. A cDNA encoding capsanthin-capsorubin synthase (Ccs), was placed under the transcriptional control of a tobamovirus subgenomic promoter. Leaves from transfected plants expressing Ccs developed an orange phenotype and accumulated high levels of capsanthin. This phenomenon was associated by thylakoid membrane distortion and reduction of grana stacking. In contrast to the situation prevailing in chromoplasts, capsanthin was not esterified and its increased level was balanced by a concomitant decrease of the major leaf xanthophylls, suggesting an autoregulatory control of chloroplast carotenoid composition. Capsanthin was exclusively recruited into the trimeric and monomeric light-harvesting complexes of Photosystem II. This demonstration that higher plant antenna complexes can accommodate non-native carotenoids provides compelling evidence for functional remodeling of photosynthetic membranes by rational design of carotenoids.

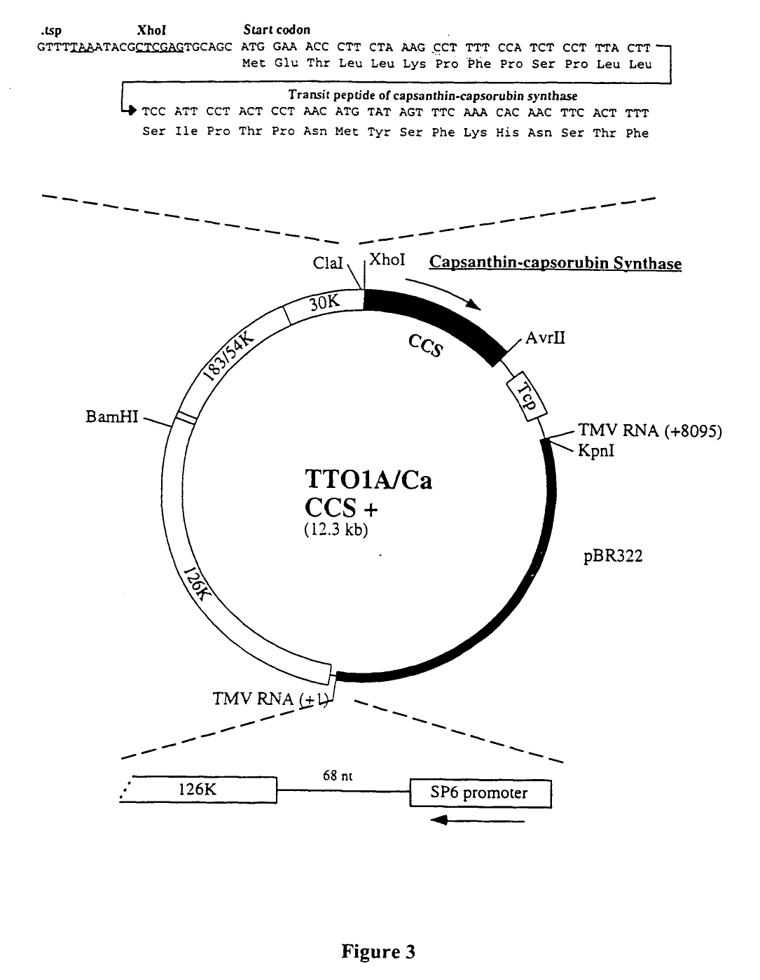

Construction of the Ccs expression vector. Unique XhoI, AvrII sites were inserted into the bell pepper capsanthin-capsorubin synthase (Ccs) cDNA by polymerase chain reaction (PCR) mutagenesis using oligonucleotides: 5′-GCCTCGAGTGCAGCATGGAAACCCTTCTAAAGCTTTTCC-3′ (upstream) (SEQ ID NO: 9), 5′-TCCCTAGGTCAAAGGCTCTCTATTGCTAGATTGCCC-3′ (downstream) (SEQ ID NO: 10). The 1.6-kb XhoI, AvrII cDNA fragment was placed under the control of the TMV-U1 coat protein subgenomic promoter by subcloning into TTO1A, creating plasmid TTO1A CCS+ (FIG. 3) in the sense orientation as represented by FIG. 3.

Carotenoid analysis. Twelve days after inoculation upper leaves from 12 plants were harvested and lyophilized. The resulting non-saponified extract was evaporated to dryness under argon and weighed to determine the total lipid content. Pigment analysis from the total lipid content was performed by HPLC and also separated by thin layer chromatography on silica gel G using hexane/acetone (60v/40v). Plants transfected with TTO1A CCS+ accumulated high levels of capsanthin (36% of total carotenoids).

### Example 3

Expression of Bacterial CrtB Gene in Transfected Plants Confirms that it Encodes Phytoene Synthase.

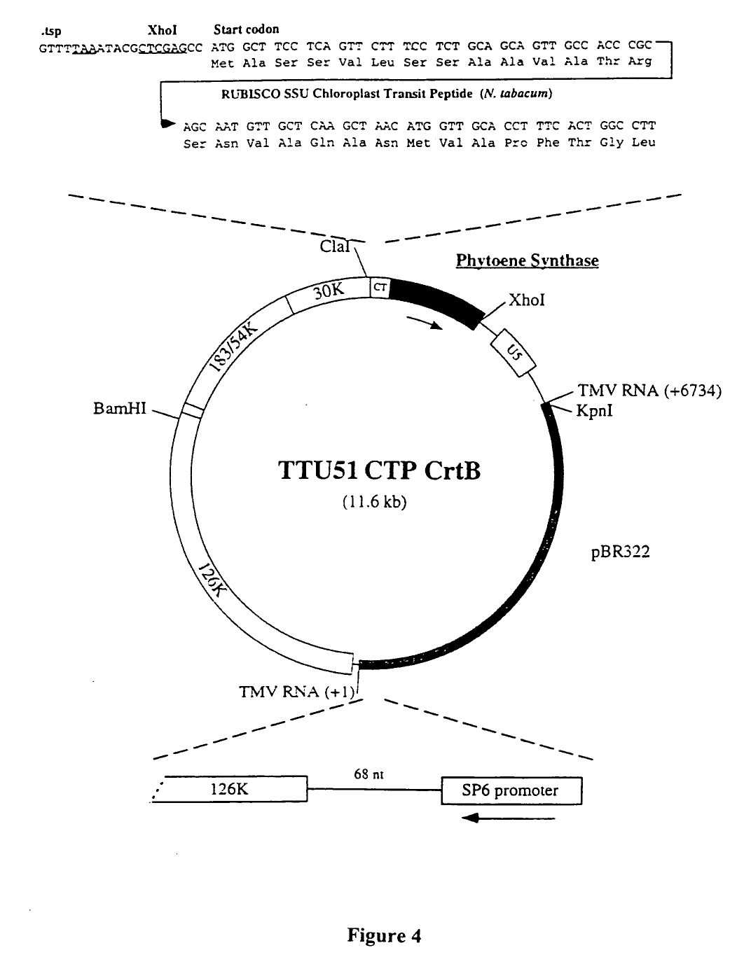

We developed a new viral vector, TTU51, consisting of tobacco mosaic virus strain U1 (TMV-U1) (Goelet et al., Proc. Natl. Acad. Sci. USA 79:5818-5822 (1982)), and tobacco mild green mosaic virus (TMGMV; U5 strain) (Solis et al., “The complete nucleotide sequence of the genomic RNA of the tobamovirus tobacco mild green mosaic virus” (1990)). The open reading frame (ORF) for Erwinia herbicola phytoene synthase (CrtB) (Armstrong et al., Proc. Natl. Acad. Sci. USA 87:9975-9979 (1990)) was placed under the control of the tobacco mosaic virus (TMV) coat protein subgenomic promoter in the vector TTU51. This construct also contained the gene encoding the chloroplast targeting peptide (CTP) for the small subunit of ribulose-1,5-bisphosphate carboxylase (RUBISCO) (O'Neal et al., Nucl. Acids Res. 15:8661-8677 (1987)) and was called TTU51 CTP CrtB as represented by FIG. 4. Infectious RNA was prepared by in vitro transcription using SP6 DNA-dependent RNA polymerase (Dawson et al, Proc. Natl. Acad. Sci. USA 83:1832-1836 (1986)); Susek et al., Cell 74:787-799 (1993)) and was used to mechanically inoculate N. benthamiana. The hybrid virus spread throughout all the non-inoculated upper leaves and was verified by local lesion infectivity assay and polymerase chain reaction (PCR) amplification. The leaves from plants transfected with TTU51 CTP CrtB developed an orange pigmentation that spread systemically during plant growth and viral replication.

Leaves from plants transfected with TTU51 CTP CrtB had a decrease in chlorophyll content (result not shown) that exceeded the slight reduction that is usually observed during viral infection. Since previous studies have indicated that the pathways of carotenoid and chlorophyll biosynthesis are interconnected (Susek et al., Cell 74:787-799 (1993)), we decided to compare the rate of synthesis of phytoene to chlorophyll. Two weeks post-inoculation, chloroplasts from plants infected with TTU51 CTP CrtB transcripts were isolated and assayed for enzyme activity. The ratio of phytoene synthetase to chlorophyll syntheses was 0.55 in transfected plants and 0.033 in uninoculated plants (control). Phytoene synthase activity from plants transfected with TTU51 CTP CrtB was assayed using isolated chloroplasts and labeled [¹⁴C] geranylgeranyl PP. There was a large increase in phytoene and an unidentified C₄₀ alcohol in the CrtB plants.

Phytoene Synthetase Assay.

The chloroplasts were prepared as described previously (Camara, Methods Enzymol. 214:352-365 (1993)). The phytoene synthase assays were carried out in an incubation mixture (0.5 ml final volume) buffered with Tris-HCL, pH 7.6, containing [¹⁴C] geranylgeranyl PP (100,000 cpm) (prepared using pepper GGPP synthase expressed in E. coli), 1 mM ATP, 5 mM MnCl₂, 1 mM MgCl₂, Triton X-100 (20 mg per mg of chloroplast protein) and chloroplast suspension equivalent to 2 mg protein. After 2 h incubation at 30° C., the reaction products were extracted with chloroform methanol (Camara, supra) and subjected to TLC onto silicagel plate developed with benzene/ethyl acetate (90/10) followed by autoradiography.

Chlorophyll Synthetase Assay.

For the chlorophyll synthetase assay, the isolated chloroplasts were lysed by osmotic shock before incubation. The reaction mixture (0.2 ml, final volume) consisting of 50 mM Tris-HCL (pH 7.6) containing [¹⁴C] geranylgeranyl PP (100,000 cpm), 5 MgCl₂, 1 mM ATP, and ruptured plasmid suspension equivalent to 1 mg protein was incubated for 1 hr at 30° C. The reaction products were analyzed as described previously.

Plasmid Constructions.

The chloroplast targeting, phytoene synthase expression vector, TTU51 CTP CrtB as represented in FIG. 4, was constructed in several subcloning steps. First, a unique SphI site was inserted in the start codon for the Erwinia herbicola phytoene synthase gene by polymerase chain reaction (PCR) mutagenesis (Saiki et al., Science 230:1350-1354 (1985)) using oligonucleotides CrtB M1S 5′-CCA AGC TTC TCG AGT GCA GCA TGC AGC AAC CGC CGC TGC TTG AC-3′ (upstream) (SEQ ID NO: 11) and CrtB P300 5′-AAG ATC TCT CGA GCT AAA CGG GAC GCT GCC AAA GAC CGG CCG G-3′ (downstream) (SEQ ID NO: 12). The CrtB PCR fragment was subcloned into pBluescript® (Stratagene) at the EcoRV site, creating plasmid pBS664. A 938 bp SphI, XhoI CrtB fragment from pBS664 was then subcloned into a vector containing the sequence encoding the N. tabacum chloroplast targeting peptide (CTP) for the small subunit of RUBISCO, creating plasmid pBS670. Next, the tobamoviral vector, TTU51, was constructed. A 1020 base pair fragment from the tobacco mild green mosaic virus (TMGMV; U5 strain) containing the viral subgenomic promoter, coat protein gene, and the 3′-end was isolated by PCR using TMGMV primers 5′-GGC TGT GAA ACT CGA AAA GGT TCC GG-3′ (upstream) (SEQ ID NO: 13) and 5-CGG GGT ACC TGG GCC GCT ACC GGC GGT TAG GGG AGG-3′ (downstream) (SEQ ID NO: 14), subcloned into the HincII site of Bluescript KS-, and verified by dideoxynucleotide sequencing. This clone contains a naturally occurring duplication of 147 base pairs that includes the whole upstream pseudoknot domain in the 3′ noncoding region. The hybrid viral cDNA consisting of TMV-U1 and TMGMV was constructed by swapping a 1-Kb XhoI-KpnI TMGMV fragment into TTO1 (Kumagai et al., Proc. Natl. Acad. Sci. USA 92:1679-1683 (1995)), creating plasmid TTU51. Finally, the 1.1 Kb XhoI CTP CrtB fragment from pBS670 was subcloned into the XhoI of TTU51, creating plasmid TTU51 CTP CrtB. As a CTP negative control, a 942 bp XhoI fragment containing the CrtB gene from pBS664 was subcloned into TTU51, creating plasmid TTU51 CrtB #15.

### Example 4

Expression of Bacterial Phytoene Desaturase (Crtf) Gene in Transfected Plants Confers Resistance to Norflurazon Herbicide.

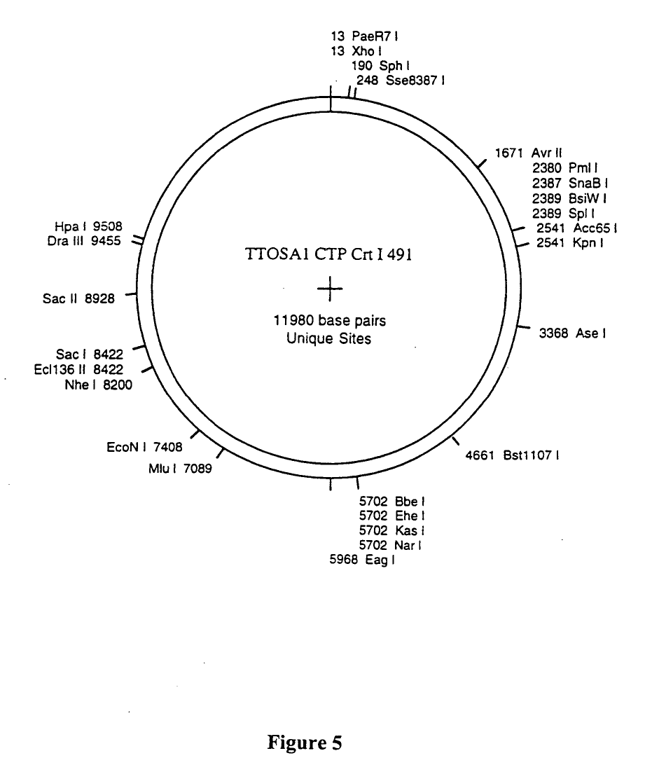

Erwinia phytoene desaturase (PDS), which is encoded by the gene CrtI (Armstrong et al., 1990), converts phytoene to lycopene through four desaturation steps. While plant PDS is sensitive to the bleaching herbicide norflurazon, Erwinia PDS is not inhibited by norflurazon (Misawa et al., Plant J. 6(4):481-489 (1994)). The open reading frame (ORF) for CrtI was placed under the control of the tobacco mosaic virus (TMV) coat protein subgenomic promoter in the vector TTOSA1. This construct also contained the gene encoding the chloroplast targeting peptide (CTP) for the small subunit of ribulose-1,5-bisphosphate carboxylase (RUBISCO) and was called TTOSA1 CTP CrtI 491 #7 Infectious RNA was prepared by in vitro transcription using SP6 DNA-dependent RNA polymerase (Dawson et al., Proc. Natl. Acad. Sci. USA 83:1832-1836 (1986)) and was used to mechanically inoculate N. benthamiana. The hybrid virus spread throughout all the non-inoculated upper leaves, conferring resistance to norflurazon to the entire plant. TTOSA1 CTP CrtI 491 #7 (FIG. 5) inoculated plants remained green instead of bleaching white, and maintained higher levels of β-carotene compared to uninoculated control plants.

Plasmid Constructions.

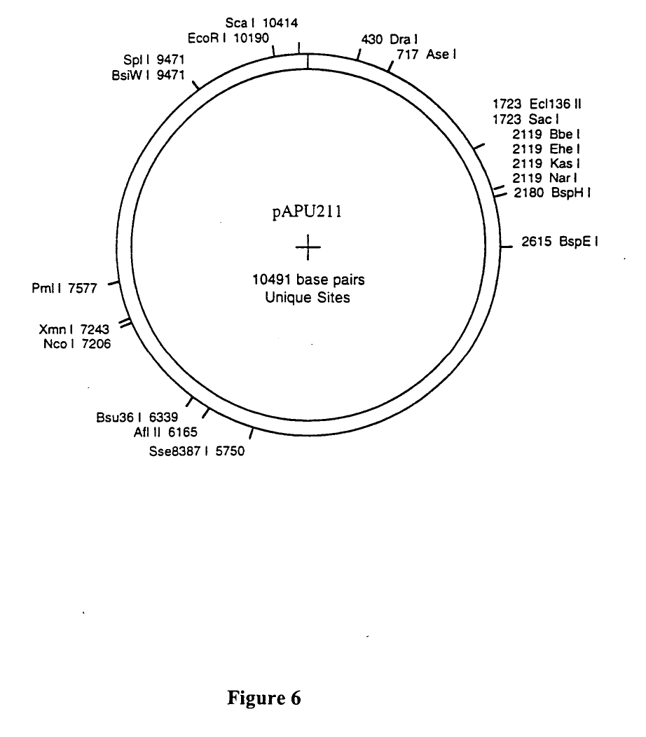

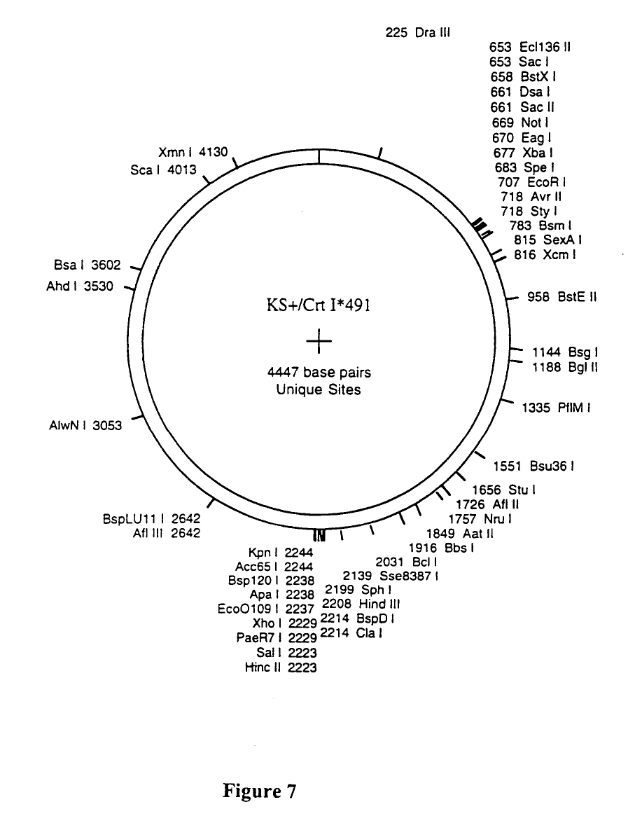

The chloroplast targeting, bacterial phytoene desaturase expression vector, TTOSA1 CTP CrtI 491 #7 (FIG. 5) was constructed as follows. First, a unique SphI site was inserted in the start codon for the Erwinia herbicola phytoene desaturase gene (plasmid pAU211, (FIG. 6) by polymerase chain reaction (PCR) mutagenesis using the oligonucleotides CrtI HSM1 5′-GA CAG AAG CTT TGC AGC ATG CAA AAA ACC GTT-3′ (upstream) (SEQ ID NO: 16) and IQ419A 5′-CGC GGT CAT TGC AGA TCC TCA ATC ATC AGG C-3′ (downstream) (SEQ ID NO: 17). The 1504 bp CrtI PCR fragment was subcloned into pBluescript® (Stratagene) by inserting it between the EcoRV and HindIII sites, creating plasmid KS+/CrtI* 491 (FIG. 7). A 1481 bp SphI, AvrII CrtI fragment from plasmid KS+/CrtI* 491 was then subcloned into the tobamoviral vector TTOSA1, creating TTOSA1 CTP CrtI 491 #7.

Treatment of Transfected Plants with Norflurazon and Results.

Starting 7 days after viral inoculation, the plants were treated with 5 ml of a 10 mg/ml Solicam®DF (Sandoz Agro, Inc.) norflurazon herbicide solution [(4-chloro-5-(methylamino)-2-(alpha, alpha, alpha-trifluoro-m-tolyl)-3(2H)-pyridazinone)] every 4 days by applying to leaves and soil. Five days after initiating treatment, uninfected plants were almost entirely white, especially in the upper leaves and meristematic areas. Plants infected with TTOSA1 CTP CrtI 491 #7 were still green and were almost identical in appearance to the non-norflurazon treated infected controls.

Leaf Analysis.

The spread of the virally expressed CrtI gene throughout the plant was verified by Northern blotting (Alwine et al., Proc. Natl. Acad. Sci. USA 74:5350-5354 (1977)). Viral RNA was purified from uninoculated upper leaves and was probed with the 1.5 kb CrtI gene. Positive results were obtained from plants inoculated with TTOSA1 CTP CrtI 491 #7.

Leaf tissue from a TTOSA1 CTP CrtI 491 #7 infected plant was examined for β-carotene levels. Treating an uninoculated control plant with norflurazon resulted in severely depressed β-carotene levels (7.8% of the wild-type level). However, when a plant which had been previously inoculated with the viral construct TTOSA1 CTP CrtI 491 #7 was treated with norflurazon, the β-carotene level were partially restored (28.3% of the wild-type level). This is similar to the level of β-carotene in TTOSA1 CTP CrtI 491 #7 samples not treated with norflurazon (an average of 38.3% of wild-type), indicating that the herbicide norflurazon had little effect on β-carotene levels in previously transfected plants. The expression of the bacterial phytoene desaturase in systematically infected tissue conferred resistance to the herbicide norflurazon.

### Example 5

Expression of 5-enolpyruvylshikimate-3-phosphate Synthase (EPSPS) Genes in Plants Confers Resistance to Roundup® Herbicide.

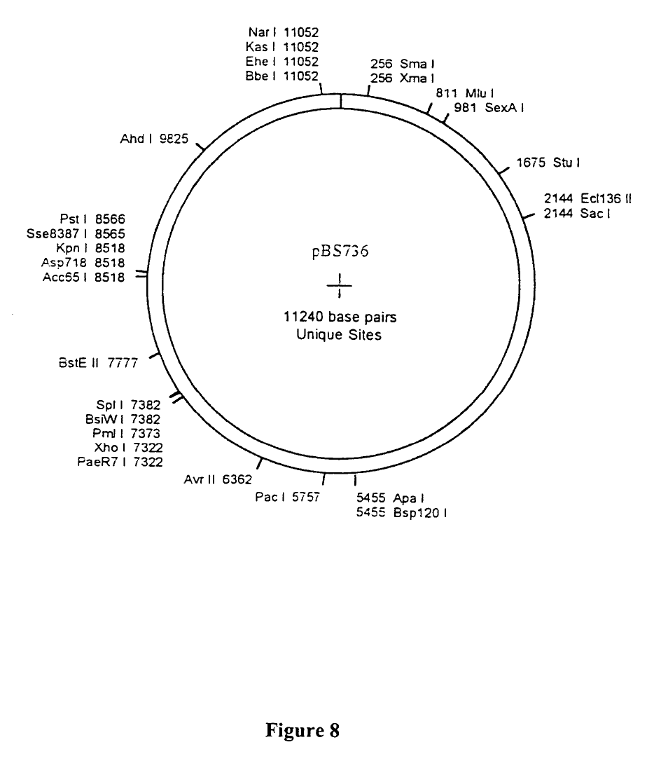

Systemic expression via a recombinant viral vector of 5-enolpyruvylshikimate-3-phosphate synthase (EPSPS) genes in plants confers resistance to Roundup® herbicide. See also della-Cioppa, et al., “Genetic Engineering of herbicide resistance in plants,” Frontiers of Chemistry: Biotechnology, Chemical Abstract Service, ACS, Columbus, Ohio, pp. 665-70 (1989). The purpose of this experiment is to provide a method to systemically express EPSPS genes via a recombinant viral vector in fully-grown plants. Transfected plants that overproduce the enzyme EPSPS in vegetative tissue (root, stem, and leaf) are resistant to Roundup® herbicide. The present invention provides a method for the production of plasmid-targeted EPSPS in plants via an RNA viral vector. A dual subgenomic promoter vector encoding the full-length EPSPS gene from Nicotiana tabacum (Class I EPSPS) is shown in plasmid pBS736. Systemic expression of the Nicotiana tabacum Class I EPSPS confers resistance to Roundup® herbicide in whole plants and tissue culture. FIG. 8 shows plasmid pBS736.

### Example 6

Cytoplasmic Inhibition of 5-enolpyruvylshikimate-3-phosphate Synthase (EPSPS) Genes in Plants Blocks Aromatic Amino Acid Biosynthesis.

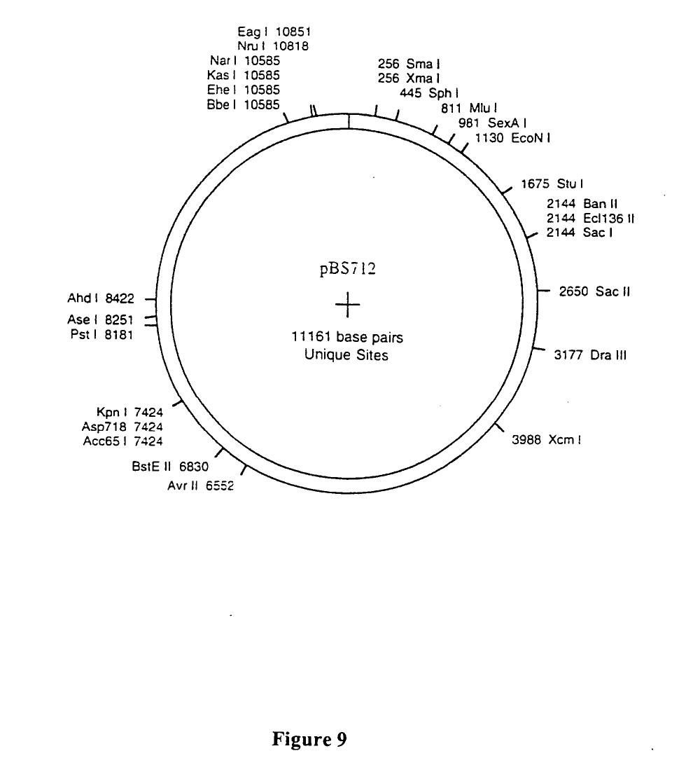

Cytoplasmic inhibition of 5-enolpyruvylshikimate-3-phosphate synthase (EPSPS) genes in plants blocks aromatic amino acid biosynthesis and causes a systemic bleaching phenotype similar to Roundup® herbicide. See also della-Cioppa, et al., “Genetic Engineering of herbicide resistance in plants,” Frontiers of Chemistry: Biotechnology, Chemical Abstract Service, ACS, Columbus, Ohio, pp. 665-70 (1989). A dual subgenomic promoter vector encoding 1097 base pairs of an antisense EPSPS gene from Nicotianan tabacum (Class I EPSPS) is shown in plasmid pBS712. FIG. 9 shows plasmid pBS712. Systemic expression of the Nicotiana tabacum Class I EPSPS gene in the antisense orientation causes a systemic bleaching phenotype similar to Roundup® herbicide.

### Example 7

Exemplary Complementation Analysis.

A transgenic plant or naturally occurring plant mutant may have a non-functional gene such as the one which produces EPSP synthase. A plant deficient or lacking in the EPSP synthase gene could grow only in the presence of added aromatic amino acids. Transfection of plants with a viral vector containing a functional EPSP synthase gene or cDNA sequence encoding the same would cause the plant to produce a finctional EPSP synthase gene product. A plant so transfected would then be able to grow normally without added aromatic amino acids to its environment. In this transfected plant, the EPSP synthase mutation in the plant would be complemented in trans by the viral nucleic acid sequence containing the native or foreign EPSP synthase cDNA sequence.

### Example 8

Expression of Methylotrophic Yeast ZZA1 Gene in Transfected Plants Confirms that it Encodes Alcohol Oxidase.

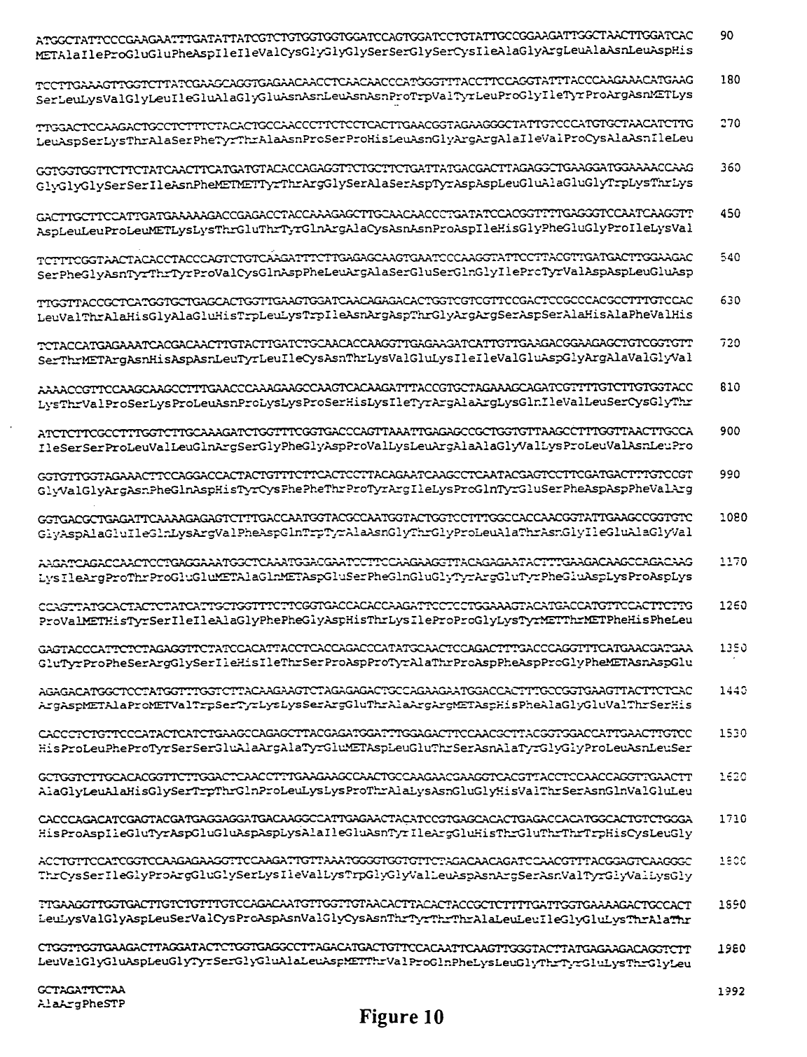

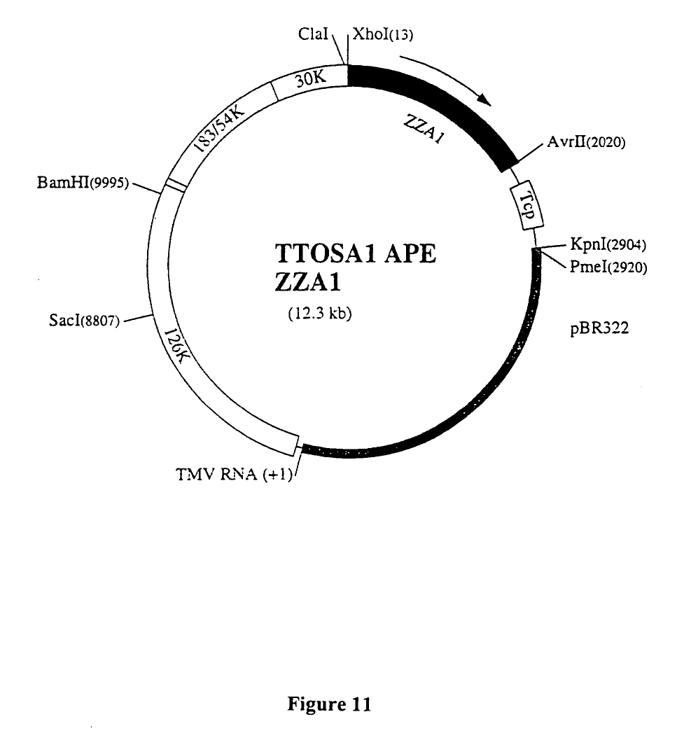

A genomic clone encoding alcohol oxidase ZZA1, the first enzyme involved in methanol utilization, was isolated from a newly described Pichia pastoris strain. Kumagai et al., Bio/Technology 11:606-610 (1993). Sequence analysis indicates that gene encodes a polypepide of approximately 72-kDa (FIG. 10). Comparison of the amino acid sequence to Pichia pastoris AOX1 and AOX2 alcohol oxidases indicates that they show 97.4% and 96.4% similarity to each other, respectively. The open reading frame (ORF) for alcohol oxidase, from the a genomic clone containing ZZA1, was placed under the control of the tobamoviral subgenomic promoter in TTO1A, a hybrid tobacco mosaic virus (TMV) and tomato mosaic virus (ToMV) vector. Infectious RNA from TTO1APE ZZA1 (FIG. 11) was prepared by in vitro transcription using SP6 DNA-dependent RNA polymerase and used to mechanically inoculate N. benthamiana. The 72-kDa protein accumulated in systemically infected tissue and was analyzed by immunoblotting, using Pichia pastoris alcohol oxidase as a standard. No detectable cross-reacting protein was observed in the noninfected N. benthamiana control plant extracts.

Isolation of the Alcohol Oxidase Gene.

Three hundred nanograms of the yeast Pichia pastoris genomic DNA digested with PstI and XhoI was amplified by PCR using a 25-mer oligonucleotide (5′-TTG CAC TCT GTT GGC TCA TGA CGA T-3′) (SEQ ID NO: 17) corresponding to the nucleotide sequence of AOX1 promoter and a 26-mer oligonucleotide (5′-CAA GCT TGC ACA AAC GAA CGT CTC AC-3′) (SEQ ID NO: 18) corresponding to a nucleotide sequence derived from the AOX1 terminator. The PCR conditions using Thermus aquaticus DNA polymerase (2.5U; Perkin-Elmer Cetus) consisted of an initial 2 minute incubation at 97° C. followed by two cycles at 97° C. (1 min.), 45° C. (1 min.), 60° C. (1 min.), thirty-five cycles at 94° C. (1 min.), 45° C. (1 min.), 60° C. (1 min.), and a final DNA polymerase extension at 60° C. for 7 min. The 3273 base pair fragment containing ZZA1 gene was phenol/chloroform treated and precipitated with ammonium acetate/ethanol. After digestion with SacI the fragment was purified by 1% low melt agarose electrophoresis and subcloned into the SacI/EcoRV sites in pBluescript KS−. The alcohol oxidase genomic clone KS-AO7′8′ was characterized by restriction mapping and dideoxynucleotide sequencing.

Plasmid Constructions.

Unique XhoI, AvrII sites were inserted into the Pichia pastoris clone KS-AO7′8′ by polymerase chain reaction (PCR) mutagenesis using oligonucleotides: 5′-CAC TCG AGA GCA TGG CTA TTC CCG AAG AAT TTG ATA TTA TCG-3′ (upstream) (SEQ ID NO: 19) and 5′-TCC CTA GGT TAG AAT CTA GCA AGA CCG GTC TTC TCG-3′ (downstream) (SEQ ID NO: 20). The 2.0-kb XhoI, AvrII ZZA1 PCR fragment was subcloned into pTTO1APE, creating plasmid TTO1APE ZZA1.

### Example 9

Rapid, High-level Expression of Rice OS103 cDNA in Transfected Plants Confirms that it Encodes Glycosylated rice α-amylase.

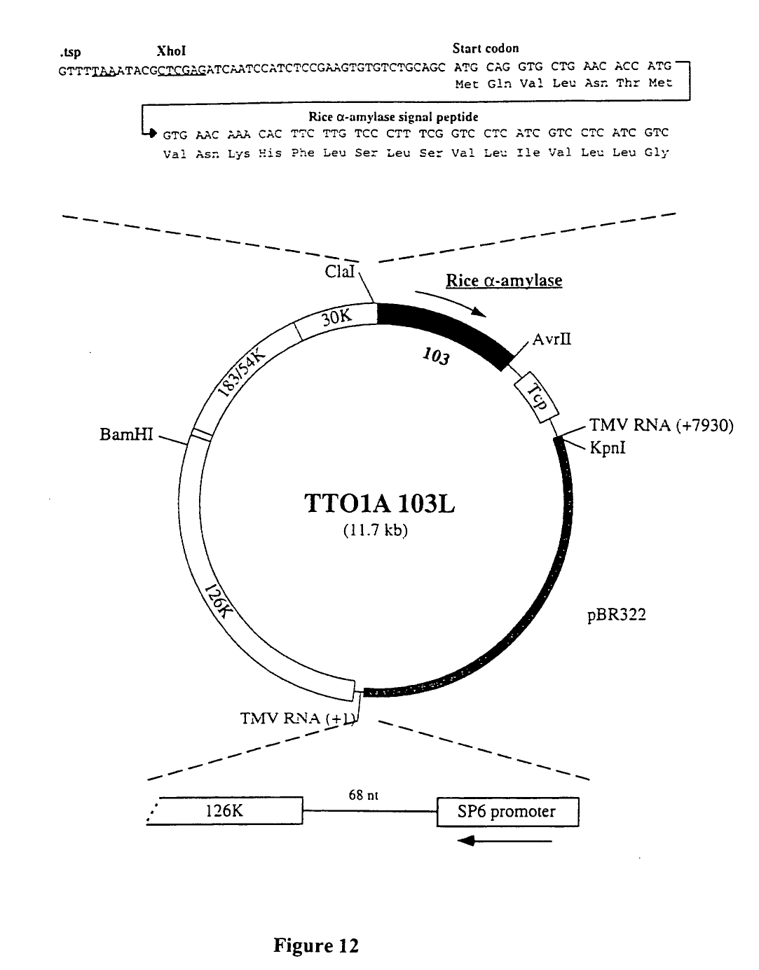

The open reading frame (ORF) for rice α-amylase, from the cDNA clone pOS103 (O'Neill et al., Mol. Gen. Genet. 221:235-244 (1990)), was placed under the control of the tobamoviral subgenomic promoter in TTO1A (Kumagai et al., Proc. Natl. Acad. Sci. USA 92:1679-1683 (1995)), a hybrid tobacco mosaic virus (TMV) and tomato mosaic virus (ToMV) vector. Infectious RNA from TTO1A 103L (FIG. 12) was prepared by in vitro transcription using SP6 DNA-dependent RNA polymerase and used to mechanically inoculate N. benthamiana. The hybrid virus spread throughout the noninoculated upper leaves as verified by transmission electron microscopy, local lesion infectivity assay, and PCR amplification. The viral symptoms consisted of plant stunting with mild chlorosis and distortion of systemic leaves. The 46-kDa α-amylase accumulated to levels of at least 5% total soluble protein, and was analyzed by immunoblotting, using yeast expressed α-amylase as a standard. No detectable cross-reacting protein was observed in the noninfected N. benthamiana control plant extracts. The expression level of the recombinant enzyme produced in transfected plants was at least ten times higher than the amount of thermostable bacterial α-amylase produced in transgenic tobacco. The α-amylase was purified using ion exchange chromatography and its structural and biological properties were analyzed. The secreted protein had an approximate relative molecular mass of 46 kDa, cross-reacted with anti-α-amylase antibody, and hydrolyzed starch and oligomaltose in an in vitro assay.

The recombinant enzyme from transfected N. benthamiana was glycosylated at an asparagine residue via an N-glycosidic linkage. The heterologously expressed α-amylases from transfected N. benthamiana and from transformed strains of S. cerevisiae and P. pastoris were treated with endo-H and were compared by Western blot/SDS-PAGE analysis. There was an equivalent mobility shift for the enzymes expressed in S. cerevisiae and P. pastoris. The extent of the change in mobility suggests that the yeast expressed enzymes are hyperglycosylated while the recombinant protein from transfected plants is similar to that of the native rice α-amylase. While it is known that mannose-rich and complex oligosaccharide side chains are covalently attached to the mature rice seed α-amylase (Mitsui et al., Plant Physiol. 82:880-884 (1986)), the actual carbohydrate composition and structure of the recombinant plant glycoprotein remains to be determined.

MALDI-TOF analysis revealed that the relative molecular mass (Mᵣ) of the N. benthamiana expressed sample was 46,064 Da. The Mᵣ of the α-amylase determined by MALDI-TOF was 918 Da larger than the Mᵣ derived from the amino acid sequence (PCGENE). The change in molecular mass (ΔMᵣ) of the plant expressed enzyme was smaller than the ΔMᵣ of α-amylases produced in yeast. This result suggests that there is a difference in glycosylation patterns between foreign proteins expressed in plants and those that are secreted in yeast.

Plasmid Constructions.

Unique XhoI, AvrII sites were inserted into the rice α-amylase pOS103 cDNA by PCR mutagenesis using oligonucleotides: 5′-CTC TCG AGA TCA ATC ATC CAT CTC CGA AGT GTG TCT GC-3′ (upstream) (SEQ ID NO: 21) and 5′-TCC CTA GGT CAG ATT TTC TCC CAG ATT GCG TAG C-3′ (downstream) (SEQ ID NO: 22). The 1.4-kb XhoI, AvrII OS 103 PCR fragment was subcloned into pTTO1A, creating plasmid TTO1A 103L.

Purification, Imunological Detection, and In Vitro Assay of α-amylase.

Ten days after inoculation, total soluble protein was isolated from 10 g of upper, noninoculated N. benthamiana leaf tissue. The leaves were frozen in liquid nitrogen and ground in 20 ml of 5% 2-mercaptoethanol/10 mM Tris-bis propane, pH 6.0. The suspension was centrifuged and the supernatant, containing recombinant α-amylase, was bound to a POROS® 50 HQ ion exchange column (PerSeptive Biosystems). The α-amylase was eluted with a linear gradient of 0.0-1 M NaCl in 50 mM Tris-bis propane pH 7.0. The α-amylase eluted in fraction 16, 17 and its enzyme activity was analyzed (Sigma Kit #576-3). Fractions containing cross-reacting material to α-amylase antibody were concentrated with a Centriprep-30® (Amicon) and the buffer was exchanged by diafiltration (50 mM Tris-bis propane, pH 7.0). The sample was then loaded on a POROS HQ/M column (Perceptive Biosystems), eluted with a linear gradient of 0.0-1 M NaCl in 50 mM Tris-bis propane pH 7.0, and assayed for α-amylase activity. Fractions containing cross-reacting material to α-amylase antibody were concentrated with a Centriprep-30 and the buffer was exchanged by diafiltration (20 mM Sodium Acetate/HEPES/MES, pH 6.0). The sample was finally loaded on a POROS HS/M column (Perceptive Biosystems), eluted with a linear gradient of 0.0-1 M NaCl in 20 mM Sodium Acetate/HEPES/MES, pH 6.0, and assayed for α-amylase activity. Total soluble plant protein concentrations were determined using bovine serum albumin as a standard. The proteins were analyzed on a 0.1% SDS/10% polyacrylamide gel and transferred by electroblotting for 1 hour to a nitrocellulose membrane. The blotted membrane was incubated for 1 hr with a 2000-fold dilution of anti-α-amylase antiserum. Using standard protocols, the antisera was raised in rabbits against S. cerevisiae expressed rice α-amylase. The enhanced chemiluminescence horseradish peroxidase-linked, goat anti-rabbit IgG assay (Cappel Laboratories) was performed according to the manufacturer's (Amersham) specifications. The blotted membrane was subjected to film exposure times of up to 10 sec. The quantity of total recombinant α-amylase in an extracted leaf sample was determined (using a 1-sec exposure of the blotted membrane) by comparing the crude extract chemiluminescent signal to the signal obtained from known quantities of α-amylase. Shorter and longer chemiluminescent exposure times of the blotted membrane gave the same quantitative results.

Analysis of Post-Translational Modifications of Recombinant α-amylases.

Approximately 5 μg of recombinant protein was dissolved in 1 M acetic acid and subjected to matrix-assisted laser desorption/ionization time of flight (MALDI-TOF) analysis (Karas et al., Anal. Chem. 60:2299-2301 (1988)). For treatment with endo-B-N-acetylglucosaminidase H (endo H), 2 μg of the recombinant α-amylases were denatured in 0.5% SDS/1% β-mercaptoethanol at 100° C. for 10 minutes. After the addition of 500 U of endo H (New England Biolabs) the samples were incubated at 37° C. for 4 hours in 50 mM sodium citrate (pH 5.5 @25° C.) and then subjected to Western blot analysis using anti-α-amylase antiserum.

### Example 10

Expression of Chinese Cucumber cDNA Clone pQ21D in Transfected Plants Confirms that it Encodes α-Trichosanthin.

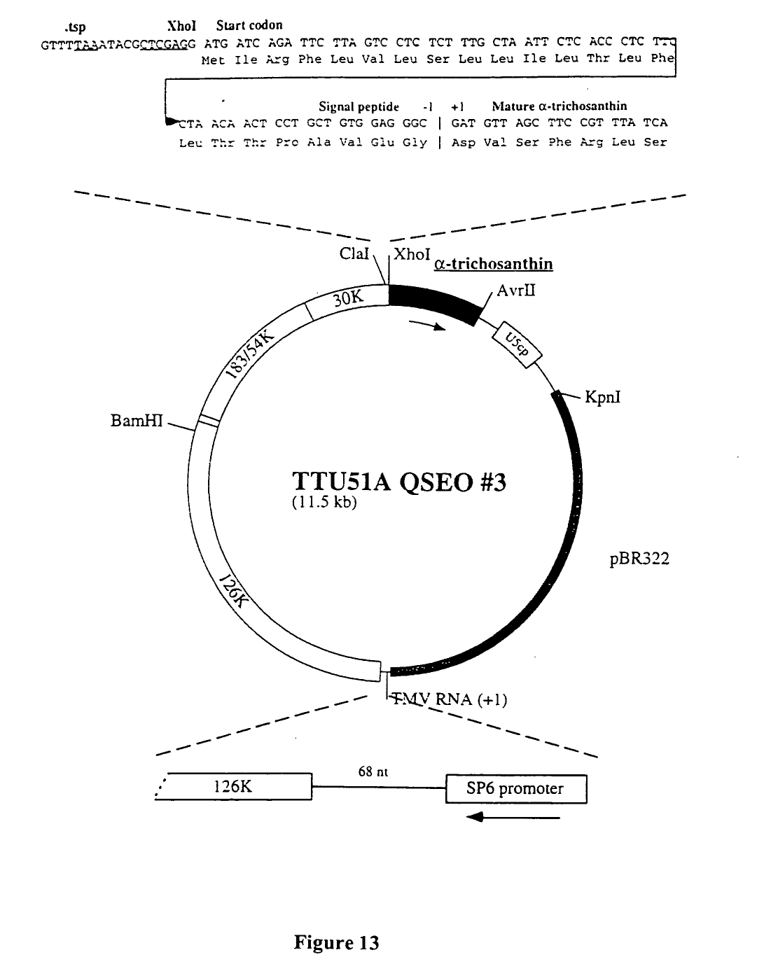

We have developed a plant viral vector that directs the expression of α-trichosanthin in transfected plants. The open reading frame (ORF) for α-trichosanthin, from the genomic clone SEO, was placed under the control of the TMV coat protein subgenomic promoter. Infectious RNA from TTU51A QSEO #3 (FIG. 13) was prepared by in vitro transcription using SP6 DNA-dependent RNA polymerase and was used to mechanically inoculate N. benthamiana. The hybrid virus spread throughout all the non-inoculated upper leaves as verified by local lesion infectivity assay, and PCR amplification. The viral symptoms consisted of plant stunting with mild chlorosis and distortion of systemic leaves. The 27-kDa α-trichosanthin accumulated in upper leaves (14 days after inoculation) and cross-reacted with an anti-trichosanthin antibody.

Plasmid Constructions.

An 0.88-kb XhoI, AvrII fragment, containing the α-trichosanthin coding sequence, was anplified from genomic DNA isolated from Trichosanthes kirilowii Maximowicz by PCR mutagenesis using oligonucleotides QMIX: 5′-GCC TCG AGT GCA GCA TGA TCA GAT TCT TAG TCC TCT CTT TGC-3′ (upstream) (SEQ ID NO: 23) and Q1266A 5′-TCC CTA GGC TAA ATA GCA TAA CTT CCA CAT CA AAGC-3′ (downstream) (SEQ ID NO: 24). The α-trichosanthin open reading frame was verified by dideoxy sequencing, and placed under the control of the TMV-U1 coat protein subgenomic promoter by subcloning into TTU51A, creating plasmid TTU51A QSEO #3.

In Vitro Transcriptions, Inoculations, and Analysis of Transfected Plants.

N. benthaminana plants were inoculated with in vitro transcripts of Kpn I-digested TTU51A QSEO #3 as previously described (Dawson et al., supra). Virions were isolated from N. benthamiana leaves infected with TTU51A QSEO #3 transcripts.

Purification, Immunological Detection, and In Vitro Assay of α-Trichosanthin.

Two weeks after inoculation, total soluble protein was isolated from upper, noninoculated N. benthamiana leaf tissue and assayed from cross-reactivity to a α-trichosanthin antibody. The proteins from systemically infected tissue were analyzed on a 0.1% SDS/12.5% polyacrylamide gel and transferred by electroblotting for 1 hr to a nitrocellulose membrane. The blotted membrane was incubated for 1 hr with a 2000-fold dilution of goat anti-α-trichosanthin antiserum. The enhanced chemiluminescence horseradish peroxidase-linked, rabbit anti-goat IgG assay (Cappel Laboratories) was performed according to the manufacturer's (Amersham) specifications. The blotted membrane was subjected to film exposure times of up to 10 sec. Shorter and longer chemiluminescent exposure times of the blotted membrane gave the same quantitative results.

### Example 11

Expression of Human β-Globin cDNA Clone in Transfected Plants Confirms that it Encodes Hemoglobin.

The hemoglobin expression vector, RED1, was constructed in several subcloning steps. A unique SphI site was inserted in the start codon for the human β-globin and an XbaI site was placed downstream of the stop codon by PCR mutagenesis by using oligonucleotides 5′-CAC TCG AGA GCA TGC TGC ACC TGA CTC CTG AGG AGA AG-3′ (upstream) (SEQ ID NO: 25) and 5′-CGT CTA GAT TAG TGA TAC TTG TGG GCC AGC GCA TTA GC-3′ (downstream) (SEQ ID NO: 26). The 452 bp SphI-XbaI hemoglobin fragment was subcloned into the SphI-AvrII site of a modified tobamoviral vector. This construct consists of a 1020 bp fragment from the tobacco mild green mosaic virus (TMGMV; U5 strain) containing the viral subgenomic promoter, coat protein gene, and the 3′-end that was isolated by PCR using TMGMV primers 5′-GGC TGT GAA ACT CGA AAA GGT TCC GG-3′ (upstream) (SEQ ID NO: 27) and 5′-CGG GGT ACC TGG GCC GCT ACC GGC GGT TAG GGG AGG-3′ (downstream) (SEQ ID NO: 28). In this vector, an artificial 40 base pair 5′ untranslated coat protein leader was fused to a hybrid cDNA encoding rice α-amylase signal peptide and human β-globin.

A hybrid sequence encoding rice alpha-amylase signal peptide and β-chain of human hemoglobin was placed under the control of the tobacco mosaic virus (TMV-U1) coat protein subgenomic promoter. Infectious RNA was made in vitro and directly applied to N. benthamiana. One to two weeks post-inoculation transfected plants had accumulated recombinant hemoglobin. The 16-KDa β-globin accumulated in systemically infected leaves and was analyzed by immunoblotting, using human hemoglobin as a standard. The recombinant hemoglobin was detected in transfected plants using a rabbit anti-human hemoglobin antibody. No detectable cross-reacting protein was observed in the noninfected N. benthamiana control plants. The β-globin from transfected plants co-migrated with an authentic human standard and appears to form homodimers. This result suggests that rice α-amylase signal peptide was removed and that it may be possible to rapidly secrete functional hemoglobin in transfected plants.

### EXAMPLE 12

Construction of a Tobamoviral Vector for Expression of Heterologous Genes in A. thaliana.

Virions that were prepared as a crude aqueous extract of tissue from turnip infected with RMV were used to inoculate N. benthamiana, N. tabacum, A. thaliana, and oilseed rape (canola). Two to three weeks after transfection, systemically infected plants were analyzed by immunoblotting, using purified RMV as a standard. Total soluble plant protein concentrations were determined using bovine serum albumin as a standard. The proteins were analyzed on a 0.1% SDS/12.5% polyacrylamide gel and transferred by electroblotting for 1 hr to a nitrocellulose membrane. The blotted membrane was incubated for 1 hr with a 2000-fold dilution of anti-ribgrass mosaic virus coat antiserum. Using standard protocols, the antisera was raised in rabbits against purified RMV coat protein. The enhanced chemiluminescence horseradish peroxidase-linked, goat anti-rabbit IgG assay (Cappel Laboratories) was performed according to the manufacturer's (Amersham) specifications. The blotted membrane was subjected to film exposure times of up to 10 sec. No detectable cross-reacting protein was observed in the noninfected N. benthamiana control plant extracts. A 18 kDa protein cross-reacted to the anti-RMV coat antibody from systemically infected N. benthamiana, N. tabacum, A. thaliana, and oilseed rape (canola). This result demonstrates that RMV can systemically infect N. benthamiana, N. tabacum, A. thaliana, and oilseed rape (canola).

Plasmid Constructions.

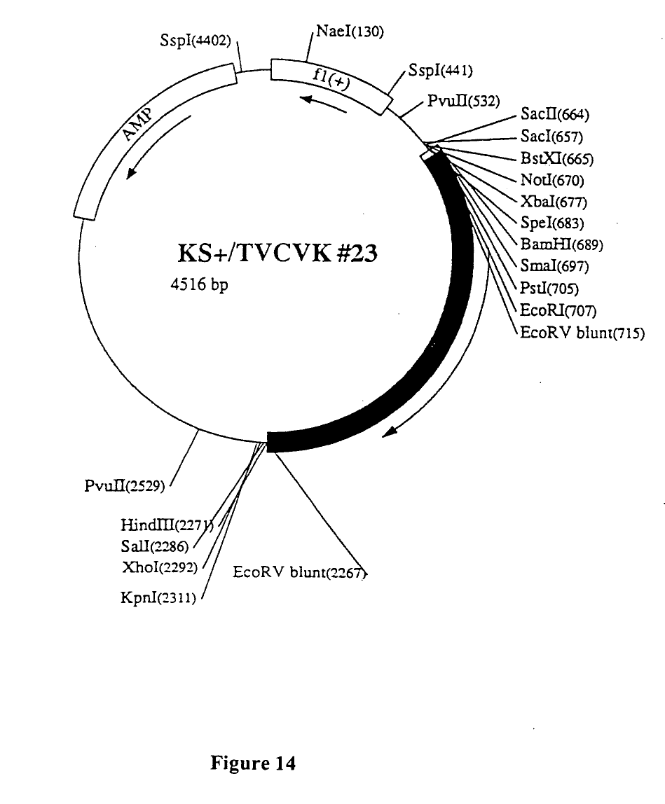

Ribgrass mosaic virus (RMV) is a member of the tobamovirus group that infects crucifers. A partial RMV cDNA containing the 30K subgenomic promoter, 30K ORF, coat subgenomic promoter, coat ORF, and 3′-end was isolated by RT-PCR using by using oligonucleotides TVCV183X 5′-TAC TCG AGG TTC ATA AGA CCG CGG TAG GCG G-3′ (upstream) (SEQ ID NO: 29) and TVCV KpnI 5′-CGG GGT ACC TGG GCC CCT ACC CGG GGT TTA GGG AGG-3′ (downstream) (SEQ ID NO: 30), and subcloned into the EcoRV site of KS+, creating plasmid KS+TVCV #23 (FIG. 14). The RMV cDNA was characterized by restriction mapping and dideoxy nucleotide sequencing. The partial nucleotide sequence is as follows:

| | |
|---|---|
| 5′-ctcgaggttcataagaccgcggtaggcggagcgtttgtttactgtagtataattaaatatttgtcagataaaaggttgtttaa | (SEQ ID NO: 31) |
| agatttgttttttgtttgactgagtcgataATGTCTTACGAGCCTAAAGTTAGTGACTTCCTTGCTC | |
| TTACGAAAAAGGAGGAAATTTTACCCAAGGCTTTGACGAGATTAAAGACTG | |
| TCTCTATTAGTACTAAGGATGTTATATCTGTTAAGGAGTCTGAGTCCCTGTG | |
| TGATATTGATTTGTTAGTGAATGTGCCATTAGATAAGTATAGGTATGTGGGT | |
| GTTTTGGGTGTTGTTTTCACCGGTGAATGGCTGGTACCGGATTTCGTTAAAG | |
| GTGGGGTAACAGTGAGCGTGATTGACAAACGGCTTGAAAATTCCAGAGAGT | |
| GCATAATTGGTACGTACCGAGCTGCTGTAAAGGACAGAAGGTTCCAGTTCA | |
| AGCTGGTTCCAAATTACTTCGTATCCATTGCGGATGCCAAGCGAAAACCGTG | |
| GCAGGTTCATGTGCGAATTCAAAATCTGAAGATCGAAGCTGGATGGCAACC | |
| TCTAGCTCTAGAGGTGGTTTCTGTTGCCATGGTTACTAATAACGTGGTTGTT | |
| AAAGGTTTGAGGGAAAAGGTCATCGCAGTGAATGATCCGAACGTCGAAGGT | |
| TTCGAAGGTGTGGTTGACGATTTCGTCGATTCGGTTGCTGCATTCAAGGCGA | |
| TTGACAGTTTCCGAAAGAAAAAGAAAAAGATTGGAggaagggatGTAAATAATA | |
| ATAAGTATAGATATAGACCGGAGAGATACGCCGGTCCTGATTCGTTACAAT | |
| ATAAAGAAGAAAaTGGTTTACAACATCACGAGCTCGAATCAGTACCAGTATT | |
| TCGCAGCGATGTGGGCAGAGCCCACAGCGATGCTTAAccaGTGCGTGTCTGC | |
| GTTGTCGCAATCGTATCAAACTCAGGCGGCAAgAGATACTGTTAGACAGCA | |
| GTTCTCTAACCTTCTGAGTGCGATTGTGACACCGAACCAGCGGTTTCCAgAA | |
| ACAGGATACCGGGTGTATATTAATTCAGCAGTTCTAAAACCGTTGTACGAGT | |
| CTCTCATGAAGTCCTTTGATACTAGAAATAGGATCATTGAAACTGAAGAAG | |
| AGTCGCGTCCATCGGCTTCCGAAGTATCTAATGCAACACAACGTGTTGATGA | |
| TGCGACCGTGGCCATCAGGAGTCAAATTCAGCTTTTGCTGAACGAGCTCTCC | |
| AACGGACATGGTCTGATGAACAGGGCAGAGTTCGAGGTTTTATTACCTTGG | |
| GCTACTGCGCCAGCTACATAGgcgtggtgcacacgatagtgcatagtgtttttctctccacttaaatcgaaga | |
| gatatacttacggtgtaattccgcaagggtggcgtaaaccaaattacgcaatgttttaggttccatttaaatcgaaacctgttatt | |
| tcctggatcacctgttaacgtacgcgtggcgtatattacagtgggaataactaaaagtgagaggttcgaatcctccctaaccccgg | |
| gtaggggccca-3′. | |

The 1543 base pair from the partial RMV cDNA was compared (PCGENE) to oilseed rape mosaic virus (ORMV). The nucleotide sequence identity was 97.8%. The RMV 30K and coat ORF were compared to ORMV and the amino acid identity was 98.11% (30K) and 98.73% (coat), respectively. A partial RMV cDNA containing the 5′-end and part of the replicase was isolated by RT-PCR from RMV RNA using by using oligonucleotides RGMV1 5′-GAT GGC GCC TTA ATA CGA CTC ACT ATA GTT TTA TTT TTG TTG CAA CAA CAA CAA C-3′ (upstream) (SEQ ID NO: 32) and RGR 132 5′-CTT GTG CCC TTC ATG ACG AGC TAT ATC ACG-3′ (downstream) (SEQ ID NO: 33). The RMV cDNA was characterized by dideoxy nucleotide sequencing. The partial nucleotide sequence containing the T7 RNA polymerase promoter and part of the RMV cDNA is as follows: 5 ′-ccttaatacgactcactataGTTTTATTTTTGTTGCAACAACAACAACAAATTACAATA ACAACAAAACAAATACAAACAACAACAACATGGCACAATTTCAACAAACA GTAAACATGCAAACATTGCAGGCTGCCGCAGGGCGCAACAGCCTGGTGAAT GATTTAGCCTCACGACGTGTTTATGACAATGCTGTCGAGGAGCTAAATGCAC GCTCGAGACGCCCTAAGGTTCATTACTCCAAATCAGTGTCTACGGAACAGA CGCTGTTAGCTTCAAACGCTTATCCGGAGTTTGAGATTTCCTTTACTCATACC CAACATGCCGTACACTCCCTTGCGGGTGGCCTAAGGACTCTTGAGTTAGAGT ATCTCATGATGCAAGTTCCGTTCGGTTCTCTGACGTACGACATCGGTGGTAA CTTTGCAGCGCACCTTTTCAAAGGACGCGACTACGTTCACTGCTGTATGCCA AACTTGGATGTACGTGATATAGCT-3′ (SEQ ID NO: 34). The uppercase letters are nucleotide sequences from RMV cDNA. The lower case letters are nucleotide sequences from T7 RNA polymerase promoter. The nucleotide sequences from the 5′ and 3′ oligonucleotides are underlined.

Full length infectious RMV cDNA clones were obtained by RT-PCR from RMV RNA using by using oligonucleotides RGMV1 5′-GAT GGC GCC TTA ATA CGA CTC ACT ATA GTT TTA TTT TTG TTG CAA CAA CAA CAA C-3′ (upstream) (SEQ ID NO: 35) and RG1 APE 5′-ATC GTT TAA ACT GGG CCC CTA CCC GGG GTT AGG GAG G-3′ (downstream) (SEQ ID NO: 36). The RMV cDNA was characterized by dideoxy nucleotide sequencing. The partial nucleotide sequence containing the T7 RNA polymerase promoter and part of the RMV cDNA is as follows: 5′-CCTTAATACGACTCACTATAGTTTTATTTTTGTTGCAACAACAACAACAA ATTACAATAACAACAAAACAAATACAAACAACAACAACATGGCACAATTTC AACAAACAGTAAACATGCAAACATTCCAGGCTGCCGCAGGGCGCAACAGCC TGGTGAATGATTTAGCCTCACGACGTGTTTATGACAATGCTGTCGAGGAGCT AAATGCACGCTCGAGACGCCCTAAGGTTCATTACTCCAAATCAGTGTCTACG GAACAGACGCTGTTAGCTTCAAACGCTTATCCGGAGTTTGAGATTTCCTTTA CTCATACCCAAACATGCCGTACACTCCCTTGCGGGTGGCCTAAGGACTCTTG AGTTAGAGTATCTCATGATGCAAGTTCCGTTCGGTTCTCTGACGTACGACAT CGGTGGTAACTTTGCAGCGCACCTTTTCAAAGGACGCGACTACGTTCACTGC TGTATGCCAAACTTGGATGTACGTGATATAGCT-3′ (SEQ ID NO: 37). The uppercase letters are nucleotide sequences from RMV cDNA. The nucleotide sequences from the 5′ and 3′ oligonucleotides are underlined. Full length infectious RMV cDNA clones were obtained by RT-PCR from RMV RNA using oligonucleotides RGMV1 5′-gat ggc gcc tta ata cga ctc act ata gtt tta ttt ttg ttg caa caa caa caa c-3′ (upstream) (SEQ ID NO: 38) and RG1 APE 5′-ATC GTT TAA ACT GGG CCC CTA CCC GGG GTT AGG GAG G-3′ (downstream) (SEQ ID NO: 39).

### Example 13

Arabidopsis thaliana cDNA Library Construction in a Dual Subgenomic Promoter Vector.

Arabidopsis thaliana cDNA libraries obtained from the Arabidopsis Biological Resource Center (ABRC). The four libraries from ABRC were size-fractionated with inserts of 0.5-1 kb (CD4-13), 1-2 kb (CD4-14), 2-3 kb (CD4-15), and 3-6 kb (CD4-16) All libraries are of high quality and have been used by several dozen groups to isolate genes. The pBluescript® phagemids from the Lambda ZAP II vector were subjected to mass excision and the libraries were recovered as plasmids according to standard procedures.

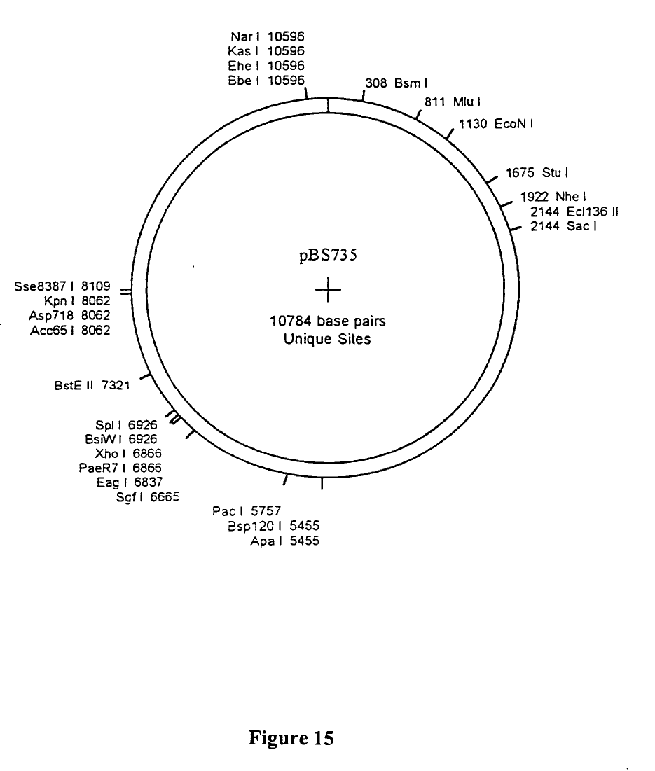

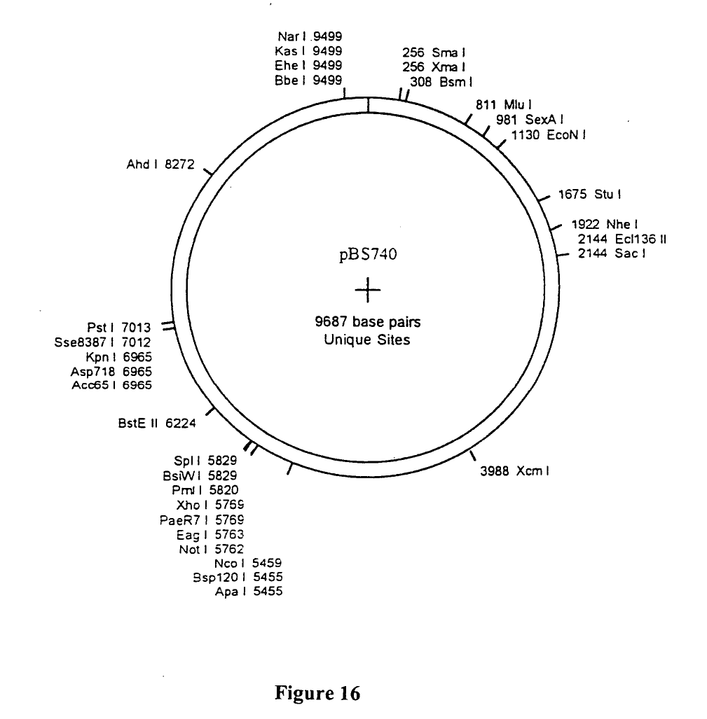

Alternatively, the cDNA inserts in the CD4-13 (Lambda ZAP II vector) were recovered by digestion with NotI. Digestion with NotI in most cases liberates the entire Arabidopsis thaliana cDNA insert because the original library was assembled with NotI adapters. NotI is an 8-base cutter that infrequently cleaves plant DNA. In order to insert the NotI fragments into a transcription plasmid, the pBS735 transcription plasmid (FIG. 15) was digested with PacI/XhoI and ligated to an adapter DNA sequence created from the oligonucleotides 5′-TCGAGCGGCCGCAT-3′ (SEQ ID NO: 40) and 5′-GCGGCCGC-3′ (SEQ ID NO: 41). The resulting plasmid pBS740 (FIG. 16) contains a unique NotI restriction site for bidirectional insertion of NotI fragments from the CD4-13 library. Recovered colonies were prepared from these for plasmid minipreps with a Qiagen BioRobot 9600®. The plasmid DNA preps performed on the BioRobot 9600® are done in 96-well format and yield transcription quality DNA. An Arabidopsis cDNA library was transformed into the plasmid and analyzed by agarose gel electrophoresis to identify clones with inserts. Clones with inserts may be transcribed in vitro and inoculated onto N. benthamiana and/or Arabidopsis thaliana. Selected leaf disks from transfected plants may be then taken for biochemical analysis.

### Example 14

Expression and Targeting to the Chloroplasts of a Green Fluorescent Protein in Arabidopsis thaliana via a Recombinant Viral Nucleic Acid Vector.

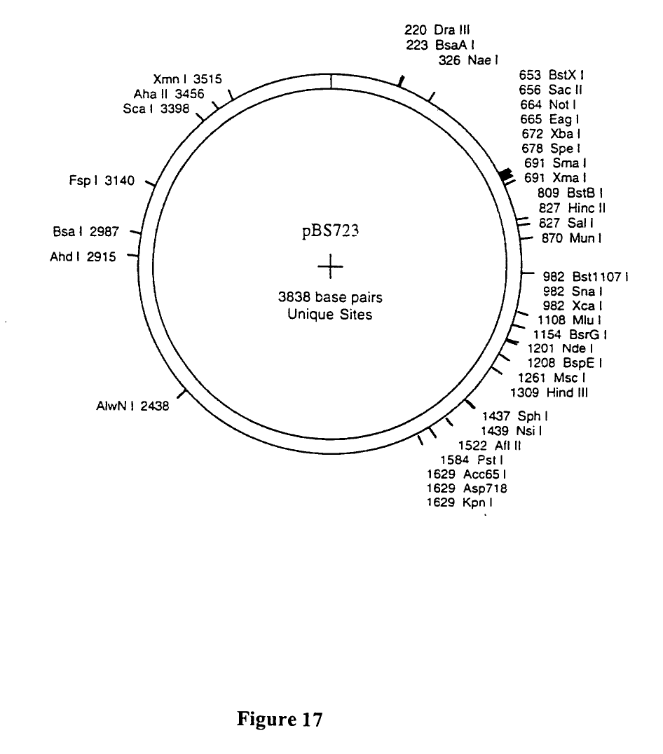

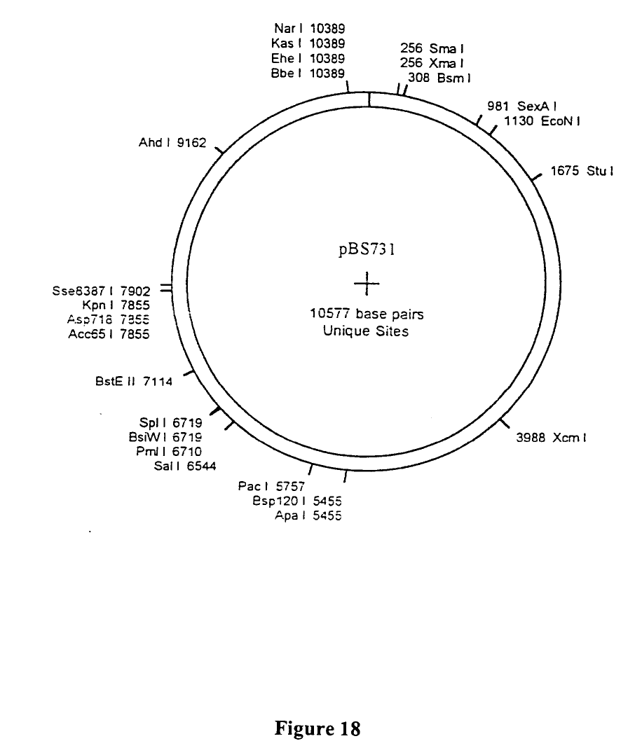

The gene encoding green fluorescent protein (GFP) was fused at the N-terminus to the chloroplast transit peptide (CTP) sequence of RuBPCase to create plasmid pBS723 (FIG. 17). Plasmid pBS723 was modified by PCR mutagenesis to create a unique PacI site upstream of the ATG start codon of the CTP-GFP gene fusion. The PCR amplification product obtained from plasmid pBS723 was digested PacI/Sall and cloned into plasmid GFP-30B/clone 60 (also digested with PacI/SalI) to create plasmid pBS731 (FIG. 18). Plasmid pBS731 was linearized at a unique KpnI restriction site and transcribed into infectious RNA with T7 RNA polymerase according to standard procedures. Infectious RNA transcripts that were inoculated onto Nicotiana benthamiana plants showed systemic expression in the upper leaves of CTP-GFP within six days. Plants infected with RNA transcripts from plasmid pBS731 were harvested by grinding the leaves with a mortar and pestle to obtain recombinant virions derived from pBS731 infectious RNA transcripts. Virions from pBS731 were inoculated onto Arabidopsis thaliana leaves. The inoculated leaves of Arabidopsis thaliana plants showed strong green fluorescence under UV light, thus indicating successful expression of the CTP-GFP reporter gene.

### Example 15

High Throughput Robotics.

Inoculation of subject organisms such as plants may be effected by using means of high throughput robotics. For example, Arabidopsis thaliana were grown in microtiter plates such as the standard 96-well and 384-well microtiter plates. A robotic handling arm then moved the plates containing the organism to a colony picker or other robot that may deliver inoculations to each plant in the well. By this procedure, inoculation was performed in a very high speed and high throughput manner. It is preferable in the case of plants that the organism be a germinating seed at least in the development cycle to enable access to the cells to be transfected. Equipment used for automated robotic production line could include, but not be limited to, robots of these types: electronic multichannel pipetmen, Qiagen BioRobot 9600®, Robbins Hydra liquid handler, Flexys Colony Picker, New Brunswick automated plate pourer, GeneMachines HiGro shaker incubator, New Brunswick floor shaker, three Qiagen BioRobots, MJ Research PCR machines (PTC-200, Tetrad), ABI 377 sequencer and Tecan Genesis RSP200 liquid handler.

### Example 16

Genomic DNA Library Cconstruction in a Recombinant Viral Nucleic Acid Vector.

Genomic DNA represented in BAC (bacterial artificial chromosome) or YAC (yeast artificial chromosome) libraries may be obtained from the Arabidopsis Biological Resource Center (ABRC). The BAC/YAC DNA can be mechanically size-fractionated, ligated to adapters with cohesive ends, and shotgun-cloned into recombinant viral nucleic acid vectors. Alternatively, mechanically size-fractionated genomic DNA can be blunt-end ligated into a recombinant viral nucleic acid vector. Recovered colonies can be prepared for plasmid minipreps with a Qiagen BioRobot 9600®. The plasmid DNA preps done on the BioRobot 9600® may be assembled in 96-well format and yield transcription quality DNA. The recombinant viral nucleic acid/Arabidopsis genomic DNA library may be analyzed by agarose gel electrophoresis (template quality control step) to identify clones with inserts. Clones with inserts can then be transcribed in vitro and inoculated onto N. benthamiana and/or Arabidopsis thaliana. Selected leaf disks from transfected plants can then be taken for biochemical analysis.

Genomic DNA from Arabidopsis typically contains a gene every 2.5 kb (kilobases) on average. Genomic DNA fragments of 0.5 to 2.5 kb obtained by random shearing of DNA were shotgun assembled in a recombinant viral nucleic acid expression/knockout vector library. Given a genome size of Arabidopsis of approximately 120,000 kb, a random recombinant viral nucleic acid genomic DNA library would need to contain minimally 48,000 independent inserts of 2.5 kb in size to achieve 1× coverage of the Arabidopsis genome. Alternatively, a random recombinant viral nucleic acid genomic DNA library would need to contain minimally 240,000 independent inserts of 0.5 kb in size to achieve 1× coverage of the Arabidopsis genome. Assembling recombinant viral nucleic acid expression/knockout vector libraries from genomic DNA rather than cDNA has the potential to overcome known difficulties encountered when attempting to clone rare, low-abundance mRNA's in a cDNA library. A recombinant viral nucleic acid expression/knockout vector library made with genomic DNA would be especially useful as a gene silencing knockout library. In addition, the DHSPES expression/knockout vector library made with genomic DNA would be especially useful for expression of genes lacking introns. Furthermore, other plant species with moderate to small genomes (e.g. rose, approximately 80,000 kb) would be especially useful for recombinant viral nucleic acid expression/knockout vector libraries made with genomic DNA. A recombinant viral nucleic acid expression/knockout vector library could be made from existing BAC/YAC genomic DNA or from newly-prepared genomic DNA for any plant species. Alternatively, a recombinant viral nucleic acid expression/knockout vector library could be made with genomic DNA obtained from yeast, bacteria, or animals including humans.

### Example 17

Genomic DNA or cDNA Library Construction in a DHSPES Vector, and Transfection of Individual Clones from said Vector Library onto T-DNA Tagged or Transposon Tagged or Mutated Plants.

Genomic DNA or cDNA library construction in a recombinant viral nucleic acid vector, and transfection of individual clones from the vector library onto T-DNA tagged or transposon tagged or mutated plants may be performed according the procedure set forth in Example 16. Such a protocol may be easily designed to complement mutations introduced by random insertional mutagenesis of T-DNA sequences or transposon sequences.

### Example 18

Production of a Malarial CTL Epitope Genetically Fused to the C Terminus of the TMVCP.

Malarial immunity induced in mice by irradiated sporozites of P. yoelii is also dependent on CD8+ T lymphocytes. Clone B is one ocytotoxic T lymphocyte (CTL) cell clone shown to recognize an epitope present in both the P. yoelii and P. berghei CS proteins. Clone B recognizes the following amino acid sequence; SYVPSAEQILEFVKQISSQ (SEQ ID NO: 42) and when adoptively transferred to mice protects against infection from both species of malaria sporozoites. Construction of a genetically modified tobamovirus designed to carry this malarial CTL epitope fused to the surface of virus particles is set forth herein.

Construction of plasmid pBGC289. A 0.5 kb fragment of pBGC11 was PCR amplified using the 5′ primer TB2ClaI5′ and the 3′ primer C/-5AvrII. The amplified product was cloned into the SmaI site of pBstKS+ (Stratagene Cloning Systems) to form pBGC214.

PBGC215 was formed by cloning the 0.15 kb AccI-NsiI fragment of pBGC214 into pBGC235. The 0.9 kb NcoI-KpnI fragment from pBGC215 was cloned in pBGC152 to form pBGC216.

A 0.07 kb synthetic fragment was formed by annealing PYCS.2p with PYCS.2m and the resulting double stranded fragment, encoding the P. yoelii CTL malarial epitope, was cloned into the AvrII site of pBGC215 made blunt ended by treatment with mung bean nuclease and creating a unique AatII site, to form pBGC262. A 0.03 kb synthetic AatII fragment was formed by annealing TLS.1EXP with TLS.1 EXM and the resulting double stranded fragment, encoding the leaky-stop sequence and a stuffer sequence used to facilitate cloning, was cloned into AatII digested pBGC262 to form pBGC263. PBGC262 was digested with AatII and ligated to itself removing the 0.02 kb stuffer fragment to form pBGC264. The 1.0 kb NcoI-KpnI fragment of pBGC264 was cloned into pSNC004 to form pBGC289.

The virus TMV289 produced by transcription of plasmid pBGC289 in vitro contains a leaky stop signal resulting in the removal of four amino acids from the C terminus of the wild type TMV coat protein gene and is therefore predicted to synthesize a truncated coat protein and coat protein with a CTL epitope fused at the C terminus at a ratio of 20:1. The recombinant TMVCP/CTL epitope fusion present in TMV289 is with the stop codon decoded as the amino acid Y (amino acid residue 156). The amino acid sequence of the coat protein of virus TMV216 produced by transcription of the plasmid pBGC216 in vitro, is truncated by four amino acids. The epitope SYVPSAEQILEFVKQISSQ (SEQ ID NO: 42) is calculated to be present at approximately 0.5% of the weight of the virion using the same assumptions confirmed by quantitative ELISA analysis.

Propagation and purification of the epitope expression vector. Infectious transcripts were synthesized from KpnI-linearized pBGC289 using T7 RNA polymerase and cap (7mGpppG) according to the manufacturer (New England Biolabs).

An increased quantity of recombinant virus was obtained by passaging Sample ID No. TMV289.11B1a. Fifteen tobacco plants were grown for 33 days post inoculation accumulating 595 g fresh weight of harvested leaf biomass not including the two lower inoculated leaves. Purified Sample ID No. TMV289.11B2 was recovered (383 mg) at a yield of 0.6 mg virion per gram of fresh weight. Therefore, 3 g of 19-mer peptide was obtained per gram of fresh weight extracted. Tobacco plants infected with TMV289 accumulated greater than 1.4 micromoles of peptide per kilogram of leaf tissue.

Product analysis. Partial confirmation of the sequence of the epitope coding region of TMV289 was obtained by restriction digestion analysis of PCR amplified cDNA using viral RNA isolated from Sample ID No. TMV289.11B2. The presence of proteins in TMV289 with the predicted mobility of the cp fusion at 20 kD and the truncated cp at 17.1 kD was confirmed by denaturing polyacrylamide gel electrophoresis.

### Example 19

Identification of Nucleotide Sequences Involved in the Regulation of Plant Growth by Cytoplasmic Inhibition of Gene Expression Using Viral Derived RNA.

Antisense RNA has been used to down regulate gene expression in transgenic and transfected plants. The effectiveness of antisense on the inhibition of eukaryotic gene expression was first demonstrated by Izant et al. (Cell 36(4):1007-1015 (1984)). Since then, the down-regulation of numerous genes from transgenic plants has been reported. In addition, there is evidence that “co-suppression” of genes occurs in transgenic plants containing sense RNA by readthrough transcription from distal promoters located on the opposite strand of the DNA (Van der Krol et al., Plant Cell 2(4):291-299 (1990) and Napoli et al., Plant Cell 2:279-289 (1990)).

In this example and examples 20 and 21, we show: (1) a novel method for producing sense/antisense RNA using an RNA viral vector, (2) a process to produce viral-derived sense/antisense RNA in the cytoplasm, (3) a process to inhibit the expression of endogenous plant proteins in the cytoplasm by viral antisense RNA, (4) a process to “co-suppress” the expression of endogenous plant proteins in the cytoplasm by viral RNA, and (5) a process to produce transfected plants containing viral antisense RNA which is much faster than the time required to obtain genetically engineered antisense transgenic plants. Systemic infection and expression of viral antisense RNA occurs as short as four days post inoculation, whereas it takes several months or longer to create a single transgenic plant. This example demonstrates that novel positive strand viral vectors, which replicate solely in the cytoplasm, can be used to identify genes involved in the regulation of plant growth by inhibiting the expression of specific endogenous genes. This example will enable one to characterize specific genes and biochemical pathways in transfected plants using an RNA viral vector.

Tobamoviral vectors have been developed for the heterologous expression of uncharacterized nucleotide sequences in transfected plants. A partial Arabidopsis thaliana cDNA library was placed under the transcriptional control of a tobamovirus subgenomic promoter in a RNA viral vector. Colonies from transformed E. coli were automatically picked using a Flexys robot and transferred to a 96 well flat bottom block containing terrific broth (TB) Amp 50 ug/ml. Approximately 2000 plasmid DNAs were isolated from overnight cultures using a BioRobot and infectious RNAs from 430 independent clones were directly applied to plants. One to two weeks after inoculation, transfected Nicotiana benthamiana plants were visually monitored for changes in growth rates, morphology, and color. One set of plants transfected with 740 AT #120 were severely stunted. DNA sequence analysis revealed that this clone contained an Arabidopsis GTP binding protein open reading frame (ORF) in the antisense orientation. This demonstrates that an episomal RNA viral vector can be used to deliberately manipulate a signal transduction pathway in plants. In addition, our results suggest that the Arabidopsis antisense transcript can turn off the expression of the N. benthamiana gene.

Construction of an Arabidopsis thaliana cDNA Library in an RNA Viral Vector.

An Arabidopsis thaliana CD4-13 cDNA library was digested with NotI. DNA fragments between 500 and 1000 bp were isolated by trough elution and subcloned into the NotI site of pBS740. E. coli C600 competent cells were transformed with the pBS740 AT library and colonies containing Arabidopsis cDNA sequences were selected on LB Amp 50 ug/ml. Recombinant C600 cells were automatically picked using a Flexys robot and then transferred to a 96 well flat bottom block containing terrific broth (TB) Amp 50 ug/ml. Approximately 2000 plasmid DNAs were isolated from overnight cultures using a BioRobot (Qiagen) and infectious RNAs from 430 independent clones were directly applied to plants.

Isolation of a Gene Encoding a GTP Binding Protein.

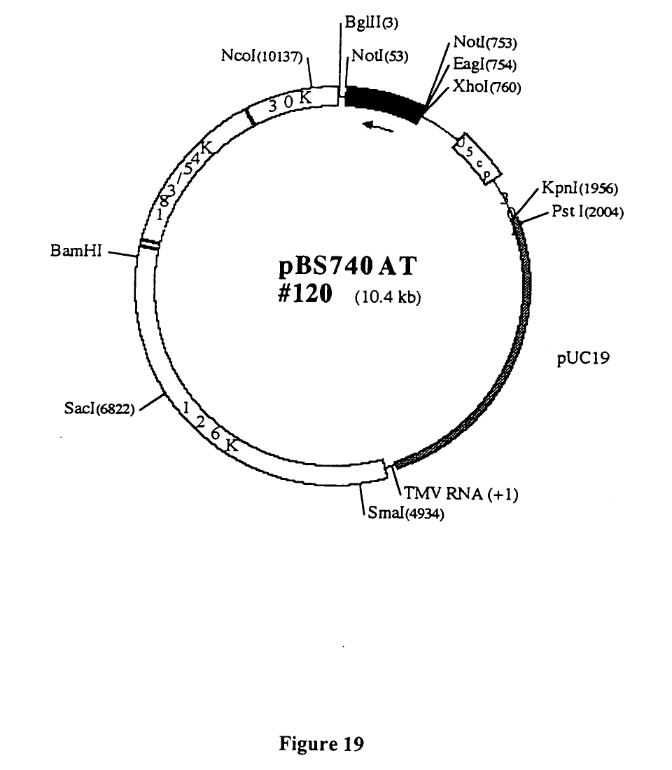

One to two weeks after inoculation, transfected Nicotiana benthamiana plants were visually monitored for changes in growth rates, morphology, and color. Plants transfected with 740 AT #120 (FIG. 19) were severely stunted.

DNA Sequencing and Computer Analysis.

A 782 bp NotI fragment of 740 AT #120 containing the ADP-ribosylation factor (ARF) cDNA was characterized. DNA sequence of NotI fragment of 740 AT #120 (774 base pairs) is as follows:

| | |
|---|---|
| 5′-CCGAAACATTCTTCGTAGTGAAGCAAAATGGGGTTGAGTTTCGCCAAGCT | (SEQ ID NO: 43) |
| GTTTAGCAGGCTTTTTGCCAAGAAGGAGATGCGAATTCTGATGGTTGGTCTT | |
| GATGCTGCTGGTAAGACCACAATCTTGTACAAGCTCAAGCTCGGAGAGATT | |
| GTCACCACCATCCCTACTATTGGTTTCAATGTGGAAACTGTGGAATACAAGA | |
| ACATTAGTTTCACCGTGTGGGATGTCGGGGGTCAGGACAAGATCCGTCCCTT | |
| GTGAGACACTACTTCCAGAACACTCAAGGTCTAATCTTTGTTGTTGATAGCA | |
| ATGACAGAGACAGAGTTGTTGAGGCTCGAGATGAACTCCACAGGATGCTGA | |
| ATGAGGACGAGCTGCGTGATGCTGTGTTGCTTGTGTTTGCCAACAAGCAAG | |
| ATCTTCCAAATGCTATGAACGCTGCTGAAATCACAGATAAGCTTGGCCTTCA | |
| CTCCCTCCGTCAGCGTCATTGGTATATCCAGAGCACATGTGCCACTTCAGGT | |
| GAAGGGCTTTATGAAGGTCTGGACTGGCTCTCCAACAACATCGCTGGCAAG | |
| GCATGATGAGGGAGAAATTGCGTTGCATCGAGATGATTCTGTCTGCTGTGTT | |
| GGGATCTCTCTCTGTCTTGATGCAAGAGAGATTATAAATATTATCTGAACCT | |
| TTTTGCTTTTTTGGGTATGTGAATGTTTCTTATTGTGCAAGTAGATGGTCTTG | |
| TACCTAAAAATTTACTAGAAGAACCCTTTTAAATAGCTTTCGTGTATTGT-3′. | |

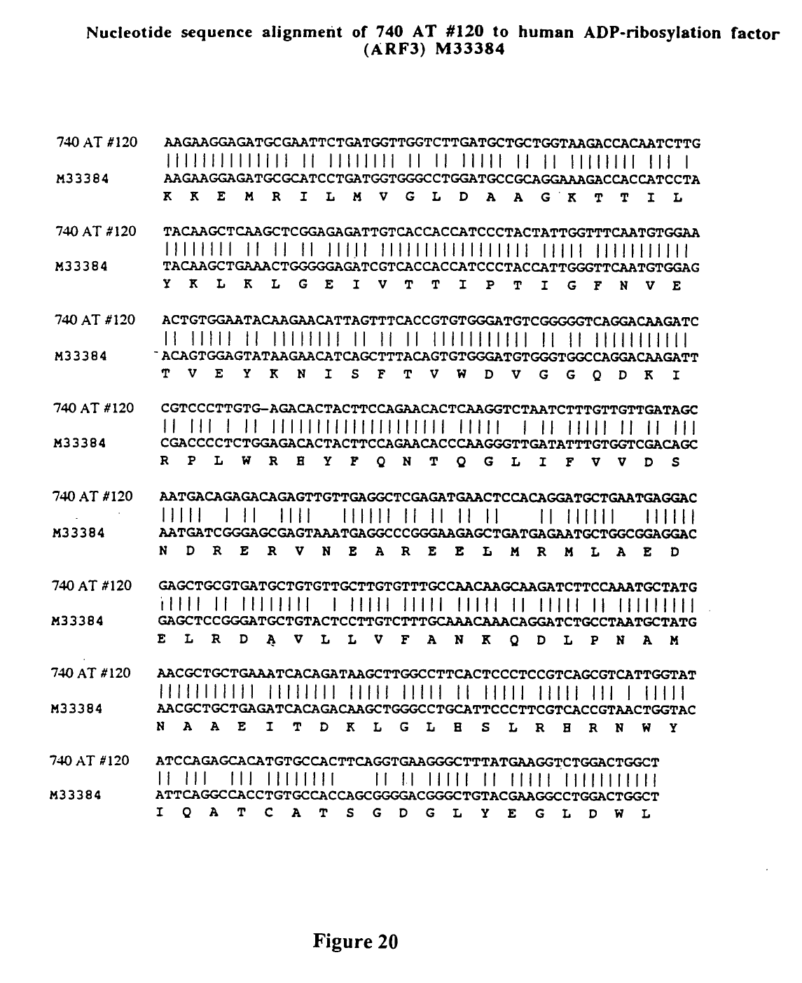

The nucleotide sequencing of 740 AT #120 was carried out by dideoxy termination using double stranded templates (Sanger et al., Proc. Natl. Acad. Sci. USA 74(12):5463-5467 (1977)). Nucleotide sequence analysis and amino acid sequence comparisons were performed using DNA Strider, PCGENE and NCBI Blast programs. The nucleotide sequence from 740 AT #120 was compared the human ADP-ribosylation factor (ARF3) W3384 (FIG. 20).

Isolation of a cDNA Encoding Nicotiana benthamiana ADP-ribosylation Factor.

Partial cDNAs from Nicotiana benthamiana leaf RNA may be isolated by polymerase chain reaction (PCR) using the following oligonucleotides: ATARFM1X, 5′-GCC TCG AGT GCA GCA TGG GGT TGT CAT TCG GAA AGT TGT TC-3′ (upstream) (SEQ ID NO: 44) and ATARFA181A, 5′-TAC CTA GGC CTT GCT TGC GAT GTT GTT GGA GAG-3′ (downstream) (SEQ ID NO: 45). A full-length cDNA encoding ARF may be isolated by screening a cDNA library by colony hybridization using a ³²P labeled Arabidopsis thaliana ARF PCR product. Hybridization can be carried out at 42° C. for 48 h in 50% formamide, 5×SSC, 0.02M phosphate buffer, 5× Denhart's solution, and 0.1 mg/ml sheared calf thymus DNA. Filters may be washed at 65° C. in 0.1×SSC and 0.1% SDS, prior to autoradiography. PCR products and the ARF cDNA clones may be verified by dideoxynucleotide sequencing.

### Example 20

Identification of Nucleotide Sequences Involved in the Regulation of Plant Development by Cytoplasmic Inhibition of Gene Expression Using Viral Derived RNA.

This example again demonstrates that an episomal RNA viral vector can be used to deliberately manipulate a signal transduction pathway in plants. In addition, our results suggest that the Arabidopsis antisense transcript can turn off the expression of the N. benthamiana gene.

A partial Arabidopsis thaliana cDNA library was placed under the transcriptional control of a tobamovirus subgenomic promoter in a RNA viral vector. Colonies from transformed E. coli were automatically picked using a Flexys robot and transferred to a 96 well flat bottom block containing terrific broth (TB) Amp 50 ug/ml. Approximately 2000 plasmid DNAs were isolated from overnight cultures using a BioRobot and infectious RNAs from 430 independent clones were directly applied to plants. One to two weeks after inoculation, transfected Nicotiana benthamiana plants were visually monitored for changes in growth rates, morphology, and color. One set of plants transfected with 740 AT #88 developed a white phenotype on the infected leaf tissue. DNA sequence analysis revealed that this clone contained an Arabidopsis G-protein coupled receptor open reading frame (ORF) in the antisense orientation.

Construction of an Arabidopsis thaliana cDNA Library in an RNA Viral Vector.

An Arabidopsis thaliana CD4-13 cDNA library was digested with NotI. DNA fragments between 500 and 1000 bp were isolated by trough elution and subcloned into the NotI site of pBS740. E. coli C600 competent cells were transformed with the pBS740 AT library and colonies containing Arabidopsis cDNA sequences were selected on LB Amp 50 ug/ml. Recombinant C600 cells were automatically picked using a Flexys robot and then transferred to a 96 well flat bottom block containing terrific broth (TB) Amp 50 ug/ml. Approximately 2000 plasmid DNAs were isolated from overnight cultures using a BioRobot (Qiagen) and infectious RNAs from 430 independent clones were directly applied to plants.

Isolation of a Gene Encoding a G-Protein Coupled Receptor.

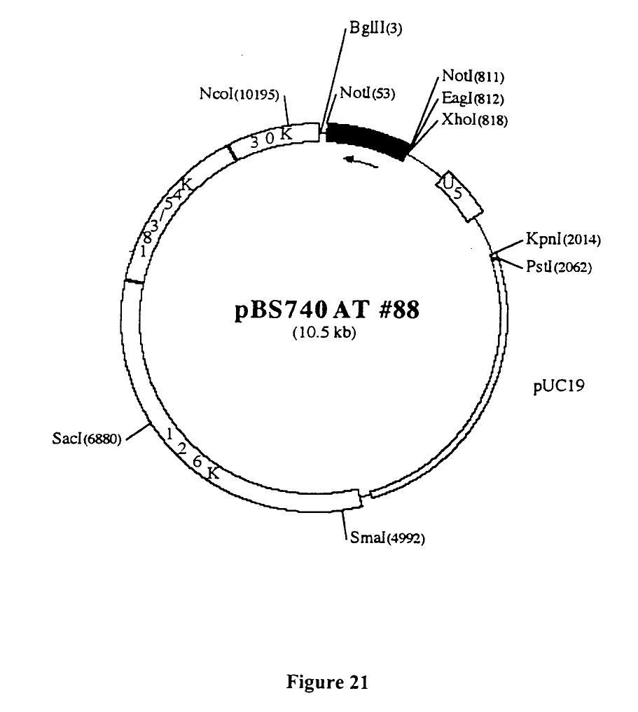

One to two weeks after inoculation, transfected Nicotiana benthamiana plants were visually monitored for changes in growth rates, morphology, and color. Plants transfected with 740 AT #88 (FIG. 21) developed a white phenotype on the infected leaf tissue.

DNA Sequencing and Computer Analysis.

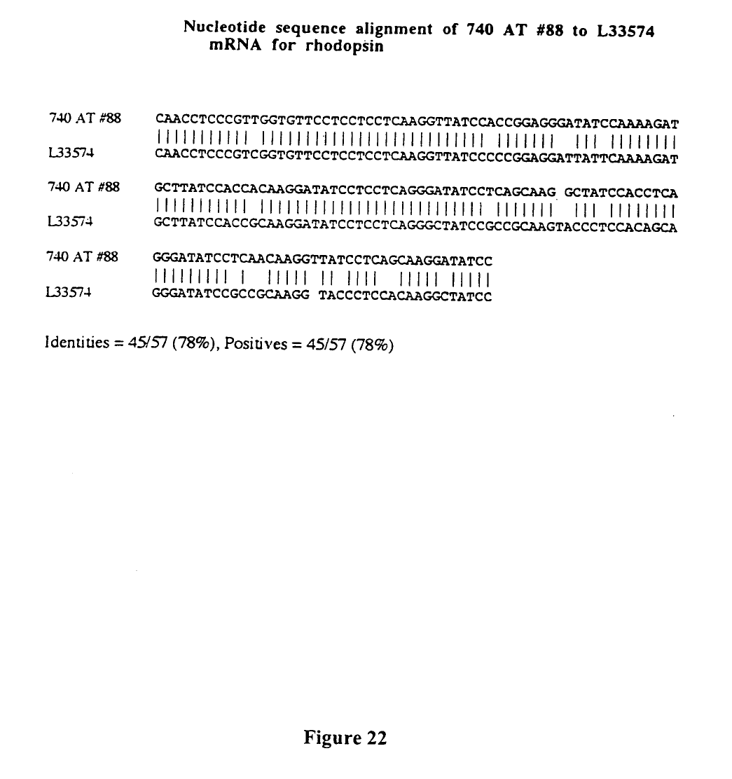

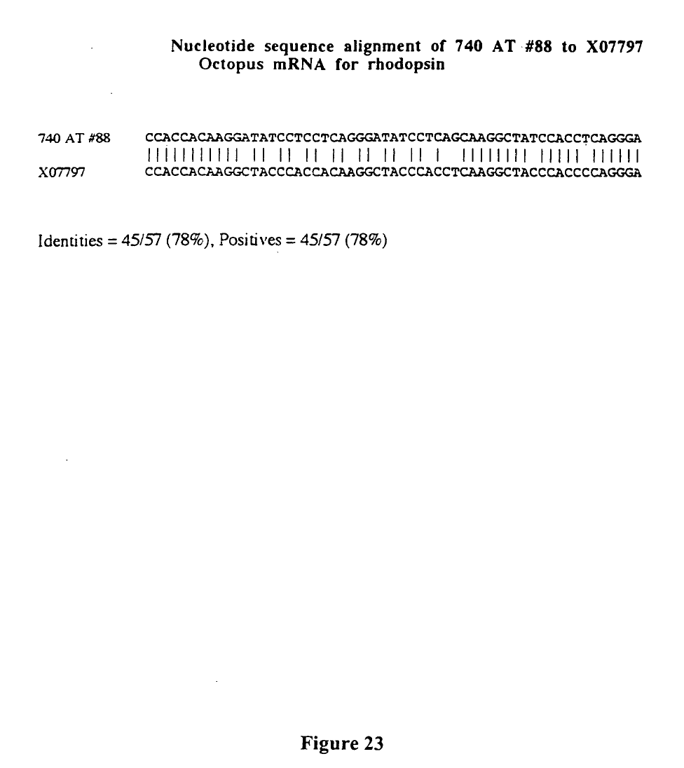

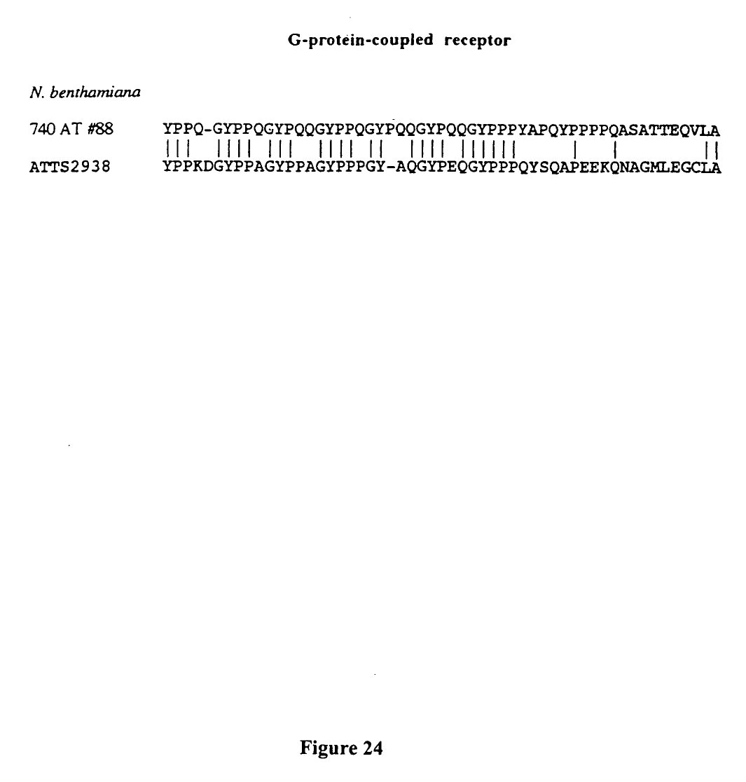

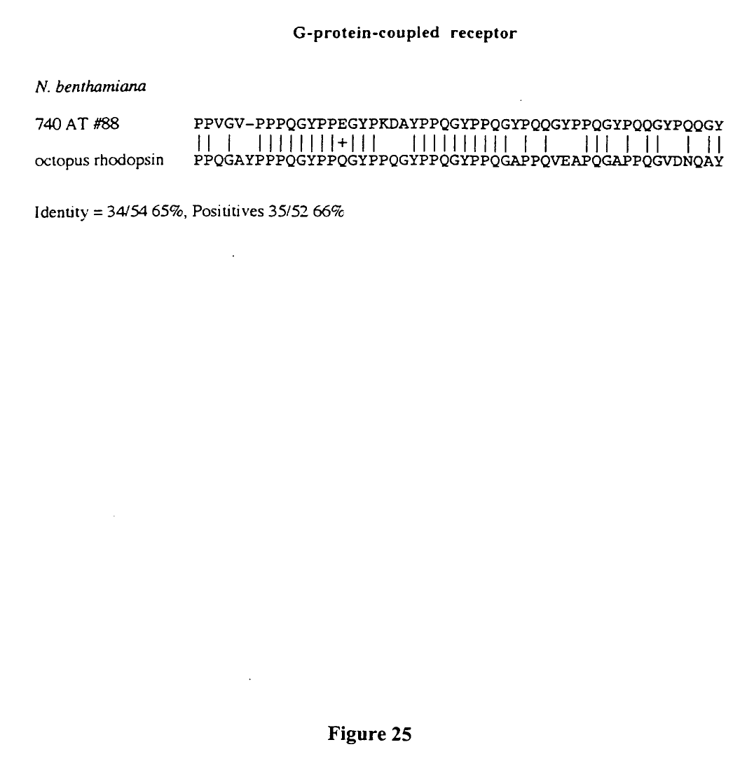

A 750 bp NotI fragment of 740 AT #88 containing the G-protein coupled receptor cDNA was characterized. DNA sequence of NotI fragment of 740 AT #88 (750 bp) is as follows: 5′-TTTCGATCTAAGGTTCGTGATCTCCTTCTTCTCTACGAAGTTTACACTTTTT CTTCAAAGGAAACAATGAGCCAGTACAATCAACCTCCCGTTGGTGTTCCTCC TCCTCAAGGTTATCCACCGGAGGGATATCCAAAAGATGCTTATCCACCACA AGGATATCCTCCTCAGGGATATCCTCAGCAAGGCTATCCACCTCAGGGATAT CCTCAACAAGGTTATCCTCAGCAAGGATATCCTCCACCGTACGCGCCTCAAT ATCCTCCACCACCGCAAGCATCAGCAACAACAGAGCAAGTCCTGGCTTTCT AGAAGGATGTCTTGCTGCTCTGTGTTGTTGCTGTCTCTTGGATGCTTGCTTCT GATTGGAGTCTCTCTCTCTCTGCATAAAGCTTCGGGATTTATTTGTAAGAGG GTTTTTGGGTTAAACAAAAACCTTAATTGATTTGTGGGGCATTAAAAATGAA TCTCTCGATGATTCTCTTCGTTTATGTGGTAATGTTCTTCGGTTATAACATTT AACATTGCTATCGACGTTCTGCCTAGTTGGATTTGATTATTGGGAATGTAAA TTGGTTGGGAAGACACCGGGCCGTTAATGACAGAACCCGAACTGAGATGGA GTATGATCTGAAATATTTAAAACAATCCTCGCGACATAGCCTCCAATCTCAT CGTAAATATTCTTTTTAAACTATTCCCAATCTTAACTTTTATAGTCTGGTCGA CTGACCACTACTCTTTTTCCTT-3′ (SEQ ID NO: 46). The nucleotide sequencing of 740 AT #88 was carried out by dideoxy termination using double stranded templates (Sanger et al., Proc. Natl. Acad. Sci. USA 74(12):5463-5467 (1977)). Nucleotide sequence analysis and amino acid sequence comparisons were performed using DNA Strider, PCGENE and NCBI Blast programs. The nucleotide sequence from 740 AT #88 was compared to Brassica rapa cDNA L33574 (FIG. 22), the octopus rhodopsin mRNA X07797 (FIG. 23). The amino acid sequence derived from 740 AT #88 was compared to an Arabidopsis EST ORF ATTS2938 (FIG. 24) and octopus rhodopsin P31356 (FIG. 25).

### Example 21

Identification of Nucleotide Sequences Involved in the Regulation of Plant Growth by Cytoplasmic Inhibition of Gene Expression Using Viral Derived RNA.

Antisense RNA has been used to down regulate gene expression in transgenic and transfected plants. The purpose of this example is again to demonstrate that novel positive strand viral vectors, which replicate solely in the cytoplasm, can be used to identify genes involved in the regulation of plant growth by inhibiting the expression of specific endogenous genes. This example will enable one to characterize specific genes and biochemical pathways in transfected plants using an RNA viral vector.

The protocols of this example are analogous to those of examples 19 and 20. Tobamoviral vectors have been developed for the heterologous expression of uncharacterized nucleotide sequences in transfected plants. A partial Arabidopsis thaliana cDNA library was placed under the transcriptional control of a tobamovirus subgenomic promoter in a RNA viral vector. Colonies from transformed E. coli were automatically picked using a Flexys robot and transfered to a 96 well flat bottom block containing terrific broth (TB) Amp 50 ug/ml. Approximately 2000 plasmid DNAs were isolated from overnight cultures using a BioRobot and infectious RNAs from 430 independent clones were directly applied to plants. One to two weeks after inoculation, transfected Nicotiana benthamiana plants were visually monitored for changes in growth rates, morphology, and color. One set of plants transfected with 740 AT #2441 developed white leaves and were severely stunted. DNA sequence analysis revealed that this clone contained an Arabidopsis GTP binding protein open reading frame (ORF) in the positive orientation. This demonstrates that an episomal RNA viral vector can be used to deliberately manipulate a signal transduction pathway in plants.

Construction of an Arabidopsis thaliana cDNA library in an RNA viral vector. An Arabidopsis thaliana CD4-13 cDNA library was digested with NotI. DNA fragments between 500 and 1000 bp were isolated by trough elution and subcloned into the NotI site of pBS740. E. coli C600 competent cells were transformed with the pBS740 AT library and colonies containing Arabidopsis cDNA sequences were selected on LB Amp 50 ug/ml. Recombinant C600 cells were automatically picked using a Flexys robot and then transfered to a 96 well flat bottom block containing terrific broth (TB) Amp 50 ug/ml. Approximately 2000 plasmid DNAs were isolated from overnight cultures using a BioRobot (Qiagen) and infectious RNAs from 430 independent clones were directly applied to plants.

Isolation of a gene encoding a GTP binding protein. One to two weeks after inoculation, transfected Nicotiana benthamiana plants were visually monitored for changes in growth rates, morphology, and color. Plants transfected with 740 AT #2441 developed white leaves and were severely stunted.

DNA sequencing and computer analysis. A NotI fragment of 740 AT #2441 containing the RAN GTP binding protein ORF cDNA was characterized. DNA sequence of NotI fragment of 740 AT #2441 (350 bp) is as follows: 5′-

| | |
|---|---|
| CTTCACTTTCGCCGATGGCTCTACCTAACCAGCAAACCGTGGATTACCCTAG | (SEQ ID NO. 47) |
| CTTCAAGCTCGTTATCGTTGGCGATGGAGGCACAGGGAAGACCACATTTGT | |
| AAAGAGACATCTTACTGGAGAGTTTGAGAAGAAGTATGAACCCACTATTGG | |
| TGTTGAGGTTCATCCTCTTGATTTCTTCACTAACTGTGGCAAGATCCGTTTCT | |
| ACTGTTGGGATACTGCTGGCCAAGAGAAATTTGGTGGTCTTAGGGATGGTTA | |
| CTACATCCATGGACAATGTGCTATCATCATGTTTGATGTCACAAGCACGACT | |
| GACATACAAGAATGTTCCAACATGGCACCGTGATCTTTG-3′. | |

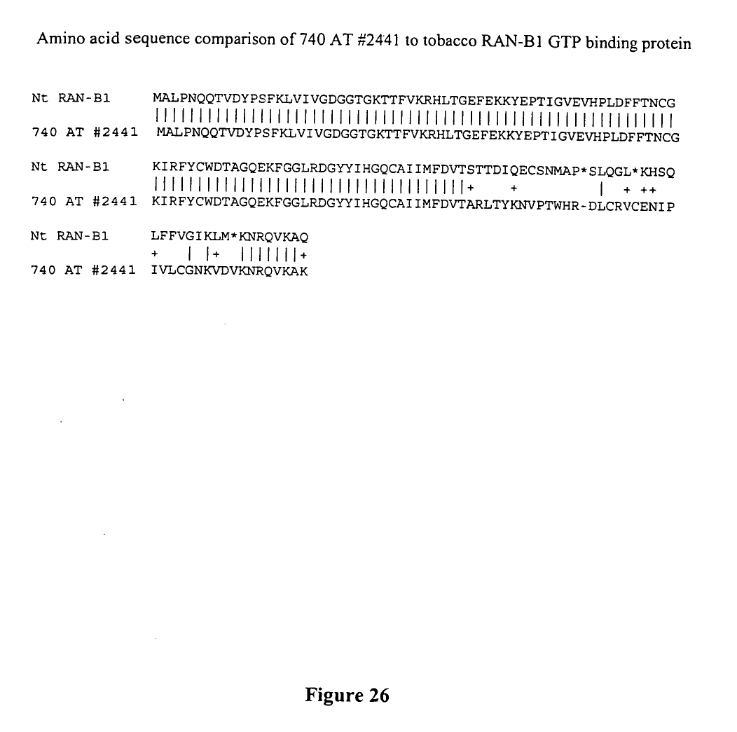

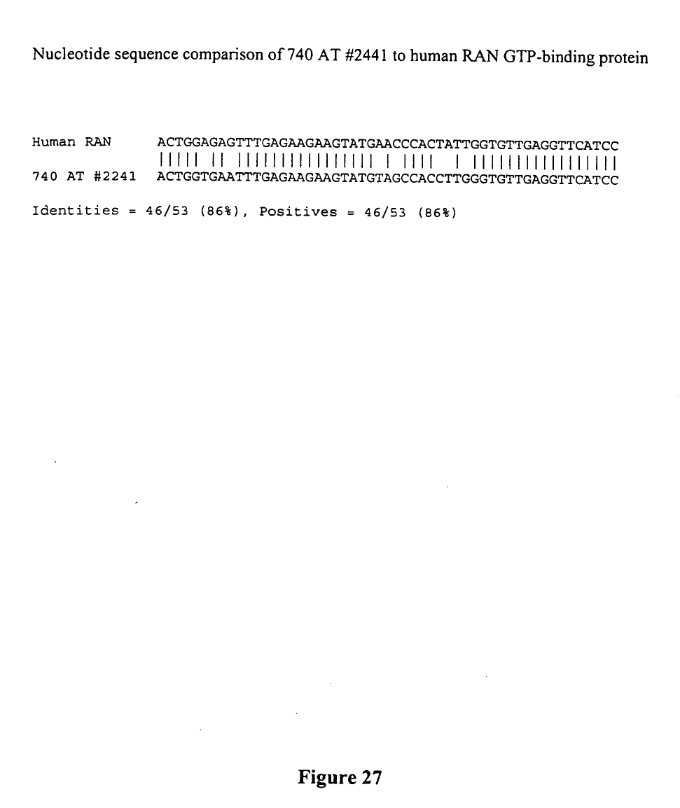

The nucleotide sequencing of 740 AT #2441 was carried out by dideoxy termination using double stranded templates (Sanger et al., Proc. Natl. Acad. Sci. USA 74(12):5463-5467 (1977)). Nucleotide sequence analysis and amino acid sequence comparisons were performed using DNA Strider, PCGENE and NCBI Blast programs. The nucleotide sequence from 740 AT #2441 was compared to tobacco RAN-B1 GTP binding protein (FIG. 26). The nucleotide sequence from 740 AT #2441 was compared to human RAN GTP-binding protein (FIG. 27).

### Example 22

Gene Silencing/Co-Supression of Genes Induced by Delivering an RNA Capable of Base Pairing with Itself to Form Double Stranded Regions.

Gene silencing has been used to down regulate gene expression in transgenic plants. Recent experimental evidence suggests that double stranded RNA may be an effective stimulator of gene silencing/co-suppression phenomenon in transgenic plant. For example, Waterhouse et al. (Proc. Natl. Acad. Sci. USA 95:13959-13964 (1998), incorporated herein by reference) described that virus resistance and gene silencing in plants could be induced by simultaneous expression of sense and antisense RNA. Gene silencing/co-suppression of plant genes may be induced by delivering an RNA capable of base pairing with itself to form double stranded regions.

This example shows: (1) a novel method for generating an RNA virus vector capable of producing an RNA capable of forming double stranded regions, and (2) a process to silence plant genes by using such a viral vector.

Step 1: Construction of a DNA sequence which after it is transcribed would generate an RNA molecule capable of base pairing with itself. Two identical, or nearly identical, ds DNA sequences can be ligated together in an inverted orientation to each other (i.e., in either a head to tail or tail to head orientation) with or without a linking nucleotide sequence between the homologous sequences. The resulting DNA sequence can then be cloned into a cDNA copy of a plant viral vector genome.

Step 2: Cloning, screening, transcription of clones of interest using known methods in the art.

Step 3: Infect plant cells with transcripts from clones.

As virus expresses foreign gene sequence, RNA from foreign gene will base pair upon itself, forming double-stranded RNA regions. This approach could be used with any plant or non-plant gene and used to silence plant gene homologous to assist in identification of the function of a particular gene sequence.

### Example 23

Preparation of a Non-Infective Eastern Equine Encephalomyelitis Virus Nucleotide Sequence.

Methods for genetic manipulation of Eastern Equine Encephalomyelitis Virus are described in Garoffet al., Curr. Opin. Biotechnol. 9(5):464-9 (1998); Pushko et al., Virology 239(2):389-401 (1997); and Davis et al., J. Virol. 70(6):3781-7 (1996), all of which are incorporated herein by reference. A full-length cDNA copy of the Eastern Equine Encephalomyelitis Virus (EEEV) genome is prepared and inserted into the PstI site of pUC18 as described by Chang et al., J. Gen. Virol. 68:2129 (1987). The sequence for the viral coat protein and its adjacent E1 and E2 glycoprotein transmissibility factors are located on the region corresponding to the 26S RNA region. The vector containing the cDNA copy of the EEEV genome is digested with the appropriate restriction enzymes and exonucleases to delete the coding sequence of the coat protein and the E1 and E2 proteins (structural protein coding sequence).

For example, the structural protein coding sequence is removed by partial digestion with MboI, followed by religation to remove a vital portion of the structural gene. Alternatively, the vector is cut at the 3′-end of the viral structural gene. The viral DNA is sequentially removed by digestion with Bal31 or Micrococcal S1 nuclease up through the start codon of the structural protein sequence. The DNA sequence containing the sequence of the viral 3′-tail is then ligated to the remaining 5′-end. The deletion of the coding sequence for the structural proteins is confinned by isolating EEEV RNA and using it to infect an equine cell culture. The isolated EEEV RNA is found to be non-infective under natural conditions.

Alternatively, only the coding sequence for the coat protein is deleted and the sequence for the E1 and E2 glycoproteins remain in the vector containing the cDNA copy of the EEEV genome. In this case, the coat protein coding sequence is removed by partial digestion with MboI followed by religation to reattach the 3′-tail of the virus. This will remove a vital portion of the coat protein gene.

A second alternative method for removing only the coat protein sequence is to cut the vector at the 3′-end of the viral coat protein gene. The viral DNA is removed by digestion with Bal31 or Micrococcal S1 nuclease up through the start codon of the coat protein sequence. The synthetic DNA sequence containing the sequence of the 3′-tail is then ligated to the remaining 5′-end.

The deletion of the coding sequence for the coat protein is confirmed by isolating EEEV RNA and using it to infect an equine cell culture. The isolated EEEV RNA is found to be non-infective under natural conditions.

### Example 24

Preparation of a Non-Transmissible Sindbis Virus Nucleotide Sequence.

Methods for genetic manipulation of Sindbis viruses are described in Garoff et al., Curr. Opin. Biotechnol. 9(5):464-9 (1998); Agapov et al., Proc. Natl. Acad. Sci. USA 95(22):12989-94 (1998); Frolov et al., J. Virol. Apr;71(4):2819-29 (1997), all of which are incorporated herein by reference. A full-length cDNA copy of the Sindbis virus genome is prepared and inserted into the SmaI site of a plasmid derived from pBR322 as described by Lindquist et al., Virology 151:10 (1986). The sequence for the viral coat protein and the adjacent E1 and E2 glycoprotein transmissibility factors are located on the region corresponding to the 26S RNA region. The vector containing the cDNA copy of the Sindbis virus genome is digested with the appropriate restriction enzymes and exonucleases to delete the coding sequence for the structural proteins.

For example, the structural protein coding sequence is removed by partial digestion with BinI, followed by religation to remove a vital portion of the structural gene. Alternatively, the vector is cut at the 3′-end of the viral nucleic acid. The viral DNA is removed by digestion with Bal31 or Micrococcal S1 nuclease up through the start codon of the structural protein sequence. The synthetic DNA sequence containing the sequence of the viral 3′-tail is then ligated to the remaining 5′-end. The deletion of the coding sequence for the structural proteins is confirmed by isolating Sindbis RNA and using it to infect an avian cell culture. The isolated Sindbis RNA is found to be non-infective under natural conditions.

Alternatively only the coding sequence for the coat protein is deleted and the sequence for the E1 and E2 glycoproteins remain in the vector containing the cDNA copy of the Sindbis genome. In this case, the coat protein coding sequence is removed by partial digestion with AfII followed by religation to reattach the 3′-tail of the virus.

A second alternative method for removing only the coat protein sequence is to cut the vector at the 3′-end of the viral nucleic acid. The viral DNA is removed by digestion with Bal31 or Micrococcal S1 nuclease up through the start codon of the coat protein sequence (the same start codon as for the sequence for all the structural proteins). The synthetic DNA sequence containing the sequence of the 3′-tail is then ligated to the remaining 5′-end.

The deletion of the coding sequence for the coat protein is confirmed by isolating Sindbis RNA and using it to infect an avian cell culture. The isolated Sindbis RNA is found to be non-infective under natural conditions.

### Example 25

Preparation of a Non-Transmissible Western Equine Encephalomyelitis Virus Nucleotide Sequence.

Methods for genetic manipulation of Western Equine Encephalomyelitis Virus are described in Garoff et al., Curr. Opin. Biotechnol. 9(5):464-9 (1998) and Weaver et al., J. Virol. 71(1):613-23 (1997), both of which are incorporated herein by reference. A full-length cDNA copy of the Western Equine Encephalomyelitis Virus (WEEV) genome is prepared as described by Hahn et al., Proc. Natl. Acad. Sci. USA 85:5997 (1988). The sequence for the viral coat protein and its adjacent E1 and E2 glycoprotein transmissibility factors are located on the region corresponding to the 26S RNA region. The vector containing the cDNA copy of the WEEV genome is digested with the appropriate restriction enzymes and exonucleases to delete the coding sequence of the coat protein and the E1 and E2 proteins (structural protein coding sequence).

For example, the structural protein coding sequence is removed by partial digestion with NacI, followed by religation to remove a vital portion of the structural protein sequence. Alternatively, the vector is cut at the 3′-end of the structural protein DNA sequence. The viral DNA is removed by digestion with Bal31 or Micrococcal S1 nuclease up through the start codon of the structural protein sequence. The DNA sequence of the viral 3′-tail is then ligated to the remaining 5′-end. The deletion of the coding sequence for the structural proteins is confirmed by isolating WEEV RNA and using it to infect a Vero cell culture. The isolated WEEV RNA is found to be non-infective under natural conditions.

Alternatively, only the coding sequence for the coat protein is deleted and the sequence for the E1 and E2 glycoproteins remain in the vector containing the cDNA copy of the WEEV genome. In this case, the coat protein coding sequence is removed by partial digestion with HgiAI followed by religation to reattach the 3′-tail of the virus.

A second alternative method for removing only the coat protein sequence is to cut the vector at the 3′-end of the viral coat protein sequence. The viral DNA is removed by digestion with Bal31 or Micrococcal S1 nuclease up through the a vital portion of the coat protein sequence. The DNA sequence containing the sequence of the 3′-tail is then ligated to the remaining 5′-end.

The deletion of the coding sequence for the coat protein is confirmed by isolating WEEV RNA and using it to infect a Vero cell culture. The isolated WEEV RNA is found to be non-infective, i.e., biologically contained, under natural conditions.

### Example 26

Preparation of a Non-Infective Simian Virus 40 Nucleotide Sequence.

Methods for genetic manipulation of Simian viruses are described in Piechaczek et al., Nucleic Acids Res. 27(2):426-428 (1999) and Chittenden et al., J. Virol. 65(11):5944-51 (1991), both of which are incorporated herein by reference. A full-length cDNA copy of the Simian virus 40 (SV40) genome is prepared, and inserted into the AccI site of plasmid pCW18 as described by Wychowski et al., J. Virol. 61:3862 (1987). The nucleotide sequence of the viral coat protein VP1 is located between position 1488 and 2574 of the genome. The vector containing the DNA copy of the SV40 genome is digested with the appropriate restriction enzymes and exonucleases to delete the coat protein coding sequence.

For example, the VP1 coat protein coding sequence is removed by partial digestion with BamHI nuclease, and then treated with EcoRI, filled in with Klenow enzyme and recircularized. The deletion of the coding sequence for the coat protein VP1 is confirmed by isolating SV40 RNA and using it to infect simian cell cultures. The isolated SV40 RNA is found to be non-infective, i.e., biologically contained, under natural conditions.

### Example 27

Novel Requirements for Production of Infectious Viral Vector In Vitro Derived RNA Transcripts.

This example demonstrates the production of highly infectious viral vector transcripts containing 5′ nucleotides with reference to the virus vector.

Construction of a library of subgenomic cDNA clones of TMV and BMV has been described in Dawson et al., Proc. Natl. Acad. Sci. USA 83:1832-1836 (1986) and Ahlquist et al., Proc. Natl. Acad. Sci. USA 81:7066-7070 (1984). Nucleotides were added between the transcriptional start site of the promoter for In Vitro transcription, in this case T7, and the start of the cDNA of TMV in order to maximize transcription product yield and possibly obviate the need to cap virus transcripts to insure infectivity. The relevant sequence is the T7 promoter . . . TATAG\hat{ }ATATTTT . . . where the \hat{ } indicates the base preceding is the start site for transcription and the bold letter is the first base of the TMV cDNA. Three approaches were taken: 1) addition of G, GG or GGG between the start site of transcription and the TMV cDNA ( . . . TATAGGTATTT . . . and associated sequences); 2) addition of G and a random base (GN or N2) or a G and two random bases (GNN or N3) between the start site of transcription and the TMV cDNA ( . . . TATAGNTATTT . . . and associated sequences), and the addition of a GT and a single random base between the start site of transcription and the TMV cDNA ( . . . TATAGTNGTATTT . . . and associated sequences). The use of random bases was based on the hypothesis that a particular base may be best suited for an additional nucleotide attached to the cDNA, since it will be complementary to the normal nontemplated base incorporated at the 3′-end of the TMV (−) strand RNA. This allows for more ready mis-initiation and restoration of wild type sequence. The GTN would allow the mimicking of two potential sites for initiation, the added and the native sequence, and facilitate more ready mis-initiation of transcription in vivo to restore the native TMV cDNA sequence. Approaches included cloning GFP expressing TMV vector sequences into vectors containing extra G, GG or GGG bases using standard molecular biology techniques. Likewise, full length PCR of TMV expression clone 1056 was done to add N2, N3 and GTN bases between the T7 promoter and the TMV cDNA. Subsequently, these PCR products were cloned into pUC based vectors. Capped and uncapped transcripts were made In Vitro and inoculated to tobacco protoplasts or Nicotiana benthamiana plants, wild type and 30k expressing transgenics. The results are that an extra G, . . . TATAGGTATTTT . . . , or a GTC, . . . TATAGTCGTATTTT . . . , were found to be well tolerated as additional 5′ nucleotides on the 5′ of TMV vector RNA transcripts and were quite infectious on both plant types and protoplasts as capped or non-capped transcripts. Other sequences may be screened to find other options. Clearly, infectious transcripts may be derived with extra 5′ nucleotides.

Other derivatives based on the putative mechanistic function of the GTN strategy that yielded the GTC functional vector are to use multiple GTN motifs preceeding the 5′ most nt of the virus cDNA or the duplication of larger regions of the 5′-end of the TMV genome. For example: TATA\hat{ }AGTNGTNGTATT . . . or TATA\hat{ }GTNGTNGTNGTNGTATT . . . or TATA\hat{ }GTATTTGTATTT . . . . In this manner the replication mediated repair mechanism may be potentiated by the use of multiple recognition sequences at the 5′-end of transcribed RNA. The replicated progeny may exhibit the results of reversion events that would yield the wild type virus 5′ virus sequence, but may include portions or entire sets of introduced additional base sequences. This strategy can be applied to a range of RNA viruses or RNA viral vectors of various genetic arrangements derived from wild type virus genome. This would require the use of sequences particular to that of the virus used as a vector.

### Example 28

Infectivity of Uncapped Transcripts.

Two TMV-based virus expression vectors were initially used in these studies pBTI 1056 which contains the T7 promoter followed directly by the virus cDNA sequence ( . . . TATAGTATT . . .), and pBTI SBS60-29 which contains the T7 promoter (underlined) followed by an extra guanine residue then the virus cDNA sequence ( . . . TATAGGTATT . . . ). Both expression vectors express the cycle 3 shuffled green fluorescent protein (GFPc3) in localized infection sites and systemically infected tissue of infected plants. Transcriptions of each plasmid were carried out in the absence of cap analogue (uncapped) or in the presence of 8-fold greater concentration of RNA cap analogue than rGTP (capped). Transcriptions were mixed with abrasive and inoculated on expanded older leaves of a wild type Nicotiana benthamiana (Nb) plant and a Nb plant expressing a TMV U1 30k movement protein transgene (Nb 30K). Four days post inoculation (dpi) long wave UV light was used to judge the number of infection sites on the inoculated leaves of the plants. Systemic, noninoculated tissues, were monitored from 4 dpi on for appearance of systemic infection indicating vascular movement of the inoculated virus. Table 1 shows data from one representative experiment.

TABLE 1

| | |
|---|---|
| Local | |
| infection | Systemic |
| sites | Infection |

| Construct | Nb | Nb 30K | Nb | Nb 30K |
|---|---|---|---|---|
| pBTI1056 | | | | |
| Capped | 5 | 6 | yes | yes |
| Uncapped | 0 | 5 | no | yes |
| PBTI SBS60-29 | | | | |
| Capped | 6 | 6 | yes | yes |
| Uncapped | 1 | 5 | yes | yes |

Nicotiana tabacum protoplasts were infected with either capped or uncapped transcriptions (as described above) of pBTI SBS60 which contains the T7 promoter followed directly by the virus cDNA sequence (TATAGTATT . . . ). This expression vector also expresses the GFPc3 gene in infected cells and tissues. Nicotiana tabacum protoplasts were transfected with 1 mcl of each transcriptions. Approximately 36 hours post infection transfected protoplasts were viewed under UV illumination and cells showing GFPc3 expression. Approximately 80% cells transfected with the capped PBTI SBS60 transcripts showed GFP expression while 5% of cells transfected with uncapped transcripts showed GFP expression. These experiments were repeated with higher amounts of uncapped inoculum. In this case a higher proportion of cells, >30% were found to be infected at this time with uncapped transcripts, where >90% of cells infected with greater amounts of capped transcripts were scored infected.

These results indicate that, contrary to the practiced art in scientific literature and in issued patents (Ahlquist et al., U.S. Pat. No. 5,466,788), uncapped transcripts for virus expression vectors are infective on both plants and in plant cells, however with much lower specific infectivity. Therefore, capping is not a prerequisite for establishing an infection of a virus expression vector in plants; capping just increases the efficiency of infection. This reduced efficiency can be overcome, to some extent, by providing excess In Vitro transcription product in an infection reaction for plants or plant cells.

The expression of the 30K movement protein of TMV in transgenic plants also has the unexpected effect of equalizing the relative specific infectivity of uncapped verses capped transcripts. The mechanism behind this effect is not fully understood, but could arise from the RNA binding activity of the movement protein stabilizing the uncapped transcript in infected cells from prereplication cytosolic degradation.

Extra guanine residues located between the T7 promoter and the first base of a virus cDNA lead to increased amount of RNA transcript as predicted by previous work with phage polymerases. These polymerases tend to initiate more efficiently at . . . TATAGG or . . . TATAGGG than . . . TATAG. This has an indirect effect on the relative infectivity of uncapped transcripts in that greater amounts are synthesized per reaction resulting in enhanced infectivity.

Data Concerning Cap Dependent Transcription of pBTI1056 GTN#28.

TMV-based virus expression vector pBTI 1056 GTN#28 which contains the T7 promoter (underlined) followed GTC bases (bold) then the virus cDNA sequence ( . . . TATAGTCGTATT . . . ). This expression vector expresses the cycle 3 shuffled green fluorescent protein (GFPc3) in localized infection sites and systemically infected tissue of infected plants. This vector was transcribed In Vitro in the presence (capped) and absence (uncapped) of cap analogue. Transcriptions were mixed with abrasive and inoculated on expanded older leaves of a wild type Nicotiana benthamiana (Nb) plant and a Nb plant expressing a TMV U1 30k movement protein transgene (Nb 30K). Four days post inoculation (dpi) long wave UV light was used to judge the number of infection sites on the inoculated leaves of the plants. Systemic, non-inoculated tissues, were monitored from 4 dpi on for appearance of systemic infection indicating vascular movement of the inoculated virus. Table 2 shows data from two representative experiments at 11 dpi.

TABLE 2

| | |
|---|---|
| Local | |
| infection | Systemic |
| sites | Infection |

| Construct | Nb | Nb 30K | Nb | Nb |
|---|---|---|---|---|
| 30K | | | | |
| Experiment 1 | | | | |
| pBTI1056 GTN#28 | | | | |
| Capped | 18 | 25 | yes | yes |
| Uncapped | 2 | 4 | yes | yes |
| Experiment 2 | | | | |
| pBTI1056 GTN#28 | | | | |
| Capped | 8 | 12 | yes | yes |
| Uncapped | 3 | 7 | yes | yes |

These data further support the claims concerning the utility of uncapped transcripts to initiate infections by plant virus expression vectors and further demonstrates that the introduction of extra, non-viral nucleotides at the 5′-end of in vitro transcripts does not preclude infectivity of uncapped transcripts.

### Example 29

Methods for Inhibiting Endogenous Proteolytic Activity in Plants in vivo.

Elicitor recognition and the response cascades occurring in plants form an essential link between the environmental stress and plant survival responses. Many products are induced following induction by environmental stimuli or pathogen infection, which include, but are not limited to, proteases, protease inhibitors, alkaloids and other metabolites. Glazebrook, et al., Annu. Rev. Gen. 31:547-569 (1997); Grahm, et al., J. Biol. Chem. 260:6555-6560 (1985); and Ryan, et al., Ann. Rev. Cell Dev. Biol. 14:1-17 (1998), all incorporated herein by reference. The components of the recognition and response pathways are poorly understood, yet have tremendous practical value for input traits in genetically improved crops. Traditional methods of mutagenesis or biochemistry are leading to slow and incremental advances in our understanding. However, if these pathways are to be elucidated, understood and exploited, more rapid discovery methods must be brought to bear on the problem. Virus expression vectors capable of either overexpressing gene products or suppressing the expression of particular endogenous host genes provide a unique tool to discover the nature of the genes whose products contribute to the response pathways.

This example describes methods for inhibiting endogenous plant proteases which interfere with the expression and purification of recombinant proteins in plants. In particular, this example shows methods for inhibiting proteolytic activity in planta which is responsible for the degradation of a viral vector-expressed recombinant protein. These methods are also applicable to the protection of recombinant proteins expressed via a stable transformation system or endogenous plant proteins. Viral vectors have been constructed to include an N-terminal signal peptide sequence. This sequence directs the recombinant protein through the secretory pathway to the cell surface and ultimately accumulating in the plant intercellular fluid (IF) (Kermode, Critical Reviews in Plant Sciences 15(4):285-423 (1996), incorporated herein by reference). In some instances, the target protein was cleaved aberrantly in vivo. Three examples include a mammalian growth hormone and single chain antibody and an avian interferon. In vivo residence time in the IF led to the accumulation of the cleavage product(s) as detected by immunoblotting. Cleavage was either complete in vivo or continued In Vitro following IF extraction (Co-pending U.S. patent application Ser. No. 09/037,751, incorporated herein by reference). Quantitation of western blots using UVP Gelbase/Gelblot-Pro software revealed as much as 40-50% of the expressed protein was cleaved.

We designed In Vitro experiments to inhibit the plant proteolytic activity. When we added protease inhibitors to an isolated IF fraction In Vitro, we were able to inhibit further degradation of our recombinant protein. In addition, when we treated an IF fraction from an unrelated virally infected plant with protease inhibitors and incubated that with a known susceptible protein, we completely inhibited the protease and protected the protein from degradation.

Following the observation that the cleavage was occurring in vivo by a plant protease that could be inhibited by proteinase inhibitors, we designed experiments to inhibit this activity in planta. Three possible methods to inhibit the protease are as follows:

1. Recombinant Expression of a Proteinase Inhibitor:

The activity of the plant protease may be inhibited by the recombinant expression of a plant proteinase inhibitor secreted to the IF based on the following results:
(1) We cloned a tomato proteinase inhibitor gene (Wingate, et al., J. Biol. Chem. 264:17734-17738 (1989), incorporated herein by reference) into our viral vector. We verified that the expression of the recombinant inhibitor protein was in the IF fraction by western detection. Virally-expressed proteinase inhibitor protected our recombinant (E. coli-derived) mammalian growth hormone protein standard that was known to be susceptible to the plant protease in an In Vitro assay;
(2) Virally-expressed proteinase inhibitor specifically inhibited an IF-localized protease in vivo as per detection on Zymogram gelatin Tris-glycine gels; and
(3) Co-inoculation of the virus vector proteinase inhibitor construct and the viral vector mammalian growth hormone construct resulted in the expression of both proteins in systemic leaves and partial protection of the growth hormone in the IF.

Another possible approach is to combine transgenic plants and virally-expressed proteins. One could either inoculate the virus vector proteinase inhibitor construct on transgenic plants expressing a target protein or make a proteinase inhibitor transgenic plant and inoculate with a viral vector construct expressing the target sequence.

2. Induction of Endogenous Proteinase Inhibitors:

One could also induce the endogenous production of plant proteinase inhibitors using an elicitor. For example, jasmonic acid (JA) is produced as part of a general plant defense mechanism and is known to induce specific proteinase inhibitors (Lightner et al., J Mol Gen Genet. 241:595-601 (1993), incorporated herein by reference). Exogenous application of JA as been used to induce a plant defense response in Nicotiana attenuata to against herbivore attack (Baldwin, PNAS, 95(14):8113-8118 (1998), incorporated herein by reference). To protect against specific endogenous proteolysis of a recombinant protein, one could treat the plant material with JA to induce the synthesis of the proteinase inhibitor and then inoculate with a viral vector construct expressing the target sequence.

The desired phenotype in host plants used for gene discovery program using virus expression vectors is reduced proteolytic activities in the cytosol, secretory pathway or apoplast so to increase the half-life of virally produced proteins. This will allow virally expressed proteins to exert their influence on plant biochemistry, development and growth optimally. Rapid or premature degradation may reduce the amount of the expressed protein below the necessary threshold to exert a measurable effect. Transgenic expression of protease inhibitors, such as those induced by the systemin pathway (Ryan, et al., Ann. Rev. Cell Dev. Biol. 14:1-17 (1998)), will provide a continuous source of inhibitor to slow particular degradation processes. Conversely, as outlined in the example above, treating virus vector infected plants with JA will induce the response pathways and result in the expression of various inhibitors in infected/treated plants. In both ways, by specific protease inhibitor expression or by induction of response cascade, the half-lives of many proteins, whose presence is requisite for detecting the novel finctions of gene products, are increased.

### Example 30

Selection of Optimized RNA and Protein Activities by Use of Virus Vectors to Express Libraries of Sequence Variants Generated by Means of In Vitro Mutagenenisis and/or Recombination.

DNA shuffling is a process for recursive mutation and In Vitro recombination, performed by random fractionation and re-assembly of a gene of interest to generate a pool of related, yet not identical, gene sequences. Stemmer et al., U.S. Pat. Nos. 5,830,721 and 5,811,238, incorporated herein by reference. Fractionation occurs through the treatment of DNA sequences with limiting amounts of nuclease and re-assembly typically requires two steps, first primerless PCR to re-align fragments based on local homology and then primer driven PCR to recover full length assembled fragments. The advantages of this approach are many: (1) gene or sequence function can be optimized or improved without first determining the sites within the sequence that require alteration; (2) several generations of “improved” sequences can be generated, given proper selection, in time frame unattainable by natural circumstances; (3) mutations of every sort are randomly dispersed throughout the gene sequence allowing a “saturation” approach to determine the genetic potential of a given sequence. Crameri et al., Nature Biotech. 14:315 (1996); Crameri et al., Nature Biotech. 15:436 (1997); Zhang et al., Proc. Natl. Acad. Sci. USA 94:4504 (1997); Zhao and Arnold, Proc. Natl. Acad. Sci. USA 94:7997 (1997).

DNA shuffling has been successfully applied to prokaryotic or cell-based systems to select sequences of desired protein activities. However, the ability to introduce shuffled sequences throughout an organism in a rapid and high throughput manner necessary to harness the full potential of this technology has not been demonstrated. In this example, we describe the use of plant virus expression vectors to bear populations of shuffled DNA sequences and were applied to plant hosts and those sequences with desired properties were selected and further characterized. The properties conferred by the selected shuffled sequences were demonstrated to be inherited by progeny viruses.

Two aspects that must be continually improved in virus expression vectors are: 1) their ability to move in a facile manner both locally and systemically in plants, and 2) the need for greater levels of foreign gene expression. Both of these finctions can potentially be affected by modifications to the 30 kDa ORF. Functions within the 30 kDa coding region include the movement protein (MP), the virus origin of virion assembly and the subgenomic promoter used for coat protein synthesis. This is the promoter used for expression of foreign gene sequences in most tobamovirus vectors. It has been demonstrated that natural variation in viral populations can be the substrates for selection of improved characters in viral vectors can lead to dramatic improvements in their performance. This work further showed that single or multiple amino acid substitutions in the 30 kDa ORF can significantly effect the movement properties of virus vectors. Viruses function genomically, as an integrated whole of RNA and protein sequences, suggesting that either individual elements, such as the 30 kDa ORF, or the entire plant virus genomes could be subjected to shuffling so to improve plant virus vector performance. Obvious following the application of shuffling in this context is the use of plant virus vectors to house shuffled foreign gene populations which, following inoculation onto plants, gene products with optimized activities can be selected. Plant virus vectors are the ultimate tool for shuttling genes into plants for selection of optimized activities. No other tool, transient or stable expression methods, can match the ability of plant virus vectors to develop optimized genes for plant activities.

Experiments to demonstrate the ability of plant viruses to house libraries of sequence variants focused on optimizing the coding region for the 30 kDa movement protein from TMV U1 for movement properties in Nicotiana tabacum and subgenomic promoter activity responsible for coat protein mRNA production. The base expression vector, p30B GFP, was used as a tool to be modified as desired for a shuffling vector. p30B GFP vector is the TMV U1 infectious cDNA (bases 1-5756) containing the 5′ NTR, replicase genes (126 and 183 kDa proteins), movement protein gene with associated subgenomic promoter and an RNA leader derived from the U1 coat protein gene. Following the RNA leader is a unique PacI site and the green fluorescent protein (GFP) gene. Following a unique XhoI site, the clone continues with a portion of the TMV U1 3′ NTR followed by a subgenomic promoter, coat protein gene and 3′ NTR from TMV U5 strain.

The first stage of the project required the construction of a vector into which shuffled DNA fragments could be reintroduced. The polymerase chain reaction (PCR) was used to amplify a DNA fragment from the TMV vector p30B comprising the T7 promoter, 5′ non-translated region (NTR), and the reading frames for the 126 and 183 kDa replicase proteins. The 5′ primer covered the T7 promoter and initial bases of the TMV genome while the second primer modified the context surrounding the start codon for the 30 kDa MP of TMV. This allowed DNA fragments to be ligated into the modified vector, designated 30B GFP d30K, as AvrII, PacI restriction endonuclease digested fragments.

```
Native TMV 183/30 kDa junction and 30k/GFP junction
183 kDa ORF
AGT TTG TTT ATA GAT GGC TCT AGT TGT TAA AGG AAA A . . . GAT TCG TTT TAA (cont.)
S L F I D G S S C *
                                    M A L V V K G K . . . D S F *
                                  30kDa ORF
ATAgaTCTTACAGTATCACTACTCCATCTCAGTTCGTGTTCTTGTCATTAATTAA ATG . . .
                                                                                              PacI GFP ORF
Modified TMV 183/30 kDa/GFP junction (without 30 kDa gene): p30B d30k ANP
183 kDa ORF
AGT TTG TTT ATA GAc GGC TCT AGT TGT TAA g CCTAGG A GCCGGC TTAATTAA ATG . . .
GFP ORF
S L F I D G S S C * AvrII NgoMI PacI
Modified TMV 183/30 kDa junction and 30k/GFP junction (with 30 kDa gene present)
183 kDa ORF
AGT TTG TTT ATA GAT GGC TCT AGT TGT TAA g _ ATG GCT CTA GTT GTT AAA GGA
AAA . . .
S L F I D G S S C * AvrII
                                                                                M A L V V K G K . . .
. . . GGTTTTAAATAgaTCTTACAGTATCACTACTCCATCTCAGTTCGTGTTCTTGTCATTAATTAA ATG . . .
                                                                                                                        PacI GFP ORF
```

This modification allowed the ready insertion of modified 30 kDa gene fragments into a virus vector and have them expressed in plant cells, tissues or systemically. The wild type GFP ORF is the reporter gene since the visual level of fluorescence as observed under long wave UV light correlates directly with levels of GFP protein present in plant tissues. This has been demonstrated by looking at different virus vectors expressing GFP, each having different strength subgenomic promoters, that were infected in plants and GFP levels determined by UV fluorescence and Western blotting using anti-GFP antibodies.

The procedure for shuffling of the 30 kDa gene is similar to that described by Crameri et al., Nature Biotech. 15:436 (1997), and contained the following steps. The 30 kDa gene fragment also containing the coat protein RNA leader was amplified from tobamovirus expression vectors using primers: TMVU1 30K 5′A (5′-GGCCCTAGGATGGCTCTAGTTGTTAAAGG-3′) (SEQ ID NO: 48) and 3-5′ Pac primer (5′-GTTCTTCTCCTTTGCTAGCCATTTAATTAATGAC-3′) (SEQ ID NO: 49). The PCR DNA product was gel isolated and then incompletely digested with DNaseI. DNA fragments of 500 bp or smaller were isolated by using DEAE blotting paper technique and then eluted. Purified DNA fragments were mixed together with taq DNA polymerase and allowed to “reassemble” for 40 cycles. “Reassembly” reaction was assayed by gel electrophoresis for DNA bands of approximately 800-850 bp. Approximately 1 mcl of the “reassembly” reaction was then subjected to PCR using primers TMV U1 30K 5′A and 3-5′ Pac that hybridize to terminal DNA ends of reassembled fragments. The reassembled fragments will be gel isolated and digested with restriction enzymes AvrII and PacI (sites present in the terminal primers) to allow for facile cloning back into the p30B d30k ANP digested with AvrII and PacI.

Ligations of shuffled genes into p30B d30k ANP resulted in pooled libraries of sequences containing 100 to 50,000 members in five separate experiments. Pooled virus vectors with libraries of variant 30 kDa coding regions were transcribed with T7 RNA polymerase and then inoculated by standard PEG transfection into 0.5×10⁶ Nicotiana tabacum protoplasts per sample. Inspection of cells 24 hours post inoculation revealed varied intensities of GFP fluorescence in individual cells indicating possible different levels of GFP accumulation and possible effects in the subgenomic promoter activity as desired. Cells were incubated for 48 hours post inoculation, harvested by centrifugation and then lysed using freeze/thaw and grinding with a mortar and pestle. The virions that accumulated in protoplasts were released by the grinding.

The protoplast extracts were then inoculated on leaves of wild type and transgenic Nicotiana tabacum c.v. MD609 expressing the TMV U1 30 kDa movement protein. Three to five days post inoculation localized infection sites were observed expressing GFP. A variety of intensities of GFP fluorescence were observed varying from that observed with the wild type GFP gene to much duller to very bright, as observed from the viral expression of the shuffled GFP gene of Crameri et al., Nature Biotech. (1996) (GFPc3). The occurrence of viruses expressing enhanced GFP fluorescence varied between libraries tested from 1/200 to 1/50 infection foci depending on libraries tested. These local infection sites with enhanced GFP fluorescence were excised from the leaves and inoculated on Nicotiana benthamiana plants. The bright local infection variants were then purified on the inoculated leaves of these plants from contaminating viruses expressing less GFP protein. These viruses expressing brighter GFP proteins were found to express larger amounts of GFP protein in systemic tissues than the starting p30B GFP virus. Sequencing and genetic studies indicated that no mutations accumulated in the GFP genes and that the effects were due to mutations in the TMV U1 30 kDa ORF that up regulated the subgenomic promoter. The accumulation of GFP in the shuffled variants with brighter GFP phenotype was 3.4 fold greater than that produced by p30B GFP as measured by quantitative Western blotting of plant extracts using an anti-GFP sera. These data demonstrated that shuffling could be used to enhance the cis-acting functions of RNA sequences and that plant RNA virus expression vectors are effective tools to shuttle large diversity of sequence variants in whole plants and plant cells.

The protoplast extracts isolated from transfections with virus libraries were inoculated on one half of wild type Nicotiana tabacum c.v. MD609 and Nicotiana benthamiana leaves. To the other leaf half, virus derived from p30B GFP was inoculated. Some infection sites resulting from infection of viruses containing shuffled 30 kDa ORFs grew more rapidly than those of the average from p30B GFP. These events occurred at a frequency of 1/100 to 1/500 infection foci depending on the virus library analyzed. These more rapidly growing infection foci were excised and inoculated on young Nicotiana tabacum c.v. MD609 plants. As a control, p30B GFP was inoculated on similar sized and aged plants. The p30B GFP vector does not move systemically on tobacco plants. However, some shuffled 30 kDa ORF variant vectors, that were identified as rapidly growing local infection sites, were able to move systemically on tobacco plants. The movement was primarily on phloem source tissue and were localized to veins and circular spots in green lamina. This movement ability was reproducible in multiple inoculations of these individual virus variants. Sequence analysis of the viruses containing shuffled 30 kDa ORFs capable of systemic movement on Nicotiana tabacum plants demonstrated that localized amino acid substitutions were present and responsible for altered movement phenotype.

Further recursive shuffling of the top 5-10% of GFP expressing vectors or those that demonstrated an enhanced ability to invade systemic tissues of tobacco could be carried out to meld synergistic mutations to lead to greater gains in expression or virus movement. Likewise, the 30 kDa ORFs that contain the most potent subgenomic promoters and most enabled movement activities in tobacco could be shuffled together so to bring both sets of properties into the same 30 kDa ORF. It is also apparent from these data that by testing virus expression vectors containing libraries of these shuffled variants, one can select the variant with the protein or RNA activity that one desires. The phenotypes that can be assayed are protein activity in planta, as with the movement activities of the 30 kDa protein, enzyme activities in planta or in plant extracts or other surrogate features such as substrate or product accumulation. These data demonstrate the power of virus expression vectors to be effective tools for shuttling sequence variants into plants and allow the selection of genes encoding the desired altered property. This tool allows one to mine the hidden activities, enhance the isolated activities of enzymes or eliminate allosteric inhibition of enzyme activities. This could be applied to any plant gene or genes from other sources to optimize the activities desired for agronomic, pharmaceutical or developmental effects caused by altered genes.

### Example 31

Composite Cloning to Facilitate Cloning of Libraries in Virus Vectors and/or their Introduction into Host Cells for Expression of Sequences.

Virus vector clones could be integrated into lambda phage or cosmid clones to facilitate library construction, clone representation, elimination of cell based amplification by direct transcription and archiving of individual clones. Likewise, cis-acting elements allowing for expression in plant cells or integration into plant DNA could be included into such plasmids to facilitate inoculation of DNA for direct expression, obviating the need for transcription of vector cDNA, or construction of dedicated plant transformation vectors.

Virus vectors are tools housing libraries of sequences that can be screened for novel gene discovery. However libraries are often first constructed in plasmid or phage shuttle vectors before excising and introduction into virus vectors. Likewise, sequences can be screened in hosts using virus vectors, but must be subcloned into appropriate eukaryotic expression vectors before the trait identified in the vector transfected host will become a stable trait in the host by gene integration. Additional hurdles to overcome are: (1) construction of libraries to most efficiently represent the clones in a cDNA library, (2) obtaining maximal transfection efficiency into bacterial hosts (if used), and (3) archiving DNA samples without the need for transfection into bacteria and transcription of ligated DNA. The integration of a virus vector into a cosmid clone, or lambda phage itself, (both termed phagmids here) could allow a multi-purpose vector to be generated to be both the repository of primary generated library sequences, source for ligation transcriptions, high efficiency bacterial transfection and direct expression in higher eukaryotic hosts. Using normal cloning procedures, the 5′ half of the virus vector to be inserted into one arm of a phagmid DNA clone with a non symmetrical restriction (such as BstXI: CCANNNNNNTGG) containing a unique sticky sequence (the N's). The 3′ part of the vector will be inserted into another arm with a non-symmetrical restriction (such as BstXI: CCANNNNNTGG) containing a second unique sticky sequence (the N's). The vector would be split at the determined restriction site (e.g. BstXI) within the site for foreign sequence expression in the virus vector. The 5′-end of the virus cDNA would be appropriately fused to a promoter for In Vitro transcription (e.g. T7) or for in vivo expression (e.g. an appropriate higher eukaryotic RNA polymerase promoter). The 3′-end of the virus cDNA would terminate with a ribozyme for In Vitro cleavage and/or a 3′ terminator from a gene from host organism to lead to in vivo termination of transcription. Left and right T-DNA borders that promote the integration of sequences in between into plant genomic DNA, could flank the promoter and terminator sequences. At the terminus of each arm would be cos sequences to allow complete regeneration of the phagmid upon ligation in the presence of foreign library DNA containing the two unique sticky sequences at each respective termini. These library DNA fragments could be generated by PCR amplification using determined restriction sites (e.g., BstXI) to generate unique sticky ends complementary to those in the phagmid-vector arms integrated in the PCR primers. The 5′ and 3′ primers would each have unique recognition sequences in the BstXI restriction site (the N's) that would match the sticky sites on the respective sides of the virus vector. The sites could be switched on a second set of PCR primers to allow the amplification of DNA to be ligated into the phagmid-viral vector arms in the “sense” and “anti-sense” orientation. These constructions would allow for efficient In Vitro ligation and use of crude ligation mix as template for E. coli transformation, plant transformation, In Vitro lambda packaging to 10⁹ pfu/mcg or In Vitro transcription. In this manner, the vector and flexibility for its screening could be maximized. These tools we can directly build complex libraries into and simultaneously be the enabling tool for analysis.

### Example 32

Improvement of Host Plant Performance with a Viral Expression System via Interspecific Hybridization.

The goal of this example is to improve the host plant by introducing foreign genetic material via interspecific hybridization. Host plant species vary in their ability to support expression of a sequence inserted into a plant viral vector. Some species support expression to a high specific activity, such as Nicotiana benthamiana, but have relatively low biomass. Other species, such as N. tabacum, have high biomass and/or other desirable properties for growth in the field, but have a relatively low specific activity of the expressed sequence. In this example, the desirable properties of two or more species are combined by making an interspecific hybrid by standard methods. After chromosome doubling to restore fertility, the primary hybrid may have suitable properties, or it may be desirable to backcross toward either parent selecting or screening at each generation for the desired property(ies) of the non-recurrent parent, for example, introgress the superior biomass of N. tabacum into N. benthamiana, or introgress the superior viral vector performance of N. benthamiana into N. tabacum, among others. A viral vector expressing the green fluorescent protein (GFP) is one example of a useful tool for screening the level of systemic expression in candidate hybrid plants.

Many hybrids are possible, especially within the genus Nicotiana. For example, we have hybrids between N. benthamiana and N. tabacum. N. benthamiana and N. clevelandii, N. benthamiana and N. excelsior, N. benthamiana and N. africana, N. clevelandii and N. africana, N. umbratica and N. africana, N. umbratica and N. otophora, and N. bigelovii and N. excelsior. In addition, hybrids with more than two parents are possible. For example, we have N. benthamiana/tabacumlafricana and N. benthamiana/clevelandii/tabacum.

### Example 33

Libraries of Heterologous Nucleic Acid Sequences in DHSPES Constructs Generated in a Restriction-Endonuclease-Free and Cell-Free Manner.

The goal of this example is to generate libraries of DHSPES constructs containing heterologous sequences while avoiding the potential problems associated with the use of restriction enzymes for preparation of the inserted nucleic acids and with passage of the resultant constructions through E. coli.

Normally, DNA fragments are generated by restriction endonuclease treatment and ligated into a DHSPES vector with compatible termini. However, when a complex population of DNA molecules, such as that found in a cDNA library, is used as starting material and a given restriction endonuclease is used to treat the insert DNA to render the appropriate termini for ligation to the cloning vector, the recognition sequence for that enzyme will occur with a certain frequency within the population, rendering the molecule bearing that sequence truncated after digestion.

Passage of certain plasmid-based viral clones through E. coli has been observed to result in instability of the plasmid a certain proportion of the time. The cause of this instability is unclear, but may be related to insert size, sequence or to toxicity resulting from expression of the gene from cryptic promoter sequences present in the DHSPES viral sequences.

In order to avoid the above-mentioned problems, libraries of DHSPES constructs harboring cDNA molecules in a restriction endonuclease-free and E. coli-free manner are constructed. Such a system will permit the inclusion into DHSPES constructs of molecules that harbor inconvenient internal restriction sites. This method of “cell-free cloning” will also allow us to obtain DHSPES-derived viruses containing genes that are not well tolerated by E. coli in traditional cloning approaches.

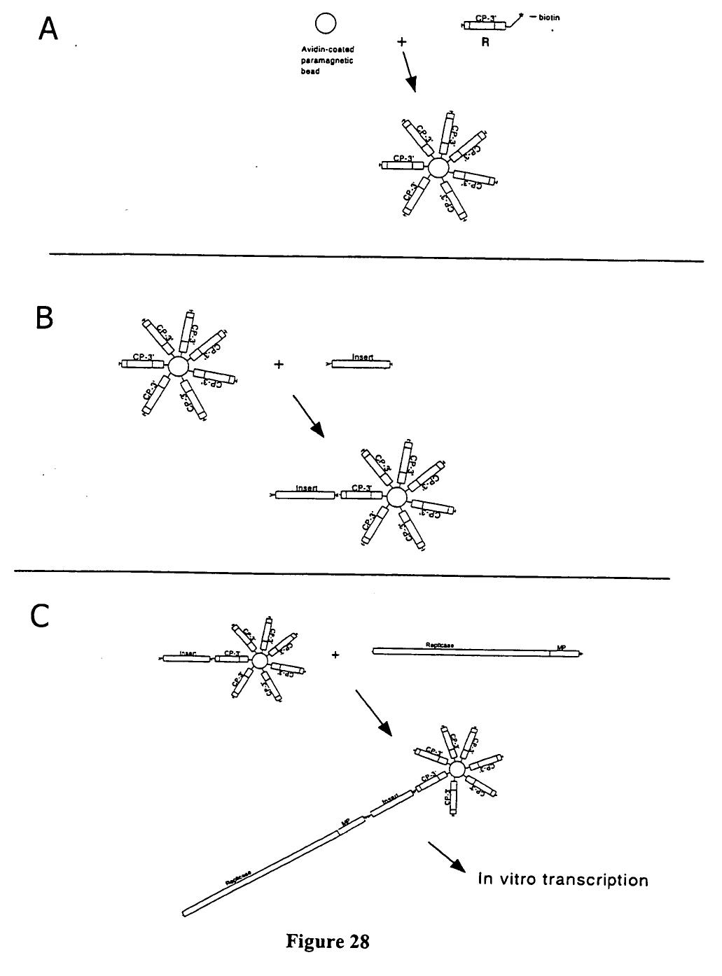

In essence, cell-free cloning will entail the In Vitro assembly of partial viral sequences with a DNA fragment into a configuration that that will yield infectious viral RNA molecules upon In Vitro transcription. In one system, the viral sequences are divided into two “arms”; the left arm and the right arm. The left arm encodes a T7 RNA polymerase promoter followed by viral sequences encoding replicase followed by the gene encoding movement protein and the subgenomic promoter that controls expression of the desired gene. The right arm will contain sequences of the viral genome that encode the viral coat protein and the sequences that control its expression, the viral 3′ untranslated region, and a ribozyme sequence for generating the desired 3′ terminus on the transcribed molecules. A schematic diagram for cell free cloning is shown in FIG. 28.

The left arm and right arm will each have separate asymmetric (non-palindromic, thus self-incompatible) overhangs that will permit the two arms to be brought together by an intervening insert that is derived either from PCR product, cDNA reaction, or elsewhere. The insert will have termini that are compatible with both the left and right arms. The termini of these molecules are such that ligation of left and right arms to insert will ensure assembly into the proper configuration to yield infectious viral transcripts. The sequence contained in the insert will then be in the correct orientation and genomic position to permit its expression from the virus in plant cells.

Specifically, the right arm will be synthesized by PCR and will have a biotin group incorporated into the reverse (3′) primer. The resulting biotinylated PCR product representing the right arm will then be immobilized upon streptavidin paramagnetic beads. Treatment of the DNA with T4 DNA polymerase and a single dNTP (in the present case, dGTP) will give a 5′ overhang as a result of the exonuclease activity of the polymerase. The insert DNA, being PCR product, restriction fragment, or cDNA will be treated with T4 DNA polymerase with a single dNTP to generate 5′ overhangs on its termini; the 3′ of which is compatible with the 5′ of the right arm. The 5′ terminus of the insert DNA will be compatible with the left arm 3′ terminus that had been generated similarly.

The ligation reactions in the assembly of the virus on the paramagnetic beads will be carried out sequentially, with the insert being ligated to the immobilized right arm first, followed by washing of the bead complex and then ligation of the left arm. Following the subsequent wash, In Vitro transcription will be carried out to generate infectious RNA transcripts.

In this cell-free manner, replication-competent viruses expressing the GFP gene were constructed. Using PCR, a biotinylated right arm was prepared. Following immobilization on avidincoated paramagnetic beads and treatment with T4 DNA polymerase and a single nucleotide (dGTP) to generate the appropriate 5′ overhang, the right arm was ligated to a PCR product encoding the GFP gene that had been treated with T4 DNA polymerase and dCTP to render a compatible 5′ overhang. A DNA fragment comprising the left arm of the virus was then ligated to the resulting DNA-bead complex to generate a full-length virus clone that was subsequently used as template for In Vitro transcription. After each step of enzymatic manipulation of the magnetic bead-bound DNA, DNA-bead complexes were washed by sedimenting them in a magnetic field and resuspending them in the appropriate buffer. In addition, after each manipulation, aliquots were taken for analysis to confirm that the desired reaction had occurred. The infectious RNA products of the transcription reaction were introduced into protoplasts of tobacco cell suspension cultures. At 12-18 hours after protoplast infection, fluorescence emitted by the GFP encoded by the virus clone was observed in a majority of the cells confirming that the RNA transcript derived from the DNA-bead complexes was infectious, and hence, that the sequentially assembled virus-encoding DNA molecules had been assembled in the desired configuration so as to permit virus replication and expression of the inserted foreign gene sequences.

### Example 34

Use of Undefined Sequences to Increase the Genetic Stability of Foreign Genes in Virus Expression Vectors.

Insertion of foreign gene sequences into virus expression vectors can result in arrangements of sequences that interfere with normal virus function and thereby, establish a selection landscape that favors the genetic deletion of the foreign sequence. Such events are adverse to the use of such expression vectors to stably express gene sequences systemically in plants. A method that would allow sequences to be identified that may “insulate” functional virus sequences from the potential adverse effects of insertion of foreign gene sequences would greatly augment the expression potential of virus expression vectors. In addition, identification of such “insulating” sequences that simultaneously enhanced the translation of the foreign gene product or the stability of the mRNA encoding the foreign gene would be quite helpful. The example below demonstrates how libraries of random sequences can be introduced into virus vectors flanking foreign gene sequences. Upon. analysis, a subset of introduced sequences allowed a foreign gene sequence that was previously prone to genetic deletion to remain stabily in the virus vectors upon serial passage. The use of undefined sequences to enhance the stability of foreign gene sequences can be extrapolated to the use of undefined sequences to enhance the translation of foreign genes and the stability of coding mRNAs by those skilled in the art.

The genetic stability of the human growth hormone gene (hGH) or an Ubiquitin fusion to hGH (Ubiq hGH) in the tobamovirus expression vector p30B is rather poor, such that no stable virus preparations could not be made to serially passage infection onto plants and detect the expression of hGH recombinant protein. The site of gene insertion is following a PacI site (underlined) in the virus vector. This sequence is known as a leader sequence and has been derived from the native leader and coding region from the native TMV U1 coat protein gene. In this leader, the normal coat protein ATG has been mutated to a Aga sequence (underlined in GTTTTAAATAgaTCTTACAGTATCACTACTCCATCTCAGTTCGTGTTCTTGTCA TTAATTAA ATG . . . (hGH GENE)). A particular subset of this leader sequence (TCTTACAGTATCACTACTCCATCTCAGTTCGTGTTCTTGTCA) has been known to increase genetic stability and gene expression when compared with virus construct lacking the leader sequence. The start site of subgenomic RNA synthesis is found at the GTTTT . . . An oligonucleotide RL-1 (GTTTTAAATAGATCTTAC N(20)TTAATTAAGGCC ) was used with a primer homologous to the NcoI/ApaI region of the TMV genome to amplify a portion of the TMV movement protein. The population of sequences were cloned into the Apal and PacI sites of the p30B hGH vector. Vectors containing the undefined sequences leading the hGH genes were transcribed and inoculated onto Nicotiana benthamiana plants. 14 days post inoculation, systemic leaves were ground and the plant extracts were inoculated onto a second set of plants. Following the onset of virus symptoms in the second set of plants, Western blot analysis was used to detect if hGH or Ubiq-hGH fusions were present in the serially inocuated plants. Several variants containing novel sequences in the non-translated leader sequence were identified that were associated with viruses that were genetically stable and allowed successful passage of hGH expression on plants inoculated with serially passaged virus. Whereas the parental controls, p30B hGH and p30B Ubiq-hGH, did not. Viruses derived from undefined sequence library, p30B hGH virus #2 and #5, were shown to genetically stable upon virion passage and likewise, p30Ubiq hGH #6 showed expression of the Ubiq-hGH expression upon serial virion passage. Again, this property was never observed in each of the starting viruses p30B hGH and p30Ubiq hGH. The sequence surrounding the leader was determined and compared with that of the control virus vectors.

| | |
|---|---|
| p30B #5 HGH | GTTTTAAATAGATCTTAC--TATAACATGAATAGTCATCG |
| p30B #5 HGH | GTTTTAAATAGATCTTAG--TATACCATGAATTAGTACCG |
| p30B #6 UbiqHGH | GTTTTAAATAGATCTTAC--ACTCGGTTGAGATAAAACTAAACTA |
| p30B #2 HGH | GTTTTAAATAGATCTTAC--TCCGACGTATAGTCACGACG |
| p30B HGH | GTTTTAAATAGATCTTAC--AGTATCACTACTCCATCTCAGTTCGTGTTCT |
| p30BUbiqHGH | GTTTTAAATAGATCTTAC--AGTATCACTACTCCATCTCAGTTCGTGTTCT |
| | *********************** |
| p30B #5 HGH | -----TTAATTAAAATGGGA--- |
| p30B #5 HGH | -----TTATTTAAAATGGGAAAAATGGCTTCTCTATTTGCCACATTTTTA |
| p30B #6 UbiqHGH | -----TTAATTAAAATGGGAAAAATGGCTCTCTTATTGGCCCCATTTTTA |
| p30B #2 HGH | -----TTAATTAAAAATGCAGATTTTCGTCAAGACTTTGACCGGG |
| p30B HGH | TGTCATTAATTAAAATGGGAAAAATGGCTTCTCTATTTGCCACATTTTTA |
| p30B UbiqHGH | TGTCATTAATTAAAATGCAGATTTTCGTCAAGACTTTGACCGGT |
| | **************** |

*indicates sequences that are identical in all viruses.
--indicates end of defined primer and start of N(20) region of the oligonucleotide that was introduced during PCR amplification.

The result was that undefined leader constructs transcribed were passageable as virus, while the parental 30B vectors with native leaders were not. The nature of the random leaders indicates that each are unique and that multiple solutions are readily available to solve RNA based stability problems. Likewise, such random sequence introductions could also increase the translational efficiency.

In order to select for undefined sequences that may increase the translational efficiency of foreign genes or increases the stability of the mRNA encoding the foreign gene derived from a virus expression vector, a selectable marker could be used to discover which of the undefined sequences yield the desired function. The amount of the GFP protein correlates with the level of fluorescence seen under long wave UV light and the amount of herbicide resistance gene product correlates with survival of plant cells or plants upon treatment with the herbicide. Therefore introduction of undefined sequences surrounding the GFP or herbicide resistance genes and then screening for individual viruses that either express the greatest level of fluorescence or cells that survive the highest amount of herbicide. In this manner the cells with the viruses with the highest foreign gene activity would be then purified and characterized by sequencing and more thorough analysis such as Northern and Western blotting to access the stability of the mRNA and the abundance of the foreign gene of interest.

### Example 35

Method for Using Reporter Genes Fused to Regulated or Constitutive Promoters as a Surrogate Marker for Identifying Genes Impacting Gene Regulation.

In this example we will show 1) a method to construct transgenic hosts expressing a reporter gene under the control of various promoter types; 2) means to use such hosts to identify genes from libraries expressed in virus expression vectors that alter gene regulation.

The initial construction of the reporter gene expression cassette will require identification of the appropriate reporter gene, which could include GFP (fluorescent in live plants under long wave UV light), GUS (fluorescent and color-based assay in detected tissue), herbicide resistance genes (live or death phenotype upon treatment with herbicide) or other scoreable gene products known to the art. Promoter sequences can express RNA in constitutive or induced conditions. An example of a regulated promoter would be that of tomato or potato protease inhibitor type I gene (Graham, et al., J. Biol. Chem. 260:6555-6560 (1985)). These promoters are up regulated in the presence of jasmonic acid or herbivore damage to plant tissues. Constitutive promoters are readily identifiable from anyone skilled in the art inspecting the relevant literature. Such combinations of inducible or constitutive promoters using appropriate reporter genes would be integrated into binary plant transformation vectors, transformed into Agrobacterium and transformed into Nicotiana benthamiana leaf disks. Upon identification of the appropriate gene construct in regenerated tissues, the primary transformants would be selfed to obtain the first stable line of plants for assay.

Libraries of cDNAs, full-length for gene overexpression or gene fragments for sense or anti-sense based gene suppression, would be ligated into virus expression vectors by normal molecular biology techniques. These libraries would be prepared for inoculation by the methods described in this patent application. Once inoculated, hosts with inducible promoters fused to reporter genes, maintained in uninduced state, would be monitored for aberrant expression of the reporter gene in tissue that contains replicating virus. If hosts containing constitutive promoter fusions to reporter genes are used, monitoring for hyper- or hypo-expression conditions of the reporter gene would be the focus. In this manner, genes that augment pathways that induce or upregulate the activity of certain promoters could be identified by following the surrogate marker of reporter gene expression. Conversely, gene that down-regulate or halt reporter gene expression could be identified as products that negatively effect the activities of the promoter or signaling pathway to which it is responsive. Virus vectors containing sequences that effected reporter gene expression by overexpression or suppression positive or negative regulatory factors can be isolated, and foreign gene contained may be sequenced and analyzed by bioinformatic methods.

### Example 36

Method to Induce the Expression of Alternative Splicing Variants to Discover Biological Effects in Host Organisms and to Use Said Host Organism as a Source for Novel cDNA Libraries Enriched for Alternatively Spliced Variants of Genes.

Transcription of nuclear genes in higher eukaryotic organisms results in a primary RNA transcript that contains both coding (exon) and non-coding (intron) information. A crucial step in RNA maturation before exporting to the cytosol for translation is the splicing of introns from the primary transcript and the rendering of contiguous exons for coding of the desired product. It is interesting to note that, although, splicing may occur in defined sites constitutively in certain gene, many genes can be spliced to produce multiple protein products, each with separate functions. The process of splicing out different sets of intron and splicing together of different array and order of exons for the same primary transcript is known is alternative splicing. This is powerful way genetic economy can be achieved in higher organisms to encode for multiple functions in a single gene cistron. The events of alternative splicing are regulated by families of small nuclear RNAs and associated proteins. These factors are responsible for the choice of splice sites used in primary RNA transcript and the nature of the mature mRNA reconstructed from the splicing process. Many alternative splicing events produce rare or tissue specific RNAs that result in the translation of specific protein products that have unique activities. The most famous of which is the alternative splicing of a Drosophila transcription factor results in the sex determination of the developing embryo. For a reference describing general alternative splicing, see Lopez, Ann. Rev. Genetics, 32 (1998), in press.

Since alternatively spliced mRNAs encode for proteins with differing functions, it would be interesting to investigate hosts that are deficient in these factors or hosts that no longer express such factors. It is difficult to accurately and effectively represent this diversity in standard cDNA libraries constructed from unaltered eukaryotic hosts. However, the use of virus expression vectors to overexpress or suppress the expression of factors involved in the splicing process will make it possible to increase the proportion of alternatively spliced mRNA in the host organism. Focused gene libraries will be constructed for the overexpression and the sense or antisense suppression of factors with potential and actual activities in the RNA splicing process in plants. Gene families can include the SF2/ASF-like group of splicing factors (Lopato et al.,PNAS 92:7672-7676 (1995)), the RS-rich family of splicing factors (Lapato et al., The Plant Cell 8:2255-2264 (1996)) and other splicing families that have been identified in the literature in lower or upper eukaryotic systems. The gene libraries will be sub-cloned into virus expression vectors and virus libraries will be inoculated as individuals or pools onto plants or plant cells. Once individual or groups of splicing factors are overexpressed or have their expression suppressed in plant cells, novel forms of splicing will occur due to the role of these proteins in alternative splicing of many transcription factors, splicing factors or other gene products. The high level of expression achieved by virus expression vectors and their ability to infect most cell types in plants should raise the overall level of aberrantly expressed mRNAs in the plant. The transfected plants will be used as the starting point for the isolation of poly A(+) RNA for the construction of cDNAs enriched for alternatively spliced genes. The alterations in the alternative splicing could be the splicing of a greater or lesser number of introns from the primary mRNA than normally occurs in non-transfected plants. These enriched cDNA libraries can now be cloned into virus expression vectors and the functions of these novel spliced forms of genes can be assayed on plants transfected with these vector libraries.

In this example, one can discover the plietropic functions of factors effecting alternative or normal splicing functions in plants from primary directed virus libraries with original splicing factor genes, or from virus libraries derived from plants containing induced novel spliced mRNAs.

Similar methods could be to derive novel cDNA libraries by using virus vectors to express factors responsible for transcriptional regulation of genes in plants. In this example, targeted cloning of transcription factor families would be ligated into virus expression vectors. Families could include homeodomain, Zn finger, leucine zipper and other transcription factor families appearing in pro or eukaryotic genomes. Schwechheimer, et al., Ann. Rev. Plant Phys. and Plant Mol. Biol. 49 (1998), in press. The gene libraries will be sub-cloned into virus expression vectors and virus libraries will be inoculated as individuals or pools onto plants or plant cells. Once individual or groups of transcription factors are overexpressed or have their expression suppressed in plant cells or plants, novel patterns of gene expression patterns will be induced. This will result in the appearance of a higher proportion of cDNAs normally present at low levels in the plant tissue or that are normally developmentally regulated. However, with the high level of expression achieved by virus expression vectors and their ability to infect most cell types in plants should induce these tissue specific cDNAs in aberrant cell types and at much higher than normal levels. The transfected plants will be used as the starting point for the isolation of poly A(+) RNA for the construction of cDNAs enriched for alternatively lowly expressed or developmentally expressed cDNAs. These cDNAs would be used to construct expression or gene suppression libraries that will be enriched for these rare or aberrantly expressed cDNAs. These enriched cDNA libraries can now be cloned into virus expression vectors and the functions of these novel spliced forms of genes can be assayed on plants transfected with these vector libraries.

Although the invention has been described with reference to the presently preferred embodiments, it should be understood that various modifications can be made without departing from the spirit of the invention. It is further understood that the instant invention applies to all plus stranded RNA viral vectors.

1. A method for identifying a gene function in a plant comprising a conditional lethal mutation in a gene comprising the steps of:
(a) growing one or more plants under first permissive conditions to produce a group of plants;
(b) growing a first set of the plants produced in step (a) under one or more restrictive conditions to determine the presence of a conditional lethal mutation;
(c) selecting one or more plants from step (b) that are sensitive to said restrictive conditions;
(d) growing a second set of the plants, which are produced in step (a) and genetically identical to those selected in step (c), under second permissive conditions to determine the growth requirement of plants having the conditional lethal mutation;
(e) growing a third set of the plants, which are produced in step (a) and genetically identical to those selected in step (c), under said restrictive conditions, and complementing a mutated gene of said selected plants by transfecting them with a viral vector containing an unmutated copy of a mutated gene, thereby identifying a gene function in a plant comprising a conditional lethal mutation in a gene.

2. A method for identifying a gene function in a plant which comprises a conditional lethal mutation in a gene, comprising:
(a) growing one or more plants under first permissive conditions;
(b) growing a set of plants produced in step (a) under one or more restrictive conditions;
(c) selecting one or more plants from step (b) that are sensitive to the restrictive condition;
(d) growing a set of plants selected in step (c) under a variety of permissive conditions;
(e) growing a set of plants selected in step (c) under a restrictive condition and complementing a mutated gene of the plants by transfecting the plants with a viral vector containing an unmutated copy of the mutated gene.

3. The method of claim 1 or 2, further comprising after step (e), the step of (f) isolating from said viral vector a gene complementing said mutation.

4. The method of claim 3, further comprising after the step of isolating said gene, a step selected from the group consisting of: (i) identifying the function of said gene, (ii) identifying the product expressed by said gene, and (iii) sequencing said gene.

5. The method of claim 1 or 2, wherein the first permissive conditions include a complete growth medium for the plant tissue, plant cell or plant organ.

6. The method of claim 1 or 2, wherein the first permissive conditions include a growth medium at low osmotic strength.

7. The method of claim 1 or 2, wherein the first permissive conditions include a temperature between about 5 and 15° C. below the optimal growth temperature for a wild type uninfected plant.

8. The method of claim 1 or 2, wherein the restrictive conditions include a temperature between the optimal growth temperature for the organism and at least about 15° C. above the optimal growth temperature for the organism.

9. The method of claim 1 or 2, wherein the second permissive conditions are substantially the same as the first permissive conditions.

10. The method of claim 1 or 2, wherein the plant cells in growing step (a) are replica plated plant cells on plant leaf disks.

11. The method of claim 1 or 2, wherein the period of time in step (c) is equivalent to at least one growth cycle.

12. The method of claim 1 or 2, wherein the plants from step (a) are selected from the group consisting of monocotyledons and dicotyledons.

13. The method of claim 1 or 2, wherein the plants from step (a) have been mutagenized by insertion mutagenesis with T-DNA or transposon nucleic acid sequences.

14. The method of claim 13, wherein the plants have been mutagenized with a mutagen selected from the group consisting of nucleic acid alkylating agents, intercalating agents, ionizing radiation, heat, and sound.

15. The method of claim 14, wherein said alkylating and intercalating agents are selected from the group consisting of methanesulfonate, methyl methanesulfonate, methylnitrosoguanidine, 4-nitroquinoline-1-oxide, 2-aminopurine, 5-bromouracil, ICR 191 and other acridine derivatives, ethidium bromide, nitrous acid, and N-methyl-N′-nitroso-N-nitroguanidine.

16. The method according to claim 1, wherein said plant is a transgenic plant.

17. The method according to claim 1, wherein said plant is Nicotiana benthamiana, Nicotiana tabacum or Arabidopsis thaliana.

18. The method according to claim 1, wherein said viral vector is derived from a single-stranded plus sense plant RNA virus.

19. The method according to claim 18, wherein said viral vector is derived from a tobacco mosaic virus, tomato mosaic virus, or ribgrass mosaic virus.

20. A method for identifying a gene function in a plant carrying a conditional lethal mutation in a gene, comprising:
(a) crossing to itself, a plant that is heterozygous for a conditional lethal mutation to make a homozygous mutant plant; and
(a) growing the plant from step (a) under a restrictive condition and complementing a mutated gene of the plant by transfecting it with a viral vector containing an unmutated copy of the mutated gene.


SEQUENCE LISTING (49 sequences)

SEQ ID NO: 1 - DNA, 26 bp, viral
ctcgcaaagtttcgaaccaaatcctc

SEQ ID NO: 2 - DNA, 35 bp, VIRAL
cggggtacctgggccccaaccgggggttccggggg

SEQ ID NO: 3 - DNA, 41 bp, VIRAL
tcctcgagcctaggctcgcaaagtttcgaaccaaatcctca

SEQ ID NO: 4 - DNA, 35 bp, VIRAL; identical to SEQ ID NO: 2

SEQ ID NO: 5 - DNA, 24 bp, VIRAL
tatgtatggtgcagaagaacagat

SEQ ID NO: 6 - DNA, 25 bp, VIRAL
agtcgactcttcctcttctggcatc

SEQ ID NO: 7 - DNA, 30 bp, VIRAL
tgctcgagtgtgttcttcagttttctgtca

SEQ ID NO: 8 - DNA, 30 bp, VIRAL
aactcgagcgctttgatttctccgaagctt

SEQ ID NO: 9 - DNA, 39 bp, VIRAL
gcctcgagtgcagcatggaaacccttctaaagcttttcc

SEQ ID NO: 10 - DNA, 36 bp, VIRAL
tccctaggtcaaaggctctctattgctagattgccc

SEQ ID NO: 11 - DNA, 44 bp, VIRAL
ccaagcttctcgagtgcagcatgcagcaaccgccgctgcttgac

SEQ ID NO: 12 - DNA, 43 bp, VIRAL
aagatctctcgagctaaacgggacgctgccaaagaccggccgg

SEQ ID NO: 13 - DNA, 26 bp, VIRAL
ggctgtgaaactcgaaaaggttccgg

SEQ ID NO: 14 - DNA, 36 bp, VIRAL
cggggtacctgggccgctaccggcggttaggggagg

SEQ ID NO: 15 - DNA, 32 bp, VIRAL
gacagaagctttgcagcatgcaaaaaaccgtt

SEQ ID NO: 16 - DNA, 31 bp, VIRAL
cgcggtcattgcagatcctcaatcatcaggc

SEQ ID NO: 17 - DNA, 25 bp, VIRAL
ttgcactctgttggctcatgacgat

SEQ ID NO: 18 - DNA, 26 bp, VIRAL
caagcttgcacaaacgaacgtctcac

SEQ ID NO: 19 - DNA, 42 bp, VIRAL
cactcgagagcatggctattcccgaagaatttgatattatcg

SEQ ID NO: 20 - DNA, 36 bp, VIRAL
tccctaggttagaatctagcaagaccggtcttctcg

SEQ ID NO: 21 - DNA, 38 bp, VIRAL
ctctcgagatcaatcatccatctccgaagtgtgtctgc

SEQ ID NO: 22 - DNA, 34 bp, VIRAL
tccctaggtcagattttctcccagattgcgtagc

SEQ ID NO: 23 - DNA, 42 bp, VIRAL
gcctcgagtgcagcatgatcagattcttagtcctctctttgc

SEQ ID NO: 24 - DNA, 36 bp, VIRAL
tccctaggctaaatagcataacttccacatcaaagc

SEQ ID NO: 25 - DNA, 38 bp, VIRAL
cactcgagagcatgctgcacctgactcctgaggagaag

SEQ ID NO: 26 - DNA, 38 bp, VIRAL
cgtctagattagtgatacttgtgggccagcgcattagc

SEQ ID NO: 27 - DNA, 26 bp, VIRAL
ggctgtgaaactcgaaaaggttccgg

SEQ ID NO: 28 - DNA, 36 bp, VIRAL; identical to SEQ ID NO: 14

SEQ ID NO: 29 - DNA, 31 bp, VIRAL
tactcgaggttcataagaccgcggtaggcgg

SEQ ID NO: 30 - DNA, 36 bp, VIRAL
cggggtacctgggcccctacccggggtttagggagg

SEQ ID NO: 31 - DNA, 1536 bp, VIRAL
ctcgaggttcataagaccgcggtaggcggagcgtttgtttactgtagtataattaaatatttgtcagataaaaggttgtttaaagatttgttttttgtttgactcgataatgtcttacgagcctaaagttagtgacttccttgctcttacgaaaaaggaggaaattttacccaaggctttgacgagattaaagactgtctctattagtactaaggatgttatatctgttaaggagtctgagtccctgtgtgatattgatttgttagtgaatgtgccattagataagtataggtatgtgggtgttttgggtgttgttttcaccggtgaatggctggtaccggattgttaaaggtggggtaacagtgagcgtgattgacaaacggcttgaaaattccagagagtgcataattggtacgtaccgagctgctgtaaaggacagaaggttccagttcaagctggttccaaattacttcgtatccattgcggatgccaagcgaaaaccgtggcaggttcatgtgcgaatcaaaatctgaagatcgaagctggatggcaacctctagctctagaggtggtttctgttgccatggttactaataacgtggttgttaaaggtttgagggaaaaggtcatcgcagtgaatgtccgaacgtcgaaggtttcgaaggtgtggttgacgatttcgtcgattcggttgctgcattcaaggcgattgacagtttccgaaagaaaaaagaaaaagattggaggaagggatgtaaataataataagtatagatatagaccggagagatacgccggtcctgattcgttacaatataaagaaaaaaatggtttacaacatcacgagctcgaatcagtaccagtatttcgcagcgatgtgggcagagcccacagcgatgcttaaccagtgcgtgtctgcgttgtcgcaatcgtatcaaactcaggcggcaagagatactgttagacagcagttctctaaccttctgagtgcgattgtgacaccgaaccagcggtttccagaaacaggataccgggtgtatattaattcagcagttctaaaaccgttgtacgagtctctcatgaagtcctttgatactagaaataggatcattgaaactgaagaagagtcgcgtccatcggcttccgaagtatctaatgcaacacaacgtgttgatgatgcgaccgtcggccatcaggagtcaaattcagcttttgctgaacgagctctccaacggacatggtctgatcgaacagggcagagttcgaggttttattaccttgggctactgcgccagctacataggcgtggtgcacacgatagtgcatagtgtttttctctccacttaaatcgaagagatatacttacggtgtaattccgcaagggtggcgtaaaccaaattacgcaatgttttaggttccatttaaatcgaaacctgttatttcctggatcacctgttaacgtacgcgtggcgtatattacagtgggaataactaaaagtgagagttcgaatcctcctaaccccgggtaggggccca

SEQ ID NO: 32 - DNA, 55 bp, VIRAL
gatggcgccttaatacgacgcactatagttttatttttgttgcaacaacaacaac

SEQ ID NO: 33 - DNA, 30 bp, VIRAL
cttgtgcccttcatgacgagctatatcacg

SEQ ID NO: 34 - DNA, 498 bp, VIRAL
ccttaatacgactcactatagttttatttttgttgcaacaacaacaacaaattacaataacaacaaaacaaatacaaacaacaacaacatggcacaatttcaacaaacagtaaacatgcaaacattccaggctgccgcagggcgcaacagcctggtgaatgatttagcctcacgacgtgtttatgacaatgctgtcgaggagctaaatgcacgctcgagacgccctaaggttcattactccaaatcagtgtctacggaacagacgctgttagcttcaaacgcttatccggagtttgacgatttcctttactcatacccaaacatgccgtacactcccttgcgggtggcctaaggactcttgagttagagtatctcatgatgcaagttccgttcggttctctgacgtacgacatcggtggtaactttgcagcgcaccttttcaaaggacgcgactacgttcactgctgtatgccaaacttggatgtacgtgatatagct

SEQ ID NO: 35 - DNA, 55 bp, VIRAL
gatggcgccttaatacgactcactatagttttatttttgttgcaacaacaacaac

SEQ ID NO: 36 - DNA, 37 bp, VIRAL
atcgtttaaactgggcccctacccggggttagggagg

SEQ ID NO: 37 - DNA, 496 bp, VIRAL
ccttaatacgactcactatagttttatttttgttgcaacaacaacaacaaattacaataacaacaaaacaaatacaaacaacaacaacatggcacaatttcaacaaacagtaaacatgcaaacattccaggctgccgcaggcgcaacagcctggtgaatgatttagcctcacgacgtgtttatgacaatgctgtcgaggagctaaatgcacgctcgagacgccctaaggttcattactccaaatcagtgtctacggaacagacgctgttagcttcaaacgcttatccggagtttgagatttcctttactcatacccaaacatgccgtacactcccttgcgggtggcctaaggactcttgagttagagtatctcatgatgcaagttccgttcggttctctgacgtacgacatcggtggtaactttgcagcgcaccttttcaaaggacgcgactacgttcactgctgtatgccaaacttggatgtacgtgatatagct

SEQ ID NO: 38 - DNA, 55 bp, VIRAL; identical to SEQ ID NO: 35

SEQ ID NO: 39 - DNA, 37 bp, VIRAL; identical to SEQ ID NO: 36

SEQ ID NO: 40 - DNA, 14 bp, VIRAL
tcgagcggccgcat

SEQ ID NO: 41 - DNA, 8 bp, VIRAL
gcggccgc

SEQ ID NO: 42 - protein, 19 aa, VIRAL
SYVPSAEQILEFVKQISSQ

SEQ ID NO: 43 - DNA, 773 bp, VIRAL
  CDS (1)...(1992)
ccgaaacattcttcgtagtgaagcaaaatggggttgagtttcgccaagctgtttagcaggctttttgccaagaaggagatgcgaattctgatggttggtcttgatgctgctggtaagaccacaatcttgtacaagctcaagctcggagagattgtcaccaccatccctactattggtttcaatgtggaaactgtggaatacaagaacattagtttcaccgtgtgggatgtcgggggtcaggacaagatccgtcccttgtgagacactacttccagaacactcaaggtctaatctttgttgttgatagcaatgacagagacagagttgttgaggctcgagatgaactccacaggatgctgaatgaggacgagctgcgtgatgctgtgttgcttgtgtttgccaacaagcaagatcttccaaatgctatgaacgctgctgaaatcacagataagcttggccttcactccctccgtcagcgtcattggtatatccagagcacatgtgccacttcaggtgaagggctttatgaaggtctggactggctctccaacaacatcgctggcaaggcatgatgagggagaaattgcgttgcatcgagatgattctgtctgctgtgttgggatctctctctgtcttgatgcaagagagattataaatattatctgaacctttttgcttttttgggtatgtgaatgtttcttattgtgcaagtagatggtcttgtacctaaaaatttactagaagaacccttttaaatagctttcgtgtattgt

SEQ ID NO: 44 - DNA, 41 bp, viral
gcctcgagtgcagcatggggttgtcattcggaaagttgttc

SEQ ID NO: 45 - DNA, 33 bp, viral
tacctaggccttgcttgcgatgttgttggagag

SEQ ID NO: 46 - DNA, 749 bp, viral
tttcgatctaaggttcgtgatctccttcttctctacgaagtttacactttttcttcaaaggaaacaatgagccagtacaatcaacctcccgttggtgttcctcctcctcaaggttatccaccggagggatatccaaaagatgcttatccaccacaaggatatcctcctcagggatatcctcagcaaggctatccacctcagggatatcctcaacaaggttatcctcagcaaggatatcctccaccgtacgcgcctcaatatcctccaccaccgcaagcatcagcaacaacagagcaagtcctggctttctagaaggatgtcttgctgctctgtgttgttgctgtctcttggatgcttgcttctgattggagtctctctctctctgcataaagcttcgggatttatttgtaagagggtttttgggttaaacaaaaaccttaattgatttgtggggcattaaaaatgaatctctcgatgattctcttcgttatgtggtaatgttcttcggttataacatttaacattgctatcgacgttctgcctagttggatttgattattgggaatgtaaattggttgggaagacaccgggccgttaatgacagaacccgaactgagatggagtatgatctgaaatatttaaaacaatcctcgcgacatagcctccaatctcatcgtaaatattctttttaaactattcccaatcttaacttttatagtctggtcgactgaccactactctttttcctt

SEQ ID NO: 47 - DNA, 350 bp, Viral
cttcactttcgccgatggctctacctaaccagcaaaccgtggattaccctagcttcaagctcgttatcgttggcgatggaggcacagggaagaccacatttgtaaagagacatcttactggagagtttgagaagaagtatgaacccactattggtgttgaggttcatcctcttgatttcttcactaactgtggcaagatccgtttctactgttgggatactgctggccaagagaaatttggtggtcttagggatggttactacatccatggacaatgtgctatcatcatgtttgatgtcacaagcacgactgacatacaagaatgttccaacatggcaccgtgatctttg

SEQ ID NO: 48 - DNA, 29 bp, Viral
ggccctaggatggctctagttgttaaagg

SEQ ID NO: 49 - DNA, 34 bp, VIRAL
gttcttctcctttgctagccatttaattaatgac